(19) United States
(12) Patent Application Publication
(10) Pub. No.: US 2003/0077568 A1
(43) Pub. Date: Apr. 24, 2003

(54) Methods of diagnosis of colorectal cancer, compositions and methods of screening for colorectal cancer modulators

(76) Inventors: Kurt C. Gish, Redwood City, CA (US); David H. Mack, San Francisco, CA (US); Keith E. Wilson, Menlo Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/930,020

(22) Filed: Aug. 14, 2001

Related U.S. Application Data
(63) Continuation-in-part of application No. 09/663,733, filed on Sep. 15, 2000, now abandoned.

(51) Int. Cl. C12Q 1/00; C12Q 1/68; G01N 33/574

(52) U.S. Cl. 435/4; 435/6; 435/7.23

(57) ABSTRACT

Described herein are methods that can be used for diagnosis and prognosis of colorectal cancer. Also described herein are methods that can be used to screen candidate bioactive agents for the ability to modulate colorectal cancer. Additionally, methods and molecular targets (genes and their products) for therapeutic intervention in colorectal and other cancers are described.

### CROSS-REFERENCES TO RELATED APPLICATIONS

This application is a continuation in part of U.S. patent application Ser. No. 09/663,733 filed Sep. 15, 2000, which is incorporated herein by reference in its entirety.

### FIELD OF THE INVENTION

The invention relates to the identification of expression profiles and the nucleic acids involved in colorectal cancer, and to the use of such expression profiles and nucleic acids in diagnosis and prognosis of colorectal cancer. The invention further relates to methods for identifying and using candidate agents and/or targets which modulate colorectal cancer.

### BACKGROUND OF THE INVENTION

Cancer of the colon and/or rectum (referred to as “colorectal cancer”) are significant in Western populations and particularly in the United States. Cancers of the colon and rectum occur in both men and women most commonly after the age of 50. These develop as the result of a pathologic transformation of normal colon epithelium to an invasive cancer. There have been a number of recently characterized genetic alterations that have been implicated in colorectal cancer, including mutations in two classes of genes, tumor-suppressor genes and proto-oncogenes, with recent work suggesting that mutations in DNA repair genes may also be involved in tumorigenesis. For example, inactivating mutations of both alleles of the adenomatous polyposis coli (APC) gene, a tumor suppressor gene, appears to be one of the earliest events in colorectal cancer, and may even be the initiating event. Other genes implicated in colorectal cancer include the MCC gene, the p53 gene, the DCC (deleted in colorectal carcinoma) gene and other chromosome 18q genes, and genes in the TGF-β signaling pathway. For a review, see Molecular Biology of Colorectal Cancer, pp. 238-299, in Curr. Probl. Cancer, September/October 1997; see also Willams, Colorectal Cancer (1996); Kinsella & Schofield, Colorectal Cancer: A Scientific Perspective (1993); Colorectal Cancer: Molecular Mechanisms, Premalignant State and its Prevention (Schmiegel & Scholmerich eds., 2000); Colorectal Cancer: New Aspects of Molecular Biology and Their Clinical Applications (Hanski et al., eds 2000); McArdle et al., Colorectal Cancer (2000); Wanebo, Colorectal Cancer (1993); Levin, The American Cancer Society: Colorectal Cancer (1999); Treatment of Hepatic Metastases of Colorectal Cancer (Nordlinger & Jaeck eds., 1993); Management of Colorectal Cancer (Dunitz et al., eds. 1998); Cancer: Principles and Practice of Oncology (Devita et al., eds. 2001); Surgical Oncology: Contemporary Principles and Practice (Kirby et al., eds. 2001); Offit, Clinical Cancer Genetics: Risk Counseling and Management (1997); Radioimmunotherapy of Cancer (Abrams & Fritzberg eds. 2000); Fleming, AJCC Cancer Staging Handbook (1998); Textbook of Radiation Oncology (Leibel & Phillips eds. 2000); and Clinical Oncology (Abeloff et al., eds. 2000).

Imaging of colorectal cancer for diagnosis has been problematic and Ad limited. In addition, metastasis of the tumor to the lumen, and metastasis of tumor cells to at regional lymph nodes are important prognostic factors (see, e.g., PET in Oncology: Basics and Clinical Application (Ruhlmann et al. eds. 1999). For example, five year survival rates drop from 80 percent in patients with no lymph node metastases to 45 to 50 percent in those patients who do have lymph node metastases. A recent report showed that micrometastases can be detected from lymph nodes using reverse transcriptase-PCR methods based on the presence of mRNA for carcinoembryonic antigen, which has previously been shown to be present in the vast majority of colorectal cancers but not in normal tissues. Liefers et al., New England J. of Med. 339(4):223 (1998).

Thus, methods that can be used for diagnosis and prognosis of colorectal cancer would be desirable. Accordingly, provided herein are methods that can be used in diagnosis and prognosis of colorectal cancer. Further provided are methods that can be used to screen candidate bioactive agents for the ability to modulate colorectal cancer. Additionally, provided herein are molecular targets for therapeutic intervention in colorectal and other cancers.

### BRIEF SUMMARY OF THE INVENTION

The present invention provides novel methods for diagnosis and prognosis evaluation for colorectal cancer, as well as methods for screening for compositions which modulate colorectal cancer. Methods of treatment of colorectal cancer, as well as compositions, are also provided herein.

In one aspect, a method of screening drug candidates comprises providing a cell that expresses an expression profile gene selected from those of Table I. The method further includes adding a drug candidate to the cell and determining the effect of the drug candidate on the expression of the expression profile gene.

In one embodiment, the method of screening drug candidates includes comparing the level of expression in the absence of the drug candidate to the level of expression in the presence of the drug candidate, wherein the concentration of the drug candidate can vary when present, and wherein the comparison can occur after addition or removal of the drug candidate. In a preferred embodiment, the cell expresses at least two expression profile genes. The profile genes may show an increase or decrease.

Also provided herein is a method of screening for a bioactive agent capable of binding to a colorectal cancer modulator protein, the method comprising combining the colorectal cancer modulator protein and a candidate bioactive agent, and determining the binding of the candidate agent to the colorectal cancer modulator protein. Preferably the colorectal cancer modulator protein is a product encoded by a gene of Table 1 or Table 2 .

Further provided herein is a method for screening for a bioactive agent capable of modulating the activity of a colorectal cancer modulator protein. In one embodiment, the method comprises combining the colorectal cancer modulator protein and a candidate bioactive agent, and determining the effect of the candidate agent on the bioactivity of the colorectal cancer modulator protein. Preferably the colorectal cancer modulator protein is a product encoded by a gene of Table 1 or Table 2.

Also provided is a method of evaluating the effect of a candidate colorectal cancer drug comprising administering the drug to a transgenic animal expressing or over-expressing the colorectal cancer modulator protein, or an animal lacking the colorectal cancer modulator protein, for example as a result of a gene knockout.

Additionally, provided herein is a method of evaluating the effect of a candidate colorectal cancer drug comprising administering the drug to a patient and removing a cell sample from the patient. The expression profile of the cell is then determined. This method may further comprise comparing the expression profile to an expression profile of a healthy individual. In a preferred embodiment, said expression profile includes a gene of Table 1 or Table 2.

Moreover, provided herein is a biochip comprising one or more nucleic acid segments of Table 1 or Table 2, wherein the biochip comprises fewer than 1000 nucleic acid probes. Preferable at least two nucleic acid segments are included.

Furthermore, a method of diagnosing a disorder associated with colorectal cancer is provided. The method comprises determining the expression of a gene of Table 1 or Table 2, in a first tissue type of a first individual, and comparing the distribution to the expression of the gene from a second normal tissue type from the first individual or a second unaffected individual. A difference in the expression indicates that the first individual has a disorder associated with colorectal cancer.

In another aspect, the present invention provides an antibody which specifically binds to a protein encoded by a nucleic acid of Table 1 or Table 2 or a fragment thereof. Preferably the antibody is a monoclonal antibody. The antibody can be a fragment of an antibody such as a single stranded antibody as further described herein, or can be conjugated to another molecule. In one embodiment, the antibody is a humanized antibody.

In one embodiment a method for screening for a bioactive agent capable of interfering with the binding of a colorectal cancer modulating protein (colorectal cancer modulator protein) or a fragment thereof and an antibody which binds to said colorectal cancer modulator protein or fragment thereof. In a preferred embodiment, the method comprises combining a colorectal cancer modulator protein or fragment thereof, a candidate bioactive agent and an antibody which binds to said colorectal cancer modulator protein or fragment thereof. The method further includes determining the binding of said colorectal cancer modulator protein or fragment thereof and said antibody. Wherein there is a change in binding, an agent is identified as an interfering agent. The interfering agent can be an agonist or an antagonist. Preferably, the agent inhibits colorectal cancer.

In a further aspect, a method for inhibiting colorectal cancer is provided. The method can be performed in vitro or in vivo, preferably in vivo to an individual. In a preferred embodiment the method of inhibiting colorectal cancer is provided to an individual with cancer. As described herein, methods of inhibiting colorectal cancer can be performed by administering an inhibitor of the activity of a protein encoded by a nucleic acid of Table 1 or Table 2, including an antisense molecule to the gene or its gene product.

Also provided herein are methods of eliciting an immune response in an individual. In one embodiment a method provided herein comprises administering to an individual a composition comprising a colorectal cancer modulating protein, or a fragment thereof. In another embodiment, the protein is encoded by a nucleic acid selected from those of Table 1 or Table 2. In another aspect, said composition comprises a nucleic acid comprising a sequence encoding a colorectal cancer modulating protein, or a fragment thereof.

Further provided herein are compositions capable of eliciting an immune response in an individual. In one embodiment, a composition provided herein comprises a colorectal cancer modulating protein, preferably encoded by a nucleic acid of Table 1 or Table 2, or a fragment thereof, and a pharmaceutically acceptable carrier. In another embodiment, said composition comprises a nucleic acid comprising a sequence encoding a colorectal cancer modulating protein, preferably selected from the nucleic acids of Table 1 or Table 2 and a pharmaceutically acceptable carrier.

Also provided are methods of neutralizing the effect of a colorectal cancer protein, or a fragment thereof, comprising contacting an agent specific for said protein with said protein in an amount sufficient to effect neutralization. In another embodiment, the protein is encoded by a nucleic acid selected from those of Table 1 or Table 2.

In another aspect of the invention, a method of treating an individual for colorectal cancer is provided. In one embodiment, the method comprises administering to said individual an inhibitor of a colorectal cancer modulating protein. In another embodiment, the method comprises administering to a patient having colorectal cancer an antibody to a colorectal cancer modulating protein conjugated to a therapeutic moiety. Such a therapeutic moiety can be a cytotoxic agent or a radioisotope.

Compounds and compositions are also provided. Other aspects of the invention will become apparent to the skilled artisan by the following description of the invention.

### BRIEF DESCRIPTION OF THE DRAWINGS

[NOT APPLICABLE]

### DETAILED DESCRIPTION OF THE INVENTION

The present invention provides novel methods for diagnosis and prognosis evaluation for colorectal cancer, as well as methods for screening for compositions which modulate colorectal cancer. The methods herein are related to those of U.S. patent application Ser. No. 09/525,993 and International Patent Application No. PCT/US00/07044, each of which is incorporated herein in its entirety.

By “colorectal cancer” herein is meant a colon and/or rectal tumor or cancer that is classified as Dukes stage A or B as well as metastatic tumors classified as Dukes stage Cor D (see, e.g., Cohen et al., Cancer of the Colon, in Cancer: Principles and Practice of Oncology, pp. 1144-1197 (Devita et al., eds., 5^{ed}. 1997); see also Harrison's Principles of Internal Medicine, pp. 1289-129 (Wilson et al., eds., ₁₂^{th} ed., 1991). “Treatment, monitoring, detection or modulation of colorectal cancer” includes treatment, monitoring, detection, or modulation of colorectal disease in those patients who have colorectal disease (Dukes stage A , B, C or D) in which gene expression from a gene in Table 1 or 2, is increased or decreased, indicating that the subject is more likely to progress to metastatic disease than a patient who does not have an increase or decrease in gene expression of a gene in Table 1 or 2. In Dukes stage A, the tumor has penetrated into, but not through, the bowel wall. In Dukes stage B, the tumor has penetrated through the bowel wall but there is not yet any lymph involvement. In Dukes stage C, the cancer involves regional lymph nodes. In Dukes stage D, there is distant metastasis, e.g., liver, lung, etc.

Table 1 provides unigene cluster identification numbers for the nucleotide sequence of genes that exhibit increased expression in colorectal cancer samples. Tables 1 also provides an exemplar accession number that provides a nucleotide sequence that is part of the unigene cluster. Table 2 provides the nucleic acid and protein sequence of the CBF9 gene as well as the Unigene and Exemplar accession numbers for CBF9.

In one aspect, the expression levels of genes are determined in different patient samples for which either diagnosis or prognosis information is desired, to provide expression profiles. An expression profile of a particular sample is essentially a “fingerprint” of the state of the sample; while two states may have any particular gene similarly expressed, the evaluation of a number of genes simultaneously allows the generation of a gene expression profile that is unique to the state of the cell. That is, normal tissue may be distinguished from colorectal cancer tissue, and within colorectal cancer tissue, different prognosis states (good or poor long term survival prospects, for example) may be determined. By comparing expression profiles of colon tissue in known different states, information regarding which genes are important (including both up- and down-regulation of genes) in each of these states is obtained. The identification of sequences that are differentially expressed in colorectal cancer versus normal colon tissue, as well as differential expression resulting in different prognostic outcomes, allows the use of this information in a number of ways. For example, the evaluation of a particular treatment regime may be evaluated: does a chemotherapeutic drug act to improve the long-term prognosis in a particular patient. Similarly, diagnosis may be done or confirmed by comparing patient samples with the known expression profiles. Furthermore, these gene expression profiles (or individual genes) allow screening of drug candidates with an eye to mimicking or altering a particular expression profile; for example, screening can be done for drugs that suppress the colorectal cancer expression profile or convert a poor prognosis profile to a better prognosis profile. This may be done by making biochips comprising sets of the important colorectal cancer genes, which can then be used in these screens. These methods can also be done on the protein basis; that is, protein expression levels of the colorectal cancer proteins can be evaluated for diagnostic and prognostic purposes or to screen candidate agents. In addition, the colorectal cancer nucleic acid sequences can be administered for gene therapy purposes, including the administration of antisense nucleic acids, or the colorectal cancer proteins (including antibodies and other modulators thereof) administered as therapeutic drugs.

Thus the present invention provides nucleic acid and protein sequences that are differentially expressed in colorectal cancer, herein termed “colorectal cancer sequences”. As outlined below, colorectal cancer sequences include those that are up-regulated (i.e. expressed at a higher level) in colorectal cancer, as well as those that are down-regulated (i.e. expressed at a lower level) in colorectal cancer . In a preferred embodiment, the colorectal cancer sequences are from humans; however, as will be appreciated by those in the art, colorectal cancer sequences from other organisms may be useful in animal models of disease and drug evaluation; thus, other colorectal cancer sequences are provided, from vertebrates, including mammals, including rodents (rats, mice, hamsters, guinea pigs, etc.), primates, farm animals (including sheep, goats, pigs, cows, horses, etc.). colorectal cancer sequences from other organisms may be obtained using the techniques outlined below.

Colorectal cancer sequences can include both nucleic acid and amino acid sequences. In a preferred embodiment, the colorectal cancer sequences are recombinant nucleic acids. By the term “recombinant nucleic acid” herein is meant nucleic acid, originally formed in vitro, in general, by the manipulation of nucleic acid by polymerases and endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a colorectal cancer protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

In a preferred embodiment, the colorectal cancer sequences are nucleic acids. As will be appreciated by those in the art and is more fully outlined below, colorectal cancer sequences are useful in a variety of applications, including diagnostic applications, which will detect naturally occurring nucleic acids, as well as screening applications; for example, biochips comprising nucleic acid probes to the colorectal cancer sequences can be generated. In the broadest sense, then, by “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done for a variety of reasons, for example to increase the stability and half-life of such molecules in physiological environments or as probes on a biochip.

As will be appreciated by those in the art, all of these nucleic acid analogs may find use in the present invention. In addition, mixtures of naturally occurring nucleic acids and analogs can be made; alternatively, mixtures of different nucleic acid analogs, and mixtures of naturally occurring nucleic acids and analogs may be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tm for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

The nucleic acids may be single stranded or double stranded, as specified, or contain portions of both double stranded or single stranded sequence. As will be appreciated by those in the art, the depiction of a single strand (“Watson”) also defines the sequence of the other strand (“Crick”); thus the sequences described herein also includes the complement of the sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

A colorectal cancer sequence can be initially identified by substantial nucleic acid and/or amino acid sequence homology to the colorectal cancer sequences outlined herein. Such homology can be based upon the overall nucleic acid or amino acid sequence, and is generally determined as outlined below, using either homology programs or hybridization conditions.

The isolation of mRNA comprises isolating total cellular RNA by disrupting a cell and performing differential centrifugation. Once the total RNA is isolated, mRNA is isolated by making use of the adenine nucleotide residues known to those skilled in the art as a poly (A) tail found on virtually every eukaryotic mRNA molecule at the 3′ end thereof. Oligonucleotides composed of only deoxythymidine [olgo(dT)] are linked to cellulose and the oligo(dT)-cellulose packed into small columns. When a preparation of total cellular RNA is passed through such a column, the mRNA molecules bind to the oligo(dT) by the poly (A) tails while the rest of the RNA flows through the column. The bound mRNAs are then eluted from the column and collected.

The colorectal cancer sequences of the invention can be identified as follows. Samples of normal and tumor tissue are applied to biochips comprising nucleic acid probes. The samples are first microdissected, if applicable, and treated as described above for the preparation of mRNA. Suitable biochips are commercially available, for example from Affymetrix. Gene expression profiles as described herein are generated, and the data analyzed.

In a preferred embodiment, the genes showing changes in expression as between normal and disease states are compared to genes expressed in other normal tissues, including, but not limited to lung, heart, brain, liver, breast, kidney, muscle, prostate, small intestine, large intestine, spleen, bone, and placenta. In a preferred embodiment, those genes identified during the colorectal cancer screen that are expressed in any significant amount in other tissues are removed from the profile, although in some embodiments, this is not necessary. That is, when screening for drugs, it is preferable that the target be disease specific, to minimize possible side effects.

In a preferred embodiment, colorectal cancer sequences are those that are up-regulated in colorectal cancer ; that is, the expression of these genes is higher in colorectal carcinoma as compared to normal colon tissue. “Up-regulation” as used herein means at least about a 1.1 fold change, preferably a 1.5 or two fold change, preferably at least about a three fold change, with at least about five-fold or higher being preferred. All accession numbers herein are for the GenBank sequence database and the sequences of the accession numbers are hereby expressly incorporated by reference. GenBank is known in the art, see, e.g., Benson, D A, et al., Nucleic Acids Research 26:1-7 (1998) and http://www.ncbi.nlm.nih.gov/. In addition, these genes were found to be expressed in a limited amount or not at all in heart, brain, lung, liver, breast, kidney, prostate, small intestine and spleen.

In a preferred embodiment, colorectal cancer sequences are those that are down-regulated in colorectal cancer ; that is, the expression of these genes is lower in colorectal carcinoma as compared to normal colon tissue. “Down-regulation” as used herein means at least about a two-fold change, preferably at least about a three fold change, with at least about five-fold or higher being preferred.

Colorectal cancer proteins of the present invention may be classified as secreted proteins, transmembrane proteins or intracellular proteins. In a preferred embodiment the colorectal cancer protein is an intracellular protein. Intracellular proteins may be found in the cytoplasm and/or in the nucleus. Intracellular proteins are involved in all aspects of cellular function and replication (including, for example, signaling pathways); aberrant expression of such proteins results in unregulated or disregulated cellular processes. For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing intracellular proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate.

In a preferred embodiment, the colorectal cancer sequences are transmembrane proteins. Transmembrane proteins are molecules that span the phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors are classified as “seven transmembrane domain” proteins, as they contain 7 membrane spanning regions. Important transmembrane protein receptors include, but are not limited to insulin receptor, insulin-like growth factor receptor, human growth hormone receptor, glucose transporters, transferrin receptor, epidermal growth factor receptor, low density lipoprotein receptor, epidermal growth factor receptor, leptin receptor, interleukin receptors, e.g. IL-1 receptor, IL-2 receptor, etc.

Characteristics of transmembrane domains include approximately 20 consecutive hydrophobic amino acids that may be followed by charged amino acids. Therefore, upon analysis of the amino acid sequence of a particular protein, the localization and number of transmembrane domains within the protein may be predicted.

The extracellular domains of transmembrane proteins are diverse; however, conserved motifs are found repeatedly among various extracellular domains. Conserved structure and/or functions have been ascribed to different extracellular motifs. For example, cytokine receptors are characterized by a cluster of cysteines and a WSXWS (W=tryptophan, S=serine, X=any amino acid) motif. Immunoglobulin-like domains are highly conserved. Mucin-like domains may be involved in cell adhesion and leucine-rich repeats participate in protein-protein interactions.

Many extracellular domains are involved in binding to other molecules. In one aspect, extracellular domains are receptors. Factors that bind the receptor domain include circulating ligands, which may be peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, neurotrophic factors and the like. Extracellular domains also bind to cell-associated molecules. In this respect, they mediate cell-cell interactions. Cell-associated ligands can be tethered to the cell for example via a glycosylphosphatidylinositol (GPI) anchor, or may themselves be transmembrane proteins. Extracellular domains also associate with the extracellular matrix and contribute to the maintenance of the cell structure.

Colorectal cancer proteins that are transmembrane are particularly preferred in the present invention as they are good targets for immunotherapeutics, as are described herein. In addition, as outlined below, transmembrane proteins can be also useful in imaging modalities.

It will also be appreciated by those in the art that a transmembrane protein can be made soluble by removing transmembrane sequences, for example through recombinant methods. Furthermore, transmembrane proteins that have been made soluble can be made to be secreted through recombinant means by adding an appropriate signal sequence.

In a preferred embodiment, the colorectal cancer proteins are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. colorectal cancer proteins that are secreted proteins are particularly preferred in the present invention as they serve as good targets for diagnostic markers, for example for blood tests.

A colorectal cancer sequence is initially identified by substantial nucleic acid and/or amino acid sequence homology to the colorectal cancer sequences outlined herein. Such homology can be based upon the overall nucleic acid or amino acid sequence, and is generally determined as outlined below, using either homology programs or hybridization conditions.

As used herein, the terms “colorectal cancer nucleic acid”, “colorectal cancer protein” or “colorectal cancer polynucleotide” or “colorectal cancer-associated transcript” refers to nucleic acid and polypeptide polymorphic variants, alleles, mutants, and interspecies homologs that: (1) have a nucleotide sequence that has greater than about 60% nucleotide sequence identity, 65%, 70%, 75%, 80%, 85%, 90%, preferably 91%, 92%, 93%, 94%, 95%, 96%, 97%, 98% or 99% or greater nucleotide sequence identity, preferably over a region of over a region of at least about 25, 50, 100, 200, 500, 1000, or more nucleotides, to a nucleotide sequence of or associated with a unigene cluster of Tables 1 or Table 2; (2) bind to antibodies, e.g., polyclonal antibodies, raised against an immunogen comprising an amino acid sequence encoded by a nucleotide sequence of or associated with a unigene cluster of Table 1 or Table 2, and conservatively modified variants thereof; (3) specifically hybridize under stringent hybridization conditions to a nucleic acid sequence, or the complement thereof of Table 1 or Table 2 and conservatively modified variants thereof or (4) have an amino acid sequence that has greater than about 60% amino acid sequence identity, 65%, 70%, 75%, 80%, 85%, 90%, preferably 91%, 92%, 93%, 94%, 95%, 96%, 97%, or greater amino sequence identity, preferably over a region of over a region of at least about 25, 50, 100, 200, 500, 1000, or more amino acid, to an amino acid sequence encoded by a nucleotide sequence of or associated with a unigene cluster of Table 1 or Table 2. A polynucleotide or polypeptide sequence is typically from a mammal including, but not limited to, primate, e.g., human; rodent, e.g., rat, mouse, hamster; cow, pig, horse, sheep, or other mammal. A “colorectal cancer polypeptide” and a “colorectal cancer polynucleotide,” include both naturally occurring or recombinant.

Homology in this context means sequence similarity or identity, with identity being preferred. A preferred comparison for homology purposes is to compare the sequence containing sequencing errors to the correct sequence. This homology will be determined using standard techniques known in the art, including, but not limited to, the local homology algorithm of Smith & Waterman, Adv. Appl. Math. 2:482 (1981), by the homology alignment algorithm of Needleman & Wunsch, J. Mol. Biool. 48:443 (1970), by the search for similarity method of Pearson & Lipman, PNAS USA 85:2444 (1988), by computerized implementations of these algorithms (GAP, BESTFHT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection.

In a preferred embodiment, the sequences which are used to determine sequence identity or similarity are selected from the sequences set forth in Table 1 or Table 2. In one embodiment the sequences utilized herein are those set forth in Table 1 or Table 2. In another embodiment, the sequences are naturally occurring allelic variants of the sequences set forth in Table 1 or Table 2. In another embodiment, the sequences are sequence variants as further described herein.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90,.110, 150, 170, etc.

In one embodiment, the nucleic acid homology is determined through hybridization studies. Thus, for example, nucleic acids which hybridize under high stringency to the nucleic acid sequences which encode the peptides identified in Table 1 or Table 2, or their complements, are considered a colorectal cancer sequence. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide.

In another embodiment, less stringent hybridization conditions are used; for example, moderate or low stringency conditions may be used, as are known in the art; see Maniatis and Ausubel, supra, and Tijssen, supra. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for sec -2 min., an annealing phase lasting 30 sec.-2 min., and an extension phase of about 72° C. for 1-2 min. Protocols and guidelines for low and high stringency amplification reactions are provided, e.g., in Innis et al., PCR Protocols, A Guide to Methods and Applications (1990).

In addition, the colorectal cancer nucleic acid sequences of the invention are fragments of larger genes, i.e. they are nucleic acid segments. “Genes” in this context includes coding regions, non-coding regions, and mixtures of coding and non-coding regions. Accordingly, as will be appreciated by those in the art, using the sequences provided herein, additional sequences of the colorectal cancer genes can be obtained, using techniques well known in the art for cloning either longer sequences or the full length sequences; see Maniatis et al., and Ausubel, et al., supra, hereby expressly incorporated by reference.

An indication that two nucleic acid sequences or polypeptides are substantially identical is that the polypeptide encoded by the first nucleic acid is immunologically cross reactive with the antibodies raised against the polypeptide encoded by the second nucleic acid. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described above. Yet another indication that two nucleic acid sequences are substantially identical is that the same primers can be used to amplify the sequences.

Once the colorectal cancer nucleic acid is identified, it can be cloned and, if necessary, its constituent parts recombined to form the entire colorectal cancer nucleic acid. Once isolated from its natural source, e.g., contained within a plasmid or other vector or excised therefrom as a linear nucleic acid segment, the recombinant colorectal cancer nucleic acid can be further-used as a probe to identify and isolate other colorectal cancer nucleic acids, for example additional coding regions. It can also be used as a “precursor” nucleic acid to make modified or variant colorectal cancer nucleic acids and proteins.

The colorectal cancer nucleic acids of the present invention are used in several ways. In a first embodiment, nucleic acid probes to the colorectal cancer nucleic acids are made and attached to biochips to be used in screening and diagnostic methods, as outlined below, or for administration, for example for gene therapy and/or antisense applications. Alternatively, the colorectal cancer nucleic acids that include coding regions of colorectal cancer proteins can be put into expression vectors for the expression of colorectal cancer proteins, again either for screening purposes or for administration to a patient.

In a preferred embodiment, nucleic acid probes to colorectal cancer nucleic acids (both the nucleic acid sequences encoding peptides outlined in the Table 1 or Table 2 and/or the complements thereof) are made. The nucleic acid probes attached to the biochip are designed to be substantially complementary to the colorectal cancer nucleic acids, i.e. the target sequence (either the target sequence of the sample or to other probe sequences, for example in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally whole genes are not used. In some embodiments, much longer nucleic acids can be used, up to hundreds of bases.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (i.e. have some sequence in common), or separate.

As will be appreciated by those in the art, nucleic acids can be attached or immobilized to a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can be covalent or non-covalent. By “non-covalent binding” and grammatical equivalents herein is meant one or more of either electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant any material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, plastics, etc. In general, the substrates allow optical detection and do not appreciably fluoresce. A preferred substrate is described in copending application entitled Reusable Low Fluorescent Plastic Biochip, U.S. application Ser. No. 09/270,214, filed Mar. 15, 1999, herein incorporated by reference in its entirety.

Generally the substrate is planar, although as will be appreciated by those in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In a preferred embodiment, the surface of the biochip and the probe may be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference). In addition, in some cases, additional linkers, such as alkyl groups (including substituted and heteroalkyl groups) may be used.

In this embodiment, the oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In an additional embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affimetrix GeneChip™ technology.

In a preferred embodiment, colorectal cancer nucleic acids encoding colorectal cancer proteins are used to make a variety of expression vectors to express colorectal cancer proteins which can then be used in screening assays, as described below. The expression vectors may be either self-replicating extrachromosomal vectors or vectors which integrate into a host genome. Generally, these expression vectors include transcriptional and translational regulatory nucleic acid operably linked to the nucleic acid encoding the colorectal cancer protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the colorectal cancer protein; for example, transcriptional and translational regulatory nucleic acid sequences from Bacillus are preferably used to express the colorectal cancer protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

The colorectal cancer proteins of the present invention are produced by culturing a host cell transformed with an expression vector containing nucleic acid encoding a colorectal cancer protein, under the appropriate conditions to induce or cause expression of the colorectal cancer protein. The conditions appropriate for colorectal cancer protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect and animal cells, including mammalian cells. Of particular interest are Drosophila melangaster cells, Saccharomyces cerevisiae and other yeasts, E. coli, Bacillus subtilis, Sf9 cells, C129 cells, 293 cells, Neurospora, BHK, CHO, COS, HeLa cells, THPL cell line (a macrophage cell line) and human cells and cell lines.

In a preferred embodiment, the colorectal cancer proteins are expressed in mammalian cells. Mammalian expression systems are also known in the art, and include retroviral systems. A preferred expression vector system is a retroviral vector system such as is generally described in PCT/US97/01019 and PCT/US97/01048, both of which are hereby expressly incorporated by reference. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, colorectal cancer proteins are expressed in bacterial systems. Bacterial expression systems are well known in the art. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the colorectal cancer protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others.

In one embodiment, colorectal cancer proteins are produced in insect cells. Expression vectors for the transformation of insect cells, and in particular, baculovirus-based expression vectors, are well known in the art.

In a preferred embodiment, colorectal cancer protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

The colorectal cancer protein may also be made as a fusion protein, using techniques well known in the art. Thus, for example, for the creation of monoclonal antibodies, if the desired epitope is small, the colorectal cancer protein may be fused to a carrier protein to form an immunogen. Alternatively, the colorectal cancer protein may be made as a fusion protein to increase expression, or for other reasons. For example, when the colorectal cancer protein is a colorectal cancer peptide, the nucleic acid encoding the peptide may be linked to other nucleic acid for expression purposes.

In one embodiment, the colorectal cancer nucleic acids, proteins and antibodies of the invention are labeled. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the colorectal cancer nucleic acids, proteins and antibodies at any position. For example, the label should be capable of producing, either directly or indirectly, a detectable signal. The detectable moiety may be a radioisotope, such as 3H, 14C, 32P, 35S, or 125I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Accordingly, the present invention also provides colorectal cancer protein sequences. A colorectal cancer protein of the present invention may be identified in several ways. “Protein” in this sense includes proteins, polypeptides, and peptides terms which are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

As will be appreciated by those in the art, the nucleic acid sequences of the invention can be used to generate protein sequences. There are a variety of ways to do this, including cloning the entire gene and verifying its frame and amino acid sequence, or by comparing it to known sequences to search for homology to provide a frame, assuming the colorectal cancer protein has homology to some protein in the database being used. Generally, the nucleic acid sequences are input into a program that will search all three frames for homology. This is done in a preferred embodiment using the following NCBI Advanced BLAST parameters. The program is blastx or blastn. The database is nr. The input data is as “Sequence in FASTA format”. The organism list is “none”. The “expect” is 10; the filter is default. The “descriptions” is 500, the “alignments” is 500, and the “alignment view” is pairwise. The “Query Genetic Codes” is standard (1). The matrix is BLOSUM62; gap existence cost is 11, per residue gap cost is 1; and the lambda ratio is 0.85 default. This results in the generation of a putative protein sequence.

Also included within one embodiment of colorectal cancer proteins are amino acid variants of the naturally occurring sequences, as determined herein. Preferably, the variants are preferably greater than about 75% homologous to the wild-type sequence, more preferably greater than about 80%, even more preferably greater than about 85% and most preferably greater than 90%. In some embodiments the homology will be as high as about 93 to 95 or 98%. As for nucleic acids, homology in this context means sequence similarity or identity, with identity being preferred. This homology will be determined using standard techniques known in the art as are outlined above for the nucleic acid homologies.

Colorectal cancer proteins of the present invention may be shorter or longer than the wild type amino acid sequences. Thus, in a preferred embodiment, included within the definition of colorectal cancer proteins are portions or fragments of the wild type sequences. herein. In addition, as outlined above, the colorectal cancer nucleic acids of the invention may be used to obtain additional coding regions, and thus additional protein sequence, using techniques known in the art.

In a preferred embodiment, the colorectal cancer proteins are derivative or variant colorectal cancer proteins as compared to the wild-type sequence. That is, as outlined more fully below, the derivative colorectal cancer peptide will contain at least one amino acid substitution, deletion or insertion, with amino acid substitutions being particularly preferred. The amino acid substitution, insertion or deletion may occur at any residue within the colorectal cancer peptide.

Also included in an embodiment of colorectal cancer proteins of the present invention are amino acid sequence variants. These variants fall into one or more of three classes: substitutional, insertional or deletional variants. These variants ordinarily are prepared by site specific mutagenesis of nucleotides in the DNA encoding the colorectal cancer protein, using cassette or PCR mutagenesis or other techniques well known in the art, to produce DNA encoding the variant, and thereafter expressing the DNA in recombinant cell culture as outlined above. However, variant colorectal cancer protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the colorectal cancer protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed colorectal cancer variants screened for the optimal combination of desired activity. Techniques for making substitution mutations at predetermined sites in DNA having a known sequence are well known, for example, M13 primer mutagenesis and PCR mutagenesis. Screening of the mutants is done using assays of colorectal cancer protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the colorectal cancer protein are desired, substitutions are generally made in accordance with the following chart:

CHART I

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the colorectal cancer proteins as needed. Alternatively, the variant may be designed such that the biological activity of the colorectal cancer protein is altered. For example, glycosylation sites may be altered or removed.

Covalent modifications of colorectal cancer polypeptides are included within the scope of this invention. One type of covalent modification includes reacting targeted amino acid residues of a colorectal cancer polypeptide with an organic derivatizing agent that is capable of reacting with selected side chains or the N-or C-terminal residues of a colorectal cancer polypeptide. Derivatization with bifunctional agents is useful, for instance, for crosslinking colorectal cancer to a water-insoluble support matrix or surface for use in the method for purifying anti-colorectal cancer antibodies or screening assays, as is more fully described below. Commonly used crosslinking agents include, e.g., 1,1-bis(diazo-acetyl)-2-phenylethane, glutaraldehyde, N-hydroxy-succinimide esters, for example, esters with 4-azido-salicylic acid, homobifunctional imidoesters, including disuccinimidyl esters such as 3,3′-dithiobis-(succinimidyl-propionate), bifunctional maleimides such as bis-N-maleimido-1,8-octane and agents such as methyl-3-[(p-azidophenyl)-dithio]pro-pioimi-date.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the α-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the colorectal cancer polypeptide included within the scope of this invention comprises altering the native glycosylation pattern of the polypeptide. “Altering the native glycosylation pattern” is intended for purposes herein to mean deleting one or more carbohydrate moieties found in native sequence colorectal cancer polypeptide, and/or adding one or more glycosylation sites that are not present in the native sequence colorectal cancer polypeptide.

Addition of glycosylation sites to colorectal cancer polypeptides may be accomplished by altering the amino acid sequence thereof. The alteration may be made, for example, by the addition of, or substitution by, one or more serine or threonine residues to the native sequence colorectal cancer polypeptide (for O-linked glycosylation sites). The colorectal cancer amino acid sequence may optionally be altered through changes at the DNA level, particularly by mutating the DNA encoding the colorectal cancer polypeptide at preselected bases such that codons are generated that will translate into the desired amino acids.

Another means of increasing the number of carbohydrate moieties on the colorectal cancer polypeptide is by chemical or enzymatic coupling of glycosides to the polypeptide. Such methods are described in the art, e.g., in WO 87/05330 published Sep. 11, 1987, and in Aplin and Wriston, colorectal cancer Crit. Rev. Biochem., pp. 259-306 (1981).

Removal of carbohydrate moieties present on the colorectal cancer polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Another type of covalent modification of colorectal cancer comprises linking the colorectal cancer polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

colorectal cancer polypeptides of the present invention may also be modified in a way to form chimeric molecules comprising a colorectal cancer polypeptide fused to another, heterologous polypeptide or amino acid sequence. In one embodiment, such a chimeric molecule comprises a fusion of a colorectal cancer polypeptide with a tag polypeptide which provides an epitope to which an anti-tag antibody can selectively bind. The epitope tag is generally placed at the amino-or carboxyl-terminus of the colorectal cancer polypeptide. The presence of such epitope-tagged forms of a colorectal cancer polypeptide can be detected using an antibody against the tag polypeptide. Also, provision of the epitope tag enables the colorectal cancer polypeptide to be readily purified by affinity purification using an anti-tag antibody or another type of affinity matrix that binds to the epitope tag. In an alternative embodiment, the chimeric molecule may comprise a fusion of a colorectal cancer polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

Also included with the definition of colorectal cancer protein in one embodiment are other colorectal cancer proteins of the colorectal cancer family, and colorectal cancer proteins from other organisms, which are cloned and expressed as outlined below. Thus, probe or degenerate polymerase chain reaction (PCR) primer sequences may be used to find other related colorectal cancer proteins from humans or other organisms. As will be appreciated by those in the art, particularly useful probe and/or PCR primer sequences include the unique areas of the colorectal cancer nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art.

In addition, as is outlined herein, colorectal cancer proteins can be made that are longer than those depicted in the Table 1 or Table 2 for example, by the elucidation of additional sequences, the addition of epitope or purification tags, the addition of other fusion sequences, etc.

Colorectal cancer proteins may also be identified as being encoded by colorectal cancer nucleic acids. Thus, colorectal cancer proteins are encoded by nucleic acids that will hybridize to the sequences of the sequence listings, or their complements, as outlined herein.

In a preferred embodiment, when the colorectal cancer protein is to be used to generate antibodies, for example for immunotherapy, the colorectal cancer protein should share at least one epitope or determinant with the full length protein. By “epitope” or “determinant” herein is meant a portion of a protein which will generate and/or bind an antibody or T-cell receptor in the context of MHC. Thus, in most instances, antibodies made to a smaller colorectal cancer protein will be able to bind to the full length protein. In a preferred embodiment, the epitope is unique; that is, antibodies generated to a unique epitope show little or no cross-reactivity. In a preferred embodiment, the epitope is selected from a peptide encoded by a nucleic acid of Table 1. In another preferred embodiment, the epitope is selected from the CBF9 peptide sequence shown in Table 2.

In one embodiment, the term “antibody” includes antibody fragments, as are known in the art, including Fab, Fab2, single chain antibodies (Fv for example), chimeric antibodies, etc., either produced by the modification of whole antibodies or those synthesized de novo using recombinant DNA technologies.

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the CBF9 peptide of Table 2, or a peptide encoded by a nucleic acid of Table 1 or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include the CBF9 polypeptide or a peptide encoded by a nucleic acid of Table 1 or a fragment thereof or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are monoclonal, preferably human or humanized, antibodies that have binding specificities for at least two different antigens. In the present case, one of the binding specificities is for a colorectal cancer protein or a fragment thereof, the other one is for any other antigen, and preferably for a cell-surface protein or receptor or receptor subunit, preferably one that is tumor specific.

In a preferred embodiment, the antibodies to colorectal cancer are capable of reducing or eliminating the biological function of colorectal cancer, as is described below. That is, the addition of anti-colorectal cancer antibodies (either polyclonal or preferably monoclonal) to colorectal cancer (or cells containing colorectal cancer) may reduce or eliminate the colorectal cancer activity. Generally, at least a 25% decrease in activity is preferred, with at least about 50% being particularly preferred and about a 95-100% decrease being especially preferred.

In a preferred embodiment the antibodies to the colorectal cancer proteins are humanized antibodies. Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

By immunotherapy is meant treatment of colorectal cancer with an antibody raised against colorectal cancer proteins. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen.

In a preferred embodiment the colorectal cancer proteins against which antibodies are raised are secreted proteins as described above. Without being bound by theory, antibodies used for treatment, bind and prevent the secreted protein from binding to its receptor, thereby inactivating the secreted colorectal cancer protein.

In another preferred embodiment, the colorectal cancer protein to which antibodies are raised is a transmembrane protein. Without being bound by theory, antibodies used for treatment, bind the extracellular domain of the colorectal cancer protein and prevent it from binding to other proteins, such as circulating ligands or cell-associated molecules. The antibody may cause down-regulation of the transmembrane colorectal cancer protein. As will be appreciated by one of ordinary skill in the art, the antibody may be a competitive, non-competitive or uncompetitive inhibitor of protein binding to the extracellular domain of the colorectal cancer protein. The antibody is also an antagonist of the colorectal cancer protein. Further, the antibody prevents activation of the transmembrane colorectal cancer protein. In one aspect, when the antibody prevents the binding of other molecules to the colorectal cancer protein, the antibody prevents growth of the cell. The antibody also sensitizes the cell to cytotoxic agents, including, but not limited to TNF-α TNF-β, IL-1, INF-γ and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity. Thus, colorectal cancer is treated by administering to a patient antibodies directed against the transmembrane colorectal cancer protein.

In another preferred embodiment, the antibody is conjugated to a therapeutic moiety. In one aspect the therapeutic moiety is a small molecule that modulates the activity of the colorectal cancer protein. In another aspect the therapeutic moiety modulates the activity of molecules associated with or in close proximity to the colorectal cancer protein. The therapeutic moiety may inhibit enzymatic activity such as protease or protein kinase activity associated with colorectal cancer.

In a preferred embodiment, the therapeutic moiety may also be a cytotoxic agent. In this method, targeting the cytotoxic agent to tumor tissue or cells, results in a reduction in the number of afflicted cells, thereby reducing symptoms associated with colorectal cancer. Cytotoxic agents are numerous and varied and include, but are not limited to, cytotoxic drugs or toxins or active fragments of such toxins. Suitable toxins and their corresponding fragments include diptheria A chain, exotoxin A chain, ricin A chain, abrin A chain, curcin, crotin, phenomycin, enomycin and the like. Cytotoxic agents also include radiochemicals made by conjugating radioisotopes to antibodies raised against colorectal cancer proteins, or binding of a radionuclide to a chelating agent that has been covalently attached to the antibody. Targeting the therapeutic moiety to transmembrane colorectal cancer proteins not only serves to increase the local concentration of therapeutic moiety in the colorectal cancer afflicted area, but also serves to reduce deleterious side effects that may be associated with the therapeutic moiety.

In another preferred embodiment, the colorectal cancer protein against which the antibodies are raised is an intracellular protein. In this case, the antibody may be conjugated to a protein which facilitates entry into the cell. In one case, the antibody enters the cell by endocytosis. In another embodiment, a nucleic acid encoding the antibody is administered to the individual or cell. Moreover, wherein the colorectal cancer protein can be targeted within a cell, i.e., the nucleus, an antibody thereto contains a signal for that target localization, i.e., a nuclear localization signal.

The colorectal cancer antibodies of the invention specifically bind to colorectal cancer proteins. By “specifically bind” herein is meant that the antibodies bind to the protein with a binding constant in the range of at least 10⁻⁴10⁻⁶ M⁻¹, with a preferred range being 10⁻⁷-10⁻⁹ MN⁻¹.

In a preferred embodiment, the colorectal cancer protein is purified or isolated after expression. Colorectal cancer proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the colorectal cancer protein may be purified using a standard anti-colorectal cancer antibody column. Ultrafiltration and diafiltration techniques, in conjunction with protein concentration, are also useful. For general guidance in suitable purification techniques, see Scopes, R., Protein Purification, Springer-Verlag, N.Y. (1982). The degree of purification necessary will vary depending on the use of the colorectal cancer protein. In some instances no purification will be necessary.

Once expressed and purified if necessary, the colorectal cancer proteins and nucleic acids are useful in a number of applications.

In one aspect, the expression levels of genes are determined for different cellular states in the colorectal cancer phenotype; that is, the expression levels of genes in normal colon tissue and in colorectal cancer tissue (and in some cases, for varying severities of colorectal cancer that relate to prognosis, as outlined below) are evaluated to provide expression profiles. An expression profile of a particular cell state or point of development is essentially a “fingerprint” of the state; while two states may have any particular gene similarly expressed, the evaluation of a number of genes simultaneously allows the generation of a gene expression profile that is unique to the state of the cell. By comparing expression profiles of cells in different states, information regarding which genes are important (including both up- and down-regulation of genes) in each of these states is obtained. Then, diagnosis may be done or confirmed: does tissue from a particular patient have the gene expression profile of normal or colorectal cancer tissue.

“Differential expression,” or grammatical equivalents as used herein, refers to both qualitative as well as quantitative differences in the gene' temporal and/or cellular expression patterns within and among the cells. Thus, a differentially expressed gene can qualitatively have its expression altered, including an activation or inactivation, in, for example, normal versus colorectal cancer tissue. That is, genes may be turned on or turned off in a particular state, relative to another state. As is apparent to the skilled artisan, any comparison of two or more states can be made. Such a qualitatively regulated gene will exhibit an expression pattern within a state or cell type which is detectable by standard techniques in one such state or cell type, but is not detectable in both. Alternatively, the determination is quantitative in that expression is increased or decreased; that is, the expression of the gene is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays, Lockhart, Nature Biotechnology, 14:1675-1680 (1996), hereby expressly incorporated by reference. Other techniques include, but are not limited to, quantitative reverse transcriptase PCR, Northern analysis and RNase protection. As outlined above, preferably the change in expression (i.e. upregulation or downregulation) is at least about 50%, more preferably at least about 100%, more preferably at least about 150%, more preferably, at least about 200%, with from 300 to at least 1000% being especially preferred.

As will be appreciated by those in the art, this may be done by evaluation at either the gene transcript, or the protein level; that is, the amount of gene expression may be monitored using nucleic acid probes to the DNA or RNA equivalent of the gene transcript, and the quantification of gene expression levels, or, alternatively, the final gene product itself (protein) can be monitored, for example through the use of antibodies to the colorectal cancer protein and standard immunoassays (ELISAs, etc.) or other techniques, including mass spectroscopy assays, 2D gel electrophoresis assays, etc. Thus, the proteins corresponding to colorectal cancer genes, i.e. those identified as being important in a colorectal cancer phenotype, can be evaluated in a colorectal cancer diagnostic test.

In a preferred embodiment, gene expression monitoring is done and a number of genes, i.e. an expression profile, is monitored simultaneously, although multiple protein expression monitoring can be done as well. Similarly, these assays may be done on an individual basis as well.

In this embodiment, the colorectal cancer nucleic acid probes are attached to biochips as outlined herein for the detection and quantification of colorectal cancer sequences in a particular cell. The assays are further described below in the example.

In a preferred embodiment nucleic acids encoding the colorectal cancer protein are detected. Although DNA or RNA encoding the colorectal cancer protein may be detected, of particular interest are methods wherein the mRNA encoding a colorectal cancer protein is detected. The presence of mRNA in a sample is an indication that the colorectal cancer gene has been transcribed to form the mRNA, and suggests that the protein is expressed. Probes to detect the mRNA can be any nucleotide/deoxynucleotide probe that is complementary to and base pairs with the mRNA and includes but is not limited to oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a colorectal cancer protein is detected by binding the digoxygenin with an anti-digoxygenin secondary antibody and developed with nitro blue tetrazolium and 5-bromo-4-chloro-3-indoyl phosphate.

In a preferred embodiment, any of the three classes of proteins as described herein (secreted, transmembrane or intracellular proteins) are used in diagnostic assays. The colorectal cancer proteins, antibodies, nucleic acids, modified proteins and cells containing colorectal cancer sequences are used in diagnostic assays. This can be done on an individual gene or corresponding polypeptide level. In a preferred embodiment, the expression profiles are used, preferably in conjunction with high throughput screening techniques to allow monitoring for expression profile genes and/or corresponding polypeptides.

As described and defined herein, colorectal cancer proteins, including intracellular, transmembrane or secreted proteins, find use as markers of colorectal cancer. Detection of these proteins in putative colorectal cancer tissue or patients allows for a determination or diagnosis of colorectal cancer. Numerous methods known to those of ordinary skill in the art find use in detecting colorectal cancer. In one embodiment, antibodies are used to detect colorectal cancer proteins. A preferred method separates proteins from a sample or patient by electrophoresis on a gel (typically a denaturing and reducing protein gel, but may be any other type of gel including isoelectric focusing gels and the like). Following separation of proteins, the colorectal cancer protein is detected by immunoblotting with antibodies raised against the colorectal cancer protein. Methods of immunoblotting are well known to those of ordinary skill in the art.

In another preferred method, antibodies to the colorectal cancer protein find use in in situ imaging techniques. In this method cells are contacted with from one to many antibodies to the colorectal cancer protein(s). Following washing to remove non-specific antibody binding, the presence of the antibody or antibodies is detected. In one embodiment the antibody is detected by incubating with a secondary antibody that contains a detectable label. In another method the primary antibody to the colorectal cancer protein(s) contains a detectable label. In another preferred embodiment each one of multiple primary antibodies contains a distinct and detectable label. This method finds particular use in simultaneous screening for a plurality of colorectal cancer proteins. As will be appreciated by one of ordinary skill in the art, numerous other histological imaging techniques are useful in the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing colorectal cancer from blood samples. As previously described, certain colorectal cancer proteins are secreted/circulating molecules. Blood samples, therefore, are useful as samples to be probed or tested for the presence of secreted colorectal cancer proteins. Antibodies can be used to detect the colorectal cancer by any of the previously described immunoassay techniques including ELISA, immunoblotting (Western blotting), immunoprecipitation, BIACORE technology and the like, as will be appreciated by one of ordinary skill in the art.

In a preferred embodiment, in situ hybridization of labeled colorectal cancer nucleic acid probes to tissue arrays is done. For example, arrays of tissue samples, including colorectal cancer tissue and/or normal tissue, are made. In situ hybridization as is known in the art can then be done.

It is understood that when comparing the fingerprints between an individual and a standard, the skilled artisan can make a diagnosis as well as a prognosis. It is further understood that the genes which indicate the diagnosis may differ from those which indicate the prognosis.

In a preferred embodiment, the colorectal cancer proteins, antibodies, nucleic acids, modified proteins and cells containing colorectal cancer sequences are used in prognosis assays. As above, gene expression profiles can be generated that correlate to colorectal cancer severity, in terms of long term prognosis. Again, this may be done on either a protein or gene level, with the use of genes being preferred. As above, the colorectal cancer probes are attached to biochips for the detection and quantification of colorectal cancer sequences in a tissue or patient. The assays proceed as outlined for diagnosis.

In a preferred embodiment, any of the three classes of proteins as described herein are used in drug screening assays. The colorectal cancer proteins, antibodies, nucleic acids, modified proteins and cells containing colorectal cancer sequences are used in drug screening assays or by evaluating the effect of drug candidates on a “gene expression profile” or expression profile of polypeptides. In a preferred embodiment, the expression profiles are used, preferably in conjunction with high throughput screening techniques to allow monitoring for expression profile genes after treatment with a candidate agent, Zlokarnik, et al., Science 279, 84-8 (1998), Heid, 1996 #69.

In a preferred embodiment, the colorectal cancer proteins, antibodies, nucleic acids, modified proteins and cells containing the native or modified colorectal cancer proteins are used in screening assays. That is, the present invention provides novel methods for screening for compositions which modulate the colorectal cancer phenotype. As above, this can be done on an individual gene level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the differentially expressed genes herein, a variety of assays may be executed. In a preferred embodiment, assays may be run on an individual gene or protein level. That is, having identified a particular gene as up regulated in colorectal cancer, candidate bioactive agents may be screened to modulate this gene's response; preferably to down regulate the gene, although in some circumstances to up regulate the gene. “Modulation” thus includes both an increase and a decrease in gene expression. The preferred amount of modulation will depend on the original change of the gene expression in normal versus tumor tissue, with changes of at least 10%, preferably 50%, more preferably 100-300%, and in some embodiments 300-1000% or greater. Thus, if a gene exhibits a 4 fold increase in tumor compared to normal tissue, a decrease of about four fold is desired; a 10 fold decrease in tumor compared to normal tissue gives a 10 fold increase in expression for a candidate agent is desired.

As will be appreciated by those in the art, this may be done by evaluation at either the gene or the protein level; that is, the amount of gene expression may be monitored using nucleic acid probes and the quantification of gene expression levels, or, alternatively, the gene product itself can be monitored, for example through the use of antibodies to the colorectal cancer protein and standard immunoassays.

In a preferred embodiment, gene expression monitoring is done and a number of genes, i.e. an expression profile, is monitored simultaneously, although multiple protein expression monitoring can be done as well.

In this embodiment, the colorectal cancer nucleic acid probes are attached to biochips as outlined herein for the detection and quantification of colorectal cancer sequences in a particular cell. The assays are further described below.

Generally, in a preferred embodiment, a candidate bioactive agent is added to the cells prior to analysis. Moreover, screens are provided to identify a candidate bioactive agent which modulates colorectal cancer, modulates colorectal cancer proteins, binds to a colorectal cancer protein, or interferes between the binding of a colorectal cancer protein and an antibody.

The term “candidate bioactive agent” or “drug candidate” or grammatical equivalents as used herein describes any molecule, e.g., protein, oligopeptide, small organic molecule, polysaccharide, polynucleotide, etc., to be tested for bioactive agents that are capable of directly or indirectly altering either the colorectal cancer phenotype or the expression of a colorectal cancer sequence, including both nucleic acid sequences and protein sequences. In preferred embodiments, the bioactive agents modulate the expression profiles, or expression profile nucleic acids or proteins provided herein. In a particularly preferred embodiment, the candidate agent suppresses a colorectal cancer phenotype, for example to a normal colon tissue fingerprint. Similarly, the candidate agent preferably suppresses a severe colorectal cancer phenotype. Generally a plurality of assay mixtures are run in parallel with different agent concentrations to obtain a differential response to the various concentrations. Typically, one of these concentrations serves as a negative control, i.e., at zero concentration or below the level of detection.

In one aspect, a candidate agent will neutralize the effect of a colorectal cancer protein. By “neutralize” is meant that activity of a protein is either inhibited or counter acted against so as to have substantially no effect on a cell.

Candidate agents encompass numerous chemical classes, though typically they are organic molecules, preferably small organic compounds having a molecular weight of more than 100 and less than about 2,500 daltons. Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the methods of the invention. Particularly preferred in this embodiment are libraries of bacterial, fungal, viral, and mammalian proteins, with the latter being preferred, and human proteins being especially preferred.

In a preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

In a preferred embodiment, the candidate bioactive agents are nucleic acids, as defined above.

As described above generally for proteins, nucleic acid candidate bioactive agents may be naturally occurring nucleic acids, random nucleic acids, or “biased” random nucleic acids. For example, digests of procaryotic or eucaryotic genomes may be used as is outlined above for proteins.

In a preferred embodiment, the candidate bioactive agents are organic chemical moieties, a wide variety of which are available in the literature.

After the candidate agent has been added and the cells allowed to incubate for some period of time, the sample containing the target sequences to be analyzed is added to the biochip. If required, the target sequence is prepared using known techniques. For example, the sample may be treated to lyse the cells, using known lysis buffers, electroporation, etc., with purification and/or amplification such as PCR occurring as needed, as will be appreciated by those in the art. For example, an in vitro transcription with labels covalently attached to the nucleosides is done. Generally, the nucleic acids are labeled with biotin-FITC or PE, or with cy3 or cy5.

In a preferred embodiment, the target sequence is labeled with, for example, a fluorescent, a chemiluminescent, a chemical, or a radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. As known in the art, unbound labeled streptavidin is removed prior to analysis.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions which allows formation of the label probe hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents may be included in the assays. These include reagents like salts, buffers, neutral proteins, e.g. albumin, detergents, etc. which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target.

Once the assay is run, the data is analyzed to determine the expression levels, and changes in expression levels as between states, of individual genes, forming a gene expression profile.

The screens are done to identify drugs or bioactive agents that modulate the colorectal cancer phenotype. Specifically, there are several types of screens that can be run. A preferred embodiment is in the screening of candidate agents that can induce or suppress a particular expression profile, thus preferably generating the associated phenotype. That is, candidate agents that can mimic or produce an expression profile in colorectal cancer similar to the expression profile of normal colon tissue is expected to result in a suppression of the colorectal cancer phenotype. Thus, in this embodiment, mimicking an expression profile, or changing one profile to another, is the goal.

In a preferred embodiment, as for the diagnosis and prognosis applications, having identified the differentially expressed genes important in any one state, screens can be run to alter the expression of the genes individually. That is, screening for modulation of regulation of expression of a single gene can be done; that is, rather than try to mimic all or part of an expression profile, screening for regulation of individual genes can be done. Thus, for example, particularly in the case of target genes whose presence or absence is unique between two states, screening is done for modulators of the target gene expression.

In a preferred embodiment, screening is done to alter the biological function of the expression product of the differentially expressed gene. Again, having identified the importance of a gene in a particular state, screening for agents that bind and/or modulate the biological activity of the gene product can be run as is more fully outlined below.

Thus, screening of candidate agents that modulate the colorectal cancer phenotype either at the gene expression level or the protein level can be done.

In addition screens can be done for novel genes that are induced in response to a candidate agent. After identifying a candidate agent based upon its ability to suppress a colorectal cancer expression pattern leading to a normal expression pattern, or modulate a single colorectal cancer gene expression profile so as to mimic the expression of the gene from normal tissue, a screen as described above can be performed to identify genes that are specifically modulated in response to the agent. Comparing expression profiles between normal tissue and agent treated colorectal cancer tissue reveals genes that are not expressed in normal tissue or colorectal cancer tissue, but are expressed in agent treated tissue. These agent specific sequences can be identified and used by any of the methods described herein for colorectal cancer genes or proteins. In particular these sequences and the proteins they encode find use in marking or identifying agent treated cells. In addition, antibodies can be raised against the agent induced proteins and used to target novel therapeutics to the treated colorectal cancer tissue sample.

Thus, in one embodiment, a candidate agent is administered to a population of colorectal cancer cells, that thus has an associated colorectal cancer expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e. a peptide) may be put into a viral construct such as a retroviral construct and added to the cell, such that expression of the peptide agent is accomplished; see PCT US97/01019, hereby expressly incorporated by reference.

Once the candidate agent has been administered to the cells, the cells can be washed if desired and are allowed to incubate under preferably physiological conditions for some period of time. The cells are then harvested and a new gene expression profile is generated, as outlined herein.

Thus, for example, colorectal cancer tissue may be screened for agents that reduce or suppress the colorectal cancer phenotype. A change in at least one gene of the expression profile indicates that the agent has an effect on colorectal cancer activity. By defining such a signature for the colorectal cancer phenotype, screens for new drugs that alter the phenotype can be devised. With this approach, the drug target need not be known and need not be represented in the original expression screening platform, nor does the level of transcript for the target protein need to change.

In a preferred embodiment, as outlined above, screens may be done on individual genes and gene products (proteins). That is, having identified a particular differentially expressed gene as important in a particular state, screening of modulators of either the expression of the gene or the gene product itself can be done. The gene products of differentially expressed genes are sometimes referred to herein as “colorectal cancer modulator proteins”. The colorectal cancer modulator protein may be a fragment, or alternatively, be the full length protein to a fragment shown herein. Preferably, the colorectal cancer modulator protein is a fragment of approximately 14 to 24 amino acids long. More preferably the fragment is a soluble fragment.

In a preferred embodiment, the fragment is charged and from the c-terminus. In one embodiment, the c-terminus of the fragment is kept as a free acid and the n-terminus is a free amine to aid in coupling, i.e., to cysteine. In another embodiment, the fragment is an internal peptide overlapping hydrophilic stretch the protein. In a preferred embodiment, the termini is blocked. In another preferred embodiment, the fragment is a novel fragment from the N-terminal. In one embodiment, the fragment excludes sequence outside of the N-terminal, in another embodiment, the fragment includes at least a portion of the N-terminal. “N-terminal” is used interchangeably herein with “N-terminus” which is further described above.

In one embodiment the colorectal cancer proteins are conjugated to an immunogenic agent as discussed herein. In one embodiment the colorectal cancer protein is conjugated to BSA.

Thus, in a preferred embodiment, screening for modulators of expression of specific genes can be done. This will be done as outlined above, but in general the expression of only one or a few genes are evaluated.

In a preferred embodiment, screens are designed to first find candidate agents that can bind to differentially expressed proteins, and then these agents may be used in assays that evaluate the ability of the candidate agent to modulate differentially expressed activity. Thus, as will be appreciated by those in the art, there are a number of different assays which may be run; binding assays and activity assays.

In a preferred embodiment, binding assays are done. In general, purified or isolated gene product is used; that is, the gene products of one or more differentially expressed nucleic acids are made. In general, this is done as is known in the art. For example, antibodies are generated to the protein gene products, and standard immunoassays are run to determine the amount of protein present. Alternatively, cells comprising the colorectal cancer proteins can be used in the assays.

Thus, in a preferred embodiment, the methods comprise combining a colorectal cancer protein and a candidate bioactive agent, and determining the binding of the candidate agent to the colorectal cancer protein. Preferred embodiments utilize the human colorectal cancer protein, although other mammalian proteins may also be used, for example for the development of animal models of human disease. In some embodiments, as outlined herein, variant or derivative colorectal cancer proteins may be used.

Generally, in a preferred embodiment of the methods herein, the colorectal cancer protein or the candidate agent is non-diffusably bound to an insoluble support having isolated sample receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, teflon, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the colorectal cancer protein is bound to the support, and a candidate bioactive agent is added to the assay. Alternatively, the candidate agent is bound to the support and the colorectal cancer protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the candidate bioactive agent to the colorectal cancer protein may be done in a number of ways. In a preferred embodiment, the candidate bioactive agent is labeled, and binding determined directly. For example, this may be done by attaching all or a portion of the colorectal cancer protein to a solid support, adding a labeled candidate agent (for example a fluorescent label), washing off excess reagent, and determining whether the label is present on the solid support. Various blocking and washing steps may be utilized as is known in the art.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the proteins (or proteinaceous candidate agents) may be labeled at tyrosine positions using 125I, or with fluorophores. Alternatively, more than one component may be labeled with different labels; using ¹²⁵I for the proteins, for example, and a fluorophor for the candidate agents.

In a preferred embodiment, the binding of the candidate bioactive agent is determined through the use of competitive binding assays. In this embodiment, the competitor is a binding moiety known to bind to the target molecule (i.e. colorectal cancer), such as an antibody, peptide, binding partner, ligand, etc. Under certain circumstances, there may be competitive binding as between the bioactive agent and the binding moiety, with the binding moiety displacing the bioactive agent.

In one embodiment, the candidate bioactive agent is labeled. Either the candidate bioactive agent, or the competitor, or both, is added first to the protein for a time sufficient to allow binding, if present. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate bioactive agent. Displacement of the competitor is an indication that the candidate bioactive agent is binding to the colorectal cancer protein and thus is capable of binding to, and potentially modulating, the activity of the colorectal cancer protein. In this embodiment, either component can be labeled. Thus, for example, if the competitor is labeled, the presence of label in the wash solution indicates displacement by the agent. Alternatively, if the candidate bioactive agent is labeled, the presence of the label on the support indicates displacement.

In an alternative embodiment, the candidate bioactive agent is added first, with incubation and washing, followed by the competitor. The absence of binding by the competitor may indicate that the bioactive agent is bound to the colorectal cancer protein with a higher affinity. Thus, if the candidate bioactive agent is labeled, the presence of the label on the support, coupled with a lack of competitor binding, may indicate that the candidate agent is capable of binding to the colorectal cancer protein.

In a preferred embodiment, the methods comprise differential screening to identity bioactive agents that are capable of modulating the activity of the colorectal cancer proteins. In this embodiment, the methods comprise combining a colorectal cancer protein and a competitor in a first sample. A second sample comprises a candidate bioactive agent, a colorectal cancer protein and a competitor. The binding of the competitor is determined for both samples, and a change, or difference in binding between the two samples indicates the presence of an agent capable of binding to the colorectal cancer protein and potentially modulating its activity. That is, if the binding of the competitor is different in the second sample relative to the first sample, the agent is capable of binding to the colorectal cancer protein.

Alternatively, a preferred embodiment utilizes differential screening to identify drug candidates that bind to the native colorectal cancer protein, but cannot bind to modified colorectal cancer proteins. The structure of the colorectal cancer protein may be modeled, and used in rational drug design to synthesize agents that interact with that site. Drug candidates that affect colorectal cancer bioactivity are also identified by screening drugs for the ability to either enhance or reduce the activity of the protein.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc. which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Screening for agents that modulate the activity of colorectal cancer proteins may also be done. In a preferred embodiment, methods for screening for a bioactive agent capable of modulating the activity of colorectal cancer proteins comprise the steps of adding a candidate bioactive agent to a sample of colorectal cancer proteins, as above, and determining an alteration in the biological activity of colorectal cancer proteins. “Modulating the activity of colorectal cancer” includes an increase in activity, a decrease in activity, or a change in the type or kind of activity present. Thus, in this embodiment, the candidate agent should both bind to colorectal cancer proteins (although this may not be necessary), and alter its biological or biochemical activity as defined herein. The methods include both in vitro screening methods, as are generally outlined above, and in vivo screening of cells for alterations in the presence, distribution, activity or amount of colorectal cancer proteins.

Thus, in this embodiment, the methods comprise combining a colorectal cancer sample and a candidate bioactive agent, and evaluating the effect on colorectal cancer activity. By “colorectal cancer activity” or grammatical equivalents herein is meant one of the colorectal cancer 's biological activities, including, but not limited to, cell division, preferably in colon tissue, cell proliferation, tumor growth, transformation of cells. In one embodiment, colorectal cancer activity includes activation of a gene identified by a nucleic acid of Table 1. An inhibitor of colorectal cancer activity is the inhibition of any one or more colorectal cancer activities.

In a preferred embodiment, the activity of the colorectal cancer protein is increased; in another preferred embodiment, the activity of the colorectal cancer protein is decreased. Thus, bioactive agents that are antagonists are preferred in some embodiments, and bioactive agents that are agonists may be preferred in other embodiments.

In a preferred embodiment, the invention provides methods for screening for bioactive agents capable of modulating the activity of a colorectal cancer protein. The methods comprise adding a candidate bioactive agent, as defined above, to a cell comprising colorectal cancer proteins. Preferred cell types include almost any cell. The cells contain a recombinant nucleic acid that encodes a colorectal cancer protein. In a preferred embodiment, a library of candidate agents are tested on a plurality of cells.

In one aspect, the assays are evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, bioactive agents are identified. Compounds with pharmacological activity are able to enhance or interfere with the activity of the colorectal cancer protein. In one embodiment, “colorectal cancer protein activity” as used herein includes at least one of the following: colorectal cancer activity, binding to the colorectal cancer protein, activation of the colorectal cancer protein or activation of substrates of the colorectal cancer protein by the colorectal cancer protein. In one embodiment, colorectal cancer activity is defined as the unregulated proliferation of colon tissue, or the growth of cancer in colon tissue. In one aspect, colorectal cancer activity as defined herein is related to the activity of the colorectal cancer protein in the upregulation of the colorectal cancer protein in colon cancer tissue.

In another embodiment, colorectal cancer protein activity includes at least one of the following: colorectal cancer activity, binding to the CBF9 nucleic acid or poly peptide of Table 2 or binding to a nucleic acid of Table 1, or a peptide encoded by a nucleic acid of Table 1 or activation of substrates of the gene products identified by a nucleic acid of Table 1 or substrates of CBF9, which is shown in Table 2. In one aspect, colorectal cancer activity as defined herein is related to the activity of genes defined by the nucleic acids of Table 1 or of CBF9 as defined in Table 2, in colon cancer tissue.

In one embodiment, a method of inhibiting colon cancer cell division is provided. The method comprises administration of a colorectal cancer inhibitor.

In another embodiment, a method of inhibiting tumor growth is provided. The method comprises administration of a colorectal cancer inhibitor.

In a further embodiment, methods of treating cells or individuals with cancer are provided. The method comprises administration of a colorectal cancer inhibitor.

In one embodiment, a colorectal cancer inhibitor is an antibody as discussed above. In another embodiment, the colorectal cancer inhibitor is an antisense molecule. Antisense molecules as used herein include antisense or sense oligonucleotides comprising a singe-stranded nucleic acid sequence (either RNA or DNA) capable of binding to target mRNA (sense) or DNA (antisense) sequences for colorectal cancer molecules. A preferred antisense molecule is for the colorectal cancer sequences referenced in Table 1 or Table 2, or for a ligand or activator thereof. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Antisense molecules may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a sense or an antisense oligonucleotide may be introduced into a cell containing the target nucleic acid sequence by formation of an oligonucleotide-lipid complex, as described in WO 90/10448. It is understood that the use of antisense molecules or knock out and knock in models may also be used in screening assays as discussed above, in addition to methods of treatment.

The compounds having the desired pharmacological activity may be administered in a physiologically acceptable carrier to a host, as previously described. The agents may be administered in a variety of ways, orally, parenterally e.g., subcutaneously, intraperitoneally, intravascularly, etc. Depending upon the manner of introduction, the compounds may be formulated in a variety of ways. The concentration of therapeutically active compound in the formulation may vary from about 0.1-100 wt. %. The agents may be administered alone or in combination with other treatments, i.e., radiation.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

Without being bound by theory, it appears that the various colorectal cancer sequences are important in colorectal cancer. Accordingly, disorders based on mutant or variant colorectal cancer genes may be determined. In one embodiment, the invention provides methods for identifying cells containing variant colorectal cancer genes comprising determining all or part of the sequence of at least one endogeneous colorectal cancer genes in a cell. As will be appreciated by those in the art, this may be done using any number of sequencing techniques. In a preferred embodiment, the invention provides methods of identifying the colorectal cancer genotype of an individual comprising determining all or part of the sequence of at least one colorectal cancer gene of the individual. This is generally done in at least one tissue of the individual, and may include the evaluation of a number of tissues or different samples of the same tissue. The method may include comparing the sequence of the sequenced colorectal cancer gene to a known colorectal cancer gene, i.e. a wild-type gene.

The sequence of all or part of the colorectal cancer gene can then be compared to the sequence of a known colorectal cancer gene to determine if any differences exist. This can be done using any number of known homology programs, such as Bestfit, etc. In a preferred embodiment, the presence of a a difference in the sequence between the colorectal cancer gene of the patient and the known colorectal cancer gene is indicative of a disease state or a propensity for a disease state, as outlined herein.

In a preferred embodiment, the colorectal cancer genes are used as probes to determine the number of copies of the colorectal cancer gene in the genome.

In another preferred embodiment colorectal cancer genes are used as probed to determine the chromosomal localization of the colorectal cancer genes. Information such as chromosomal localization finds use in providing a diagnosis or prognosis in particular when chromosomal abnormalities such as translocations, and the like are identified in colorectal cancer gene loci.

Thus, in one embodiment, methods of modulating colorectal cancer in cells or organisms are provided. In one embodiment, the methods comprise administering to a cell an anti-colorectal cancer antibody that reduces or eliminates the biological activity of an endogeneous colorectal cancer protein. Alternatively, the methods comprise administering to a cell or organism a recombinant nucleic acid encoding a colorectal cancer protein. As will be appreciated by those in the art, this may be accomplished in any number of ways. In a preferred embodiment, for example when the colorectal cancer sequence is down-regulated in colorectal cancer, the activity of the colorectal cancer gene is increased by increasing the amount of colorectal cancer in the cell, for example by overexpressing the endogeneous colorectal cancer or by administering a gene encoding the colorectal cancer sequence, using known gene-therapy techniques, for example. In a preferred embodiment, the gene therapy techniques include the incorporation of the erogenous gene using enhanced homologous recombination (EHR), for example as described in PCT/US93/03868, hereby incorporated by reference in its entirety. Alternatively, for example when the colorectal cancer sequence is up-regulated in colorectal cancer , the activity of the endogeneous colorectal cancer gene is decreased, for example by the administration of a colorectal cancer antisense nucleic acid.

In one embodiment, the colorectal cancer proteins of the present invention may be used to generate polyclonal and monoclonal antibodies to colorectal cancer proteins, which are useful as described herein. Similarly, the colorectal cancer proteins can be coupled, using standard technology, to affinity chromatography columns. These columns may then be used to purify colorectal cancer antibodies. In a preferred embodiment, the antibodies are generated to epitopes unique to a colorectal cancer protein; that is, the antibodies show little or no cross-reactivity to other proteins. These antibodies find use in a number of applications. For example, the colorectal cancer antibodies may be coupled to standard affinity chromatography columns and used to purify colorectal cancer proteins. The antibodies may also be used as blocking polypeptides, as outlined above, since they will specifically bind to the colorectal cancer protein.

In one embodiment, a therapeutically effective dose of a colorectal cancer or modulator thereof is administered to a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for colorectal cancer degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

The administration of the colorectal cancer proteins and modulators of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the colorectal cancer proteins and modulators may be directly applied as a solution or spray.

The pharmaceutical compositions of the present invention comprise a colorectal cancer protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

In a preferred embodiment, colorectal cancer proteins and modulators are administered as therapeutic agents, and can be formulated as outlined above. Similarly, colorectal cancer genes (including both the full-length sequence, partial sequences, or regulatory sequences of the colorectal cancer coding regions) can be administered in gene therapy applications, as is known in the art. These colorectal cancer genes can include antisense applications, either as gene therapy (i.e. for incorporation into the genome) or as antisense compositions, as will be appreciated by those in the art.

In a preferred embodiment, colorectal cancer genes are administered as DNA vaccines, either single genes or combinations of colorectal cancer genes. Naked DNA vaccines are generally known in the art. Brower, Nature Biotechnology, 16:1304-1305 (1998).

In one embodiment, colorectal cancer genes of the present invention are used as DNA vaccines. Methods for the use of genes as DNA vaccines are well known to one of ordinary skill in the art, and include placing a colorectal cancer gene or portion of a colorectal cancer gene under the control of a promoter for expression in a colorectal cancer patient. The colorectal cancer gene used for DNA vaccines can encode full-length colorectal cancer proteins, but more preferably encodes portions of the colorectal cancer proteins including peptides derived from the colorectal cancer protein. In a preferred embodiment a patient is immunized with a DNA vaccine comprising a plurality of nucleotide sequences derived from a colorectal cancer gene. Similarly, it is possible to immunize a patient with a plurality of colorectal cancer genes or portions thereof as defined herein. Without being bound by theory, expression of the polypeptide encoded by the DNA vaccine, cytotoxic T-cells, helper T-cells and antibodies are induced which recognize and destroy or eliminate cells expressing colorectal cancer proteins.

In a preferred embodiment, the DNA vaccines include a gene encoding an adjuvant molecule with the DNA vaccine. Such adjuvant molecules include cytokines that increase the immunogenic response to the colorectal cancer polypeptide encoded by the DNA vaccine. Additional or alternative adjuvants are known to those of ordinary skill in the art and find use in the invention.

In another preferred embodiment colorectal cancer genes find use in generating animal models of colorectal cancer. As is appreciated by one of ordinary skill in the art, when the colorectal cancer gene identified is repressed or diminished in colorectal cancer tissue, gene therapy technology wherein antisense RNA directed to the colorectal cancer gene will also diminish or repress expression of the gene. An animal generated as such serves as an animal model of colorectal cancer that finds use in screening bioactive drug candidates. Similarly, gene knockout technology, for example as a result of homologous recombination with an appropriate gene targeting vector, will result in the absence of the colorectal cancer protein. When desired, tissue-specific expression or knockout of the colorectal cancer protein may be necessary.

It is also possible that the colorectal cancer protein is overexpressed in colorectal cancer. As such, transgenic animals can be generated that overexpress the colorectal cancer protein. Depending on the desired expression level, promoters of various strengths can be employed to express the transgene. Also, the number of copies of the integrated transgene can be determined and compared for a determination of the expression level of the transgene. Animals generated by such methods find use as animal models of colorectal cancer and are additionally useful in screening for bioactive molecules to treat colorectal cancer.

### EXAMPLES

It is understood that the examples described herein in no way serve to limit the true scope of this invention, but rather are presented for illustrative purposes. All references and sequences of accession numbers cited herein are incorporated by reference in their entirety.

### Example 1

### Tissue Preparation, Labeling Chips, and Fingerprints

### Purify Total RNA From Tissue Using TRIzol Reagent

Estimate tissue weight. Homogenize tissue samples in 1 ml of TRIzol per 50 mg of tissue using a Polytron 3100 homogenizer. The generator/probe used depends upon the tissue size. A generator that is too large for the amount of tissue to be homogenized will cause a loss of sample and lower RNA yield. Use the 20 mm generator for tissue weighing more than 0.6 g. If the working volume is greater than 2 ml, then homogenize tissue in a 15 ml polypropylene tube (Falcon 2059). Fill tube no greater than 10 ml.

### Homogenization

Before using generator, it should have been cleaned after last usage by running it through soapy H20 and rinsing thoroughly. Run through with EtOH to sterilize. Keep tissue frozen until ready. Add TRIzol directly to frozen tissue then homogenize.

Following homogenization, remove insoluble material from the homogenate by centrifugation at 7500×g for 15 min. in a Sorvall superspeed or 12,000×g for 10 min. in an Eppendorf centrifuge at 4° C. Transfer the cleared homogenate to a new tube(s). The samples may be frozen now at −60 to −70° C. (and kept for at least one month) or you may continue with the purification.

### Phase Separation

Incubate the homogenized samples for 5 minutes at room temperature.

Add 0.2 ml of chloroform per 1 ml of TRIzol reagent used in the original homogenization.

Cap tubes securely and shake tubes vigorously by hand (do not vortex) for 15 seconds.

Incubate samples at room temp. for 2-3 minutes. Centrifuge samples at 6500 rpm in a Sorvall superspeed for 30 min. at 4° C. (You may spin at up to 12,000×g for 10 min. but you risk breaking your tubes in the centrifuge.)

### RNA Precipitation

Transfer the aqueous phase to a fresh tube. Save the organic phase if isolation of DNA or protein is desired. Add 0.5 ml of isopropyl alcohol per 1 ml of TRIzol reagent used in the original homogenization. Cap tubes securely and invert to mix. Incubate samples at room temp. for 10 minutes. Centrifuge samples at 6500 rpm in Sorvall for 20 min. at 4° C.

### RNA Wash

Pour off the supernate. Wash pellet with cold 75% ethanol. Use 1 ml of 75% ethanol per 1 ml of TRIzol reagent used in the initial homogenization. Cap tubes securely and invert several times to loosen pellet. (Do not vortex). Centrifuge at <8000 rpm (<7500×g) for 5 minutes at 4° C.

Pour off the wash. Carefully transfer pellet to an eppendorf tube (let it slide down the tube into the new tube and use a pipet tip to help guide it in if necessary). Depending on the volumes you are working with, you can decide what size tube(s) you want to precipitate the RNA in. When I tried leaving the RNA in the large 15 ml tube, it took so long to dry (i.e. it did not dry) that I eventually had to transfer it to a smaller tube. Let pellet dry in hood. Resuspend RNA in an appropriate volume of DEPC H20. Try for 2-5 ug/ul. Take absorbance readings.

Purify poly A+mRNA from total RNA or clean up total RNA with Qiagen's RNeasy kit

Purification of poly A+mRNA from total RNA. Heat oligotex suspension to 37° C. and mix immediately before adding to RNA. Incubate Elution Buffer at 7° C. Warm up 2×Binding Buffer at 65° C. if there is precipitate in the buffer. Mix total RNA with DEPC-treated water, 2×Binding Buffer, and Oligotex according to Table 2 on page 16 of the Oligotex Handbook. Incubate for 3 minutes at 65 oC. Incubate for 10 minutes at room temperature.

Centrifuge for 2 minutes at 14,000 to 18,000 g. If centrifuge has a soft setting,” then use it. Remove supernatant without disturbing Oligotex pellet. A little bit of solution can be left behind to reduce the loss of Oligotex. Save sup until certain that satisfactory binding and elution of poly A+mRNA has occurred.

Gently resuspend in Wash Buffer OW2 and pipet onto spin column. Centrifuge the spin column at full speed (soft setting if possible) for 1 minute.

Transfer spin column to a new collection tube and gently resuspend in Wash Buffer OW2 and centrifuge as describe herein.

Transfer spin column to a new tube and elute with 20 to 100 ul of preheated (70° C.) Elution Buffer. Gently resuspend Oligotex resin by pipetting up and down. Centrifuge as above. Repeat elution with fresh elution buffer or use first eluate to keep the elution volume low.

Read absorbance, using diluted Elution Buffer as the blank.

Before proceeding with cDNA synthesis, the mRNA must be precipitated. Some component leftover or in the Elution Buffer from the Oligotex purification procedure will inhibit downstream enzymatic reactions of the mRNA.

### Ethanol Precipitation

Add 0.4 vol. of 7.5 M NH4OAc+2.5 vol. of cold 100% ethanol. Precipitate at −20° C. 1 hour to overnight (or 20-30 min. at −70° C.). Centrifuge at 14,000-16,000×g for 30 minutes at 4° C. Wash pellet with 0.5 ml of 80%ethanol (−20° C.) then centrifuge at 14,000-16,000 ×g for 5 minutes at room temperature. Repeat 80% ethanol wash. Dry the last bit of ethanol from the pellet in the hood. (Do not speed vacuum). Suspend pellet in DEPC H20 at 1 ug/ul concentration.

### Clean up Total RNA using Qiagen's RNeasy Kit

Add no more than 100 ug to an RNeasy column. Adjust sample to a volume of 100 ul with RNase-free water. Add 350 ul Buffer RLT then 250 ul ethanol (100%) to the sample. Mix by pipetting (do not centrifuge) then apply sample to an RNeasy mini spin column. Centrifuge for 15 sec at >10,000 rpm. If concerned about yield, re-apply flowthrough to column and centrifuge again.

Transfer column to a new 2-ml collection tube. Add 500 ul Buffer RPE and centrifuge for 15 sec at >10,000 rpm. Discard flowthrough. Add 500 ul Buffer RPE and centrifuge for 15 sec at >10,000 rpm. Discard flowthrough then centrifuge for 2 min at maximum speed to dry column membrane. Transfer column to a new 1.5-ml collection tube and apply 30-50 ul of RNase-free water directly onto column membrane. Centrifuge 1 min at >10,000 rpm. Repeat elution.

Take absorbance reading. If necessary, ethanol precipitate with ammonium acetate and 2.5×volume 100% ethanol.

Make cDNA using Gibco's “SuperScript Choice System for cDNA Synthesis” kit

### First Strand cDNA Synthesis

Use 5 ug of total RNA or lug of polyA+mRNA as starting material. For total RNA, use 2 ul of SuperScript RT. For polyA+mRNA, use 1 ul of SuperScript RT. Final volume of first strand synthesis mix is 20 ul. RNA must be in a volume no greater than 10 ul. Incubate RNA with 1 ul of 100 pmol T7-T24 oligo for 10 min at 70C. On ice, add 7 ul of: 4 ul 5×1st Strand Buffer, 2 ul of 0.1M DTT, and 1 ul of 1 mM dNTP mix. Incubate at 37C. for 2 min then add SuperScript RT

Incubate at 37C. for 1 hour.

Second Strand Synthesis

Place 1st strand reactions on ice.

Add: 91 ul DEPC H2O

30 ul 5×2nd Strand Buffer

3 ul 10 mM dNTP mix

1 ul 10 U/ul E.coli DNA Ligase

4 ul 10 U/ul E.coli DNA Polymerase

1 ul 2U/ul RNase H

Make the above into a mix if there are more than 2 samples. Mix and incubate 2 hours at 16C.

Add 2 ul T4 DNA Polymerase. Incubate 5 min at 16C. Add 10 ul of 0.5M EDTA

Clean up cDNA

Phenol:Chloroform:Isoamyl Alcohol (25:24:1) purification using Phase-Lock gel tubes:

Centrifuge PLG tubes for 30 sec at maximum speed. Transfer cDNA mix to PLG tube. Add equal volume of phenol:chloroform:isamyl alcohol and shake vigorously (do not vortex). Centrifuge 5 minutes at maximum speed. Transfer top aqueous solution to a new tube. Ethanol precipitate: add 7.55×M NH4Oac and 2.5×volume of 100% ethanol. Centrifuge immediately at room temp. for 20 min, maximum speed. Remove sup then wash pellet 2×with cold 80% ethanol. Remove as much ethanol wash as possible then let pellet air dry. Resuspend pellet in 3 ul RNase-free water.

In vitro Transcription (IVT) and labeling with biotin Pipet 1.5 ul of cDNA into a thin-wall PCR tube.

Make NTP labeling mix:

Combine at room temperature: 2 ul T7 10×ATP (75mM) (Ambion)

2 ul T7 10×GTP (75 mM) (Ambion)

1.5 ul T7 10×CTP (75 mM) (Ambion)

1.5 ul T7 10×UTP (75 mM) (Ambion)

3.75 ul 10 mM Bio-16-UTP (Boehringer-Mannheim/Roche or Enzo) 3.75 ul 10 mM Bio-16-CTP (Enzo)

2 ul 10×T7 transcription buffer (Ambion)

2 ul 10×T7 enzyme mix (Ambion)

Final volume of total reaction is 20 ul. Incubate 6 hours at 37C. in a PCR machine.

RNeasy Clean-up of IVT Product

Follow previous instructions for RNeasy columns or refer to Qiagen's RNeasy protocol handbook.

cRNA will most likely need to be ethanol precipitated. Resuspend in a volume compatible with the fragmentation step.

### Fragmentation

15 ug of labeled RNA is usually fragmented. Try to minimize the fragmentation reaction volume; a 10 ul volume is recommended but 20 ul is all right. Do not go higher than 20 ul because the magnesium in the fragmentation buffer contributes to precipitation in the hybridization buffer.

Fragment RNA by incubation at 94 C. for 35 minutes in 1×Fragmentation buffer.

5×Fragmentation buffer:

200 mM Tris-acetate, pH 8.1

500 mM KOAc

150 mM MgOAc

The labeled RNA transcript can be analyzed before and after fragmentation. Samples can be heated to 65C. for 15 minutes and electrophoresed on 1% agarose/TBE gels to get an approximate idea of the transcript size range

### Hybridization

200 ul (10 ug cRNA) of a hybridization mix is put on the chip. If multiple hybridizations are to be done (such as cycling through a 5 chip set), then it is recommended that an initial hybridization mix of 300 ul or more be made.

Hybrization Mix: fragment labeled RNA (50 ng/ul final conc.)

50 pM 948-b control oligo

1.5 pM BioB

5 pM BioC

25 pM BioD

100 pM CRE

0.1 mg/ml herring sperm DNA

0.5 mg/ml acetylated BSA

to 300 ul with 1×MES hyb. buffer

The instruction manuals for the products used herein are incorporated herein in their entirety.

Labeling Protocol Provided Herein

Hybridization reaction:

Start with non-biotinylated IVT (purified by RNeasy columns)

(see example 1 for steps from tissue to IVT)

| | |
|---|---|
| IVT antisense RNA; 4 μg: | μl |
| Random Hexamers (1 μg/μl): | 4 μl |
| H2O: | μl |
| | 14 μl |

Incubate 70° C., 10 min. Put on ice.

| Reverse transcription: | |
|---|---|
| 5X First Strand (BRL) buffer: | 6 μl |
| 0.1 M DTT: | 3 μl |
| 50X dNTP mix: | 0.6 μl |
| H2O: | 2.4 μl |
| Cy3 or Cy5 dUTP (1 mM): | 3 μl |
| SS RT II (BRL): | 1 μl |
| | 16 μl |

Add to hybridization reaction.

Incubate 30 min., 42° C.

Add 1 μl SSII and let go for another hour.

Put on ice.

50×dNTP mix (25 mM of cold dATP, dCTP, and dGTP, 10 mM of dTTP: 25 μl each of 100 mM dATP, dCTP, and dGTP; 10 μl of 100 mM dTTP to 15 μl H2O. dNTPs from Pharmacia)

RNA Degradation:

86 μl H2O

Add 1.5 μl 1 M NaOH/2 mM EDTA, incubate at 65° C., 10 min.

10 μl 10N NaOH

4 μl 50 mM EDTA

U-Con 30

500 82 l TE/sample spin at 7000 g for 10 min, save flow through for purification

Qiagen Purification:

suspend u-con recovered material in 500 μl buffer PB

proceed w/normal Qiagen protocol

DNAse digest:

Add 1 μl of 1/100 dil of DNAse/30 μl Rx and incubate at 37° C. for 15 min.

5 min 95° C. to denature enzyme

Sample Preparation:

Add:

Cot-1 DNA: 10 μl

50×dNTPs: 1 μl

Na pyro phosphate: 7.5 μl

10 mg/ml Herring sperm DNA 1 ul of 1/10 dilution

21.8 final vol.

Dry down in speed vac.

Resuspend in 15 μl H20.

Add 0.38 μl 10% SDS.

Heat 95° C., 2 min.

Slow cool at room temp. for 20 min.

Put on slide and hybridize overnight at 64° C.

Washing after the Hybridization:

3×SSC/0.03% SDS: 2 min. 37.5 ml 20×SSC+0.75 ml 10% SDS in 250 ml H20

1×SSC: 5 min. 12.5 ml 20×SSC in 250 ml H2O

0.2×SSC: 5 min. 2.5 ml 20×SSC in 250 ml H2O

Dry slides in centrifuge, 1000 RPM, 1 min.

Scan using appropriate Photomultiplier tube (PMT) and fluorescent excitation and emission channels.

The results are shown in Table 1 and Table 2. The lists of genes come from colorectal tumors from a variety of stages of the disease. The genes that are up regulated in the tumors (overall) were also found to be expressed at a limited amount or not at all in the body map. The body map consists of at least 28 tissue types, including Adrenal Gland, Bladder, Bone Marrow, Brain, Breast, Cervix, Colon, Diaphragm, Heart, Kidney, Liver, Lung, Lymph Node, Muscle, Pancreas, Prostate, Rectum, Salivary Gland, Skin, Small Intestine, Spinal Cord, Spleen, Stomach, Testis, Thymus, Thyroid Trachea and Uterus. As indicated, some of the Accession numbers include expression sequence tags (ESTs). Thus, in one embodiment herein, genes within an expression profile, also termed expression profile genes, include ESTs and are not necessarily full length.

Table 1 shows Accession numbers for 1747 genes upregulated in colon tumor tissue. The table provides the exemplar accession numbers, Unigene ID numbers, unique Eos codes, descriptions of the genes encoded, and relative amount of expression as compared with expression in other normal body tissue.

TABLE 1
GENES INVOLVED IN COLORECTAL CANCER

| | | | | | Ratio |
|---|---|---|---|---|---|
| | | | | | TumMet/ |
| Pkey | Probeset | Ex Accn | UniG ID | UniGene Title | Body |
| 332264 | EOS32195 | N72849 | Hs.115263 | epiregulin | 17.6 |
| 332716 | EOS32647 | L00058 | Hs.79070 | v-myc avian myelocytomatosis viral oncogene homolog | 15.0 |
| 312845 | EOS12776 | AI911215 | Hs.186555 | ESTs | 14.3 |
| 310257 | EOS10188 | AW389247 | Hs.148826 | ESTs | 11.6 |
| 322567 | EOS22498 | AF155108 | | EST cluster (not in UniGene) | 11.5 |
| 331060 | EOS30991 | N75081 | Hs.21648 | ESTs | 10.3 |
| 322303 | EOS22234 | W07459 | | EST cluster (not in UniGene) | 9.6 |
| 301891 | EOS01822 | AF131855 | Hs.106127 | Homo sapiens clone 25056 mRNA sequence | 9.5 |
| 318524 | EOS18455 | AW291511 | Hs.253687 | ESTs | 8.9 |
| 314001 | EOS13932 | AW168495 | Hs.8750 | ESTs | 7.8 |
| 331183 | EOS31114 | T40769 | Hs.8469 | EST | 7.3 |
| 315429 | EOS15360 | AW009951 | Hs.206892 | ESTs | 7.3 |
| 303344 | EOS03275 | AA255977 | Hs.250646 | ESTs; Highly similar to ubiquitin-conjugating enzyme [M. musculus] | 6.7 |
| 313625 | EOS13556 | AW468402 | Hs.254020 | ESTs | 6.7 |
| 307084 | EOS07015 | AI160527 | | EST singleton (not in UniGene) with exon hit | 6.1 |
| 314943 | EOS14874 | AI476797 | Hs.184572 | cell division cycle 2; G1 to S and G2 to M | 6.1 |
| 303753 | EOS03684 | AW503733 | Hs.170315 | ESTs | 5.7 |
| 315593 | EOS15524 | AW198103 | Hs.158154 | ESTs | 5.3 |
| 313604 | EOS13535 | AI745325 | Hs.182286 | ESTs; Moderately similar to !!!! ALU SUBFAMILY SB2 WARNING | 5.1 |
| | | | | ENTRY !!!! [H.sapiens] | |
| 312319 | EOS12250 | AA216698 | Hs.180780 | Homo sapiens agnn precursor mRNA; partial cds | 5.1 |
| 312614 | EOS12545 | AI766732 | Hs.201194 | ESTs | 4.8 |
| 323176 | EOS23107 | AW071648 | Hs.123199 | ESTs | 4.8 |
| 317916 | EOS17847 | AI565071 | Hs.159983 | ESTs | 4.7 |
| 301846 | EOS01777 | R20002 | Hs.6823 | ESTs; Weakly similar to intrinsic factor-B12 receptor precursor [H. sapiens] | 4.6 |
| 311157 | EOS11088 | AI990122 | Hs.196988 | ESTs | 4.6 |
| 332640 | EOS32571 | AA417152 | Hs.5101 | protein regulator of cytokinesis 1 | 4.6 |
| 311728 | EOS11659 | AW083000 | Hs.184776 | ribosomal protein L23a | 4.5 |
| 313774 | EOS13705 | AW136836 | Hs.144583 | ESTs | 4.5 |
| 312339 | EOS12270 | AA524394 | | EST cluster (not in UniGene) | 4.4 |
| 315369 | EOS15300 | AA764918 | Hs.256531 | ESTs | 4.3 |
| 303756 | EOS03687 | AI738488 | Hs.115838 | ESTs | 4.3 |
| 301050 | EOS00981 | AW136973 | Hs.144475 | ESTs; Weakly similar to mitogen inducible gene mig-2 [H. sapiens] | 4.3 |
| 300319 | EOS00250 | AW157646 | Hs.153506 | ESTs; Weakly similar to microtubule-actin crosslinking factor [M. musculus] | 4.3 |
| 300664 | EOS00595 | AI444628 | Hs.256809 | ESTs | 4.3 |
| 302655 | EOS02586 | AJ227892 | | EST cluster (not in UniGene) with exon hit | 4.1 |
| 315175 | EOS15106 | AI025842 | Hs.152530 | ESTs | 4.1 |
| 330786 | EOS30717 | D60374 | Hs.258712 | EST | 4.1 |
| 310875 | EOS10806 | T47764 | Hs.132917 | ESTs | 4.1 |
| 313425 | EOS13356 | AA745689 | Hs.186838 | ESTs; Weakly similar to similar to zinc finger 5 protein from Gallus gallus; | 4.0 |
| | | | | U51640 [H. sapiens] | |
| 301804 | EOS01735 | AA581004 | | EST cluster (not in UniGene) with exon hit | 4.0 |
| 332203 | EOS32134 | H4938 | Hs.102082 | EST | 3.9 |
| 322968 | EOS22899 | AI905228 | | EST cluster (not in UniGene) | 3.8 |
| 321524 | EOS21455 | N79126 | | EST cluster (not in UniGene) | 3.8 |
| 302476 | EOS02407 | AF182294 | | EST cluster (not in UniGene) with exon hit | 3.8 |
| 303295 | EOS03226 | AA205625 | Hs.208067 | ESTs | 3.8 |
| 310016 | EOS09947 | AW449612 | Hs.152475 | ESTs | 3.7 |
| 324871 | EOS24802 | AW297755 | Hs.148832 | ESTs | 3.7 |
| 322887 | EOS22818 | AI986306 | Hs.233460 | ESTs; Weakly similar to KIAA0969 protein [H. sapiens] | 3.7 |
| 313171 | EOS13102 | N67879 | Hs.157695 | ESTs | 3.7 |
| 321638 | EOS21569 | AI356352 | Hs.108932 | ESTs | 3.7 |
| 320445 | EOS20376 | R33916 | | EST cluster (not in UniGene) | 3.6 |
| 302149 | EOS02080 | AI383794 | Hs.152337 | protein arginine N-methyltransferase 3(hnRNP methyltransferase S. cerevisiae)-like 3 | 3.6 |
| 316905 | EOS16836 | AW138241 | Hs.210846 | ESTs | 3.6 |
| 313166 | EOS13097 | AI801098 | Hs.151500 | ESTs | 3.6 |
| 323338 | EOS23269 | R74219 | Hs.23348 | S-phase kinase-associated protein 2 (p45) | 3.5 |
| 311434 | EOS11365 | AW016607 | Hs.201582 | ESTs | 3.5 |
| 312742 | EOS12673 | AI650363 | Hs.116462 | ESTs | 3.4 |
| 323587 | EOS23518 | AI905527 | Hs.141901 | ESTs; Moderately similar to !!!! ALU SUBFAMILY SP WARNING | 3.4 |
| | | | | ENTRY !!!! [H. sapiens] | |
| 317390 | EOS17321 | AW136551 | Hs.181245 | ESTs | 3.4 |
| 315282 | EOS15213 | AI222165 | Hs.144923 | ESTs | 3.4 |
| 318565 | EOS18496 | AI440137 | Hs.164989 | ESTs | 3.4 |
| 307586 | EOS07517 | AI285499 | | EST singleton (not in UniGene) with exon hit | 3.4 |
| 321052 | EOS20983 | AW372884 | Hs.240770 | nuclear cap binding protein subunit 2; 20 kD | 3.3 |
| 324338 | EOS24269 | AL138367 | Hs.247514 | ESTs | 3.3 |
| 307517 | EOS07448 | AI275055 | Hs.164989 | ESTs | 3.3 |
| 314852 | EOS14783 | AI903735 | Hs.137527 | ESTs; Weakly similar to X-linked retinopathy protein [H. sapiens] | 3.3 |
| 324657 | EOS24588 | AW451142 | Hs.255628 | ESTs | 3.2 |
| 314912 | EOS14843 | AI431345 | Hs.161784 | ESTs | 3.2 |
| 324790 | EOS24721 | AI334367 | Hs.159337 | ESTs | 3.2 |
| 315498 | EOS15429 | AA628539 | Hs.116252 | ESTs; Moderately similar to !!!! ALU SUBFAMILY J WARNING | 3.2 |
| | | | | ENTRY !!!! [H. sapiens] | |
| 312857 | EOS12788 | AA772279 | Hs.126914 | ESTs | 3.2 |
| 300762 | EOS00693 | AI497778 | Hs.168053 | ESTs | 3.2 |
| 325587 | EOS25518 | c12_hs gi\|6682462\|ref\|gn 1 + 126724 126967 ex 7 7 CDSl 2.44 244 3099 | | | 3.2 |
| | | | | CH.12_hs gi\|6682462 | |
| 320654 | EOS20585 | AW263086 | Hs.118112 | ESTs | 3.2 |
| 316715 | EOS16646 | AI440266 | Hs.170673 | ESTs | 3.1 |
| 333279 | EOS33210 | CH22_522FG_126_1_LINK_EM:AC005500.GENSCAN.8-1 | | | 3.1 |
| | | | | CH22_FGENES.126_1 | |
| 309689 | EOS09620 | AW236171 | Hs.181357 | laminin receptor 1 (67 kD; ribosomal protein SA) | 3.1 |
| 323846 | EOS23777 | AA337621 | Hs.137635 | ESTs | 3.1 |
| 324678 | EOS24609 | AI990739 | Hs.236511 | ESTs; Moderately similar to RNA splicing-related protein [R.norvegicus] | 3.1 |
| 308362 | EOS08293 | AI613519 | | EST singleton (not in UniGene) with exon hit | 3.1 |
| 308615 | EOS08546 | AI738593 | | EST singleton (not in UniGene) with exon hit | 3.0 |
| 315397 | EOS15328 | AA218940 | Hs.137516 | ESTs | 3.0 |
| 302236 | EOS02167 | AI128606 | Hs.167558 | zinc finger protein 161 | 3.0 |
| 321693 | EOS21624 | AA700017 | Hs.173737 | ras-related C3 botulinum toxin substrate 1 (rho family; small GTP binding protein Rac1) | 3.0 |
| 330814 | EOS30745 | AA015730 | Hs.247277 | ESTs; Weakly similar to transformation-related protein [H. sapiens] | 3.0 |
| 302977 | EOS02908 | AW263124 | | EST cluster (not in UniGene) with exon hit | 3.0 |
| 327516 | EOS27447 | c_2_hs gi\|6117815\|ref\|gn 6 + 199078 199216 ex 4 4 CDSl 9.15 139 1551 | | | 2.9 |
| | | | | CH.02_hs gi\|6117815 | |
| 333278 | EOS33209 | CH22_521FG_125_2_LINK_EM:AC005500.GENSCAN.7-2 | | | 2.9 |
| | | | | CH22_FGENES.125_2 | |
| 302088 | EOS02019 | U77629 | Hs.135639 | achaete-scute complex (Drosophila) homolog-like 2 | 2.9 |
| 322718 | EOS22649 | AF150270 | Hs.233322 | ESTs; Weakly similar to cDNA EST EMBL: T01156 comes from this gene [C.elegans] | 2.9 |
| 329154 | EOS29085 | c_x_hs_gi\|5868686\|ref\|gn 2 − 200851 201356 ex 1 3 CDSl 30.28 506 1812 | | | 2.9 |
| | | | | CH.X_hs gi\|5868686 | |
| 315978 | EOS15909 | AA830893 | Hs.119769 | ESTs | 2.9 |
| 302677 | EOS02608 | H63227 | Hs.132880 | ESTs; Highly similar to ubiquitin-conjugating enzyme [M. musculus] | 2.9 |
| 315007 | EOS14938 | AI806583 | Hs.125291 | ESTs | 2.9 |
| 303780 | EOS03711 | AI424014 | Hs.243450 | ESTs; Moderately similar to KIAA0456 protein [H. sapiens] | 2.9 |
| 331362 | EOS31293 | AA417956 | Hs.40782 | ESTs | 2.9 |
| 335815 | EOS35746 | CH22_3187FG_618_3_LINK_EM:AC005500.GENSCAN.510-3 | | | 2.8 |
| | | | | CH22_FGENES.618_3 | |
| 332070 | EOS32001 | AA598545 | Hs.228138 | EST | 2.8 |
| 315720 | EOS15651 | AW291875 | Hs.163900 | ESTs | 2.8 |
| 311913 | EOS11844 | AI358522 | Hs.221417 | ESTs | 2.8 |
| 331014 | EOS30945 | H98597 | Hs.30340 | ESTs | 2.8 |
| 322035 | EOS21966 | AL137517 | | EST cluster (not in UniGene) | 2.8 |
| 338057 | EOS37988 | CH22_6558FG_LINK_EM:AC005500.GENSCAN.160-1 | | | 2.8 |
| | | | | CH22_EM:AC005500.GENSCAN.160-1 | |
| 335829 | EOS35760 | CH22_3202FG_620_3_LINK_EM:AC005500.GENSCAN.512-3 | | | 2.8 |
| | | | | CH22_FGENES.620_3 | |
| 312136 | EOS12067 | AW451469 | Hs.209990 | ESTs | 2.8 |
| 303132 | EOS03063 | AI929819 | Hs.193330 | ESTs | 2.8 |
| 317548 | EOS17479 | AI654187 | Hs.195704 | ESTs | 2.8 |
| 325585 | EOS25516 | c12_hs gi\|6682462\|ref\|gn 1 + 73476 73574 ex 5 7 CDSi 8.52 99 309 | | | 2.7 |
| | | | | CH.12_hs gi\|6682462 | |
| 334631 | EOS34562 | CH22_1939FG_416_7_LINK_EM.AC005500.GENSCAN.277-7 | | | 2.7 |
| | | | | CH22_FGENES.416_7 | |
| 329156 | EOS29087 | c_x_hs gi\|5868686\|ref\|gn 2 − 202013 202341 ex 3 3 CDSf 10.23 329 1814 | | | 2.7 |
| | | | | CH.X_hs gi\|5868686 | |
| 318615 | EOS18546 | AI133617 | Hs.191088 | ESTs | 2.7 |
| 300734 | EOS00665 | AW205197 | Hs.240951 | ESTs | 2.7 |
| 324430 | EOS24361 | AA464018 | | EST cluster (not in UniGene) | 2.7 |
| 322296 | EOS22227 | W76326 | Hs.251937 | ESTs | 2.7 |
| 303842 | EOS03773 | AI337304 | Hs.126268 | ESTs; Weakly similar to similar to PDZ domain [C. elegans] | 2.7 |
| 320909 | EOS20840 | D62269 | | EST cluster (not in UniGene) | 2.7 |
| 325195 | EOS25126 | T20258 | Hs.171443 | ESTs; Weakly similar to actin binding protein MAYVEN [H. sapiens] | 2.7 |
| 324959 | EOS24890 | AW367745 | Hs.143137 | ESTs | 2.7 |
| 309997 | EOS09928 | AI291621 | Hs.145199 | ESTs | 2.7 |
| 329367 | EOS29298 | c_x_hs gi\|5868842\|ref\|gn 1 − 87201 87587 ex 1 4 CDSl 8.13 387 3908 | | | 2.7 |
| | | | | CH.X_hs gi\|5868842 | |
| 316697 | EOS16628 | AW293174 | Hs.252627 | ESTs | 2.7 |
| 313600 | EOS13531 | AA429564 | Hs.185802 | ESTs | 2.7 |
| 301471 | EOS01402 | AA995014 | Hs.129544 | ESTs; Weakly similar to ORF YLL027w [S. cerevisiae] | 2.6 |
| 300810 | EOS00741 | AI076890 | Hs.186949 | ESTs | 2.6 |
| 319976 | EOS19907 | N48809 | Hs.250824 | ESTs | 2.6 |
| 313434 | EOS13365 | W92070 | Hs.231902 | ESTs | 2.6 |
| 333849 | EOS33780 | CH22_1118FG_290_8_LINK_EM:AC005500.GENSCAN.146-7 | | | 2.6 |
| | | | | CH22_FGENES.290_8 | |
| 330744 | EOS30675 | AA406142 | Hs.12393 | dTDP-D-glucose 4;6-dehydratase | 2.6 |
| 309398 | EOS09329 | AW081820 | | EST singleton (not in UniGene) with exon hit | 2.6 |
| 338727 | EOS38658 | CH22_7523FG_LINK_EM:AC005500.GENSCAN.500-2 | | | 2.6 |
| | | | | CH22_EM:AC005500.GENSCAN.500-2 | |
| 324620 | EOS24551 | AA448021 | | EST cluster (not in UniGene) | 2.6 |
| 335755 | EOS35686 | CH22_3122FG_604_4_LINK_EM:AC005500.GENSCAN.493-9 | | | 2.6 |
| | | | | CH22_FGENES.604_4 | |
| 315858 | EOS15789 | AA737345 | | EST cluster (not in UniGene) | 2.6 |
| 307288 | EOS07219 | AI205169 | | EST singleton (not in UniGene) with exon hit | 2.5 |
| 330542 | EOS30473 | U23942 | Hs.226213 | cytochrome P450; 51 (lanosterol 14-alpha-demethylase) | 2.5 |
| 335896 | EOS35827 | CH22_3273FG_635_4_LINK_EM:AC005500.GENSCAN.525-6 | | | 2.5 |
| | | | | CH22_FGENES.635_4 | |
| 316578 | EOS16509 | AA775623 | Hs.211683 | ESTs | 2.5 |
| 329193 | EOS29124 | c_x_hs gi\|5868716\|ref\|gn 3 + 168095 168181 ex 9 9 CDSl-1.11 87 2064 | | | 2.5 |
| | | | | CH.X_hs gi\|5868716 | |
| 315193 | EOS15124 | AI241331 | Hs.131765 | ESTs | 2.5 |
| 319478 | EOS19409 | R06841 | | EST cluster (not in UniGene) | 2.5 |
| 334727 | EOS34658 | CH22_2038FG_424_1_LINK_EM:AC005500.GENSCAN.285-3 | | | 2.5 |
| | | | | CH22_FGENES.424_1 | |
| 328113 | EOS28044 | c_6_hs gi\|5868024\|ref\|gn 2 − 80378 80491 ex 2 3 CDSi 3.89 114 3247 | | | 2.5 |
| | | | | CH.06_hs gi\|5868024 | |
| 315214 | EOS15145 | AI915927 | Hs.34771 | ESTs | 2.5 |
| 324718 | EOS24649 | AI557019 | Hs.116467 | ESTs | 2.5 |
| 313326 | EOS13257 | AI088120 | Hs.122329 | ESTs | 2.5 |
| 319480 | EOS19411 | R06933 | Hs.184221 | ESTs | 2.5 |
| 317902 | EOS17833 | AI828602 | Hs.211265 | ESTs | 2.5 |
| 323341 | EOS23272 | AL134875 | Hs.192386 | ESTs | 2.5 |
| 336003 | EOS35934 | CH22_3385FG_664_4_LINK_DJ32I10.GENSCAN.5-4 | | | 2.5 |
| | | | | CH22_FGENES.664_4 | |
| 322992 | EOS22923 | AA142891 | Hs.193165 | ESTs | 2.5 |
| 314911 | EOS14842 | AW292329 | Hs.163481 | ESTs | 2.5 |
| 313603 | EOS13534 | AW468119 | | EST cluster (not in UniGene) | 2.5 |
| 306469 | EOS06400 | AA983792 | | EST singleton (not in UniGene) with exon hit | 2.5 |
| 324715 | EOS24646 | AI739168 | | EST cluster (not in UniGene) | 2.5 |
| 302455 | EOS02386 | AA356923 | Hs.240770 | nuclear cap binding protein subunit 2; 20 kD | 2.4 |
| 321023 | EOS20954 | H25135 | Hs.125608 | ESTs | 2.4 |
| 302099 | EOS02030 | AL021397 | Hs.137576 | ribosomal protein L34 pseudogene 1 | 2.4 |
| 314092 | EOS14023 | AI984040 | Hs.226946 | ESTs | 2.4 |
| 318587 | EOS18518 | AA779704 | Hs.168830 | ESTs | 2.4 |
| 303702 | EOS03633 | AW500748 | Hs.224961 | ESTs; Weakly similar to 73 kDA subunit of cleavage and polyadenylation specificity | 2.4 |
| | | | | factor [H. sapiens] | |
| 301822 | EOS01753 | X17033 | Hs.1142 | integrin; alpha 2 (CD49B; alpha 2 subunit of VLA-2 receptor) | 2.4 |
| 322694 | EOS22625 | AI110872 | | EST cluster (not in UniGene) | 2.4 |
| 323333 | EOS23264 | AA228883 | | EST cluster (not in UniGene) | 2.4 |
| 301954 | EOS01885 | AJ009936 | Hs.118138 | nuclear receptor subfamily 1; group I; member 2 | 2.4 |
| 331363 | EOS31294 | AA421562 | Hs.91011 | anterior gradient 2 (Xenepus laevis) homolog | 2.4 |
| 303811 | EOS03742 | AW182340 | Hs.246155 | ESTs; Weakly similar to DNA TOPOISOMERASE I [H. sapiens] | 2.4 |
| 308243 | EOS08174 | AI560037 | | EST singleton (not in UniGene) with exon hit | 2.4 |
| 336021 | EOS35952 | CH_22_3404FG_669_10_LINK_DJ32I10.GENSCAN.9-15 | | | 2.4 |
| | | | | CH22_FGENES.669_10 | |
| 334789 | EOS34720 | CH22_2101FG_432_14_LINK_EM:AC005500.GENSCAN.293-17 | | | 2.4 |
| | | | | CH22_FGENES_432_14 | |
| 320807 | EOS20738 | AA086110 | Hs.188536 | Homo sapiens clone 24838 mRNA sequence | 2.4 |
| 328903 | EOS28834 | c_8_hs gi\|5868514\|ref\|gn 1 + 23625 24468 ex 3 5 CDSi 91.18 844 219 | | | 2.4 |
| | | | | CH_08_hs gi\|5868514 | |
| 338759 | EOS38690 | CH22_7581FG_LINK_EM:AC005500.GENSCAN.517-6 | | | 2.3 |
| | | | | CH22_EM:AC005500.GENSCAN.517-6 | |
| 333769 | EOS33700 | CH22_1036FG_271_8_LINK_EM:AC005500.GENSCAN.127-8 | | | 2.3 |
| | | | | CH22_FGENES.271_8 | |
| 303597 | EOS03528 | AI792141 | Hs.143560 | ESTs; Weakly similar to brain mitochondrial carrier protein-1 [H. sapiens] | 2.3 |
| 305898 | EOS05829 | AA872838 | Hs.242463 | keratin 8 | 2.3 |
| 304439 | EOS04370 | AA398882 | | EST singleton (not in UniGene) with exon hit | 2.3 |
| 301604 | EOS01535 | AA373124 | Hs.105837 | ESTs; Weakly similar to C17G10.1 [C.elegans] | 2.3 |
| 315071 | EOS15002 | AA552690 | Hs.152423 | ESTs | 2.3 |
| 330565 | EOS30496 | U51095 | Hs.1545 | caudal type homeo box transcription factor 1 | 2.3 |
| 331589 | EOS31520 | N71027 | Hs.41856 | ESTs | 2.3 |
| 303216 | EOS03147 | AA581439 | Hs.152328 | ESTs | 2.3 |
| 324988 | EOS24919 | T06997 | | EST cluster (not in UniGene) | 2.3 |
| 312996 | EOS12927 | AA249018 | | EST cluster (not in UniGene) | 2.3 |
| 332314 | EOS32245 | T25862 | Hs.101774 | ESTs | 2.3 |
| 313325 | EOS13256 | AI420611 | Hs.127832 | ESTs | 2.3 |
| 322991 | EOS22922 | C18965 | Hs.159473 | ESTs | 2.3 |
| 335496 | EOS35427 | CH22_2848FG_571_4_LINK_EM:AC005500.GENSCAN.460-25 | | | 2.3 |
| | | | | CH22_FGENES.571_4 | |
| 315135 | EOS15066 | AA627561 | Hs.192446 | ESTs | 2.3 |
| 319488 | EOS19419 | AW250340 | | EST cluster (not in UniGene) | 2.3 |
| 323571 | EOS23502 | AA984133 | Hs.153260 | c-Cbl-interacting protein | 2.3 |
| 322826 | EOS22757 | AI807883 | Hs.156932 | ESTs | 2.3 |
| 322221 | EOS22152 | AI890619 | Hs.179662 | nucleosome assembly protein 1-like 1 | 2.3 |
| 312242 | EOS12173 | AI380207 | Hs.125276 | ESTs | 2.3 |
| 315238 | EOS15169 | AA593867 | Hs.170890 | ESTs | 2.3 |
| 315168 | EOS15099 | AA622130 | Hs.152524 | ESTs | 2.3 |
| 300504 | EOS00435 | AW204624 | Hs.192927 | ESTs; Weakly similar to Lim kinase [H. sapiens] | 2.3 |
| 323243 | EOS23174 | W44372 | | EST cluster (not in UniGene) | 2.3 |
| 331628 | EOS31559 | R80965 | Hs.204079 | ESTs | 2.3 |
| 320746 | EOS20677 | AA128302 | | EST cluster (not in UniGene) | 2.3 |
| 324598 | EOS24529 | AA502659 | Hs.163986 | ESTs | 2.3 |
| 308667 | EOS08598 | AI758754 | | EST singleton (not in UniGene) with exon hit | 2.2 |
| 302944 | EOS02875 | AA340708 | Hs.256204 | ESTs; Weakly similar to cyclic nucleotide-gated channel beta subunit [R.norvegicus] | 2.2 |
| 316291 | EOS16222 | AW375974 | Hs.156704 | ESTs | 2.2 |
| 315296 | EOS15227 | AA876905 | Hs.125286 | ESTs | 2.2 |
| 334150 | EOS34081 | CH22_1429FG_339_1_LINK_EM:AC005500.GENSCAN.189-1 | | | 2.2 |
| | | | | CH22_FGENES.339_1 | |
| 331380 | EOS31311 | AA453266 | Hs.246131 | ESTs | 2.2 |
| 321795 | EOS21726 | AI796896 | Hs.222446 | ESTs | 2.2 |
| 331493 | EOS31424 | N34357 | Hs.44571 | ESTs | 2.2 |
| 312890 | EOS12821 | AI813654 | Hs.127478 | ESTs | 2.2 |
| 315583 | EOS15514 | AW003622 | Hs.126555 | ESTs | 2.2 |
| 314306 | EOS14237 | AI697901 | Hs.192425 | ESTs | 2.2 |
| 314138 | EOS14069 | AA740616 | | EST cluster (not in UniGene) | 2.2 |
| 302656 | EOS02587 | AW293005 | Hs.220905 | ESTs | 2.2 |
| 313564 | EOS13495 | AA810141 | Hs.192182 | ESTs | 2.2 |
| 332792 | EOS32723 | CH22_8FG_3_2_LINK_C4G1.GENSCAN.3-2 | | | 2.2 |
| | | | | CH22_FGENES.3_2 | |
| 332020 | EOS31951 | AA488895 | Hs.105219 | ESTs | 2.2 |
| 315143 | EOS15074 | AA878324 | Hs.192734 | ESTs | 2.2 |
| 313385 | EOS13316 | AI032087 | Hs.176711 | ESTs | 2.2 |
| 323835 | EOS23766 | AL042005 | | EST cluster (not in UniGene) | 2.2 |
| 314014 | EOS13945 | AW291847 | Hs.121715 | ESTs; Weakly similar to HP protein [H. sapiens] | 2.2 |
| 336016 | EOS35947 | CH22_3399FG_669_5_LINK_DJ32I10.GENSCAN.g-10 | | | 2.2 |
| | | | | CH22_FGENES.669_5 | |
| 323218 | EOS23149 | AF131846 | Hs.13396 | Homo sapiens clone 25028 mRNA sequence | 2.2 |
| 338059 | EOS37990 | CH22_6561FG_LINK_EM:AC005500.GENSCAN.160-4 | | | 2.2 |
| | | | | CH22_EM:AC005500.GENSCAN.160-4 | |
| 302613 | EOS02544 | AA371059 | Hs.251636 | ubiquitin specific protease 3 | 2.2 |
| 304852 | EOS04783 | AA588595 | | EST singleton (not in UniGene) with exon hit | 2.2 |
| 308457 | EOS08388 | AI669859 | | EST singleton (not in UniGene) with exon hit | 2.2 |
| 311736 | EOS11667 | AA765897 | | EST cluster (not in UniGene) | 2.2 |
| 334183 | EOS34114 | CH22_1464FG_350_13_LINK_EM:AC005500.GENSCAN.209-16 | | | 2.2 |
| | | | | CH22_FGENES.350_13 | |
| 315021 | EOS14952 | AA533447 | | EST cluster (not in UniGene) | 2.2 |
| 303013 | EOS02944 | F07898 | Hs.214190 | interleukin enhancer binding factor 1 | 2.2 |
| 315006 | EOS14937 | AI538613 | Hs.135657 | ESTs | 2.2 |
| 337534 | EOS37465 | CH22_5803FG_{—} | | CH22_FGENES.828-3 | 2.2 |
| | | 828_3_{—} | | | |
| 303276 | EOS03207 | AA431599 | Hs.132799 | ESTs | 2.1 |
| 318617 | EOS18548 | AW247252 | Hs.75514 | nucleoside phosphorylase | 2.1 |
| 330760 | EOS30691 | AA448663 | Hs.30469 | ESTs | 2.1 |
| 319545 | EOS19476 | R83716 | Hs.14355 | ESTs | 2.1 |
| 312252 | EOS12183 | AI128388 | Hs.143655 | ESTs | 2.1 |
| 322882 | EOS22813 | AW248508 | Hs.2491 | DiGeorge syndrome critical region gene 2 | 2.1 |
| 312684 | EOS12615 | AW294020 | Hs.117721 | ESTs | 2.1 |
| 315782 | EOS15713 | AW515455 | Hs.115558 | ESTs; Weakly similar to !!!! ALU SUBFAMILY J WARNING ENTRY !!!! [H. sapiens] | 2.1 |
| 320076 | EOS20007 | AI653733 | Hs.204079 | ESTs | 2.1 |
| 300566 | EOS00497 | H86709 | Hs.21371 | son of sevenless (Drosophila) homolog 1 | 2.1 |
| 300908 | EOS00839 | AA618335 | Hs.146137 | ESTs; Weakly similar to putative [C.elegans] | 2.1 |
| 314778 | EOS14709 | AW079559 | Hs.152258 | ESTs | 2.1 |
| 319233 | EOS19164 | R21054 | Hs.211522 | ESTs | 2.1 |
| 335488 | EOS35419 | CH22_2840FG_570_20_LINK_EM:AC005500.GENSCAN.460-15 | | | 2.1 |
| | | | | CH22_FGENES.570_20 | |
| 334616 | EOS34547 | CH22_1923FG_411_15_LINK_EM:AC005500.GENSCAN.274-22 | | | 2.1 |
| | | | | CH22_FGENES.411_15 | |
| 306792 | EOS06723 | AI042426 | | EST singleton (not in UniGene) with exon hit | 2.1 |
| 301661 | EOS01592 | AI815558 | | EST cluster (not in UniGene) with exon hit | 2.1 |
| 311332 | EOS11263 | AW292247 | Hs.255052 | ESTs | 2.1 |
| 314785 | EOS14716 | AI538226 | Hs.135184 | ESTs | 2.1 |
| 301460 | EOS01391 | AW196758 | Hs.165998 | DKFZP564M2423 protein | 2.1 |
| 332015 | EOS31946 | AA487910 | Hs.208800 | ESTs; Weakly similar to !!!! ALU CLASS B WARNING ENTRY !!!! [H. sapiens] | 2.1 |
| 321529 | EOS21460 | AI269506 | Hs.146066 | ESTs | 2.1 |
| 323740 | EOS23671 | AA324643 | Hs.246106 | ESTs | 2.1 |
| 336019 | EOS35950 | CH22_3402FG_669_8_LINK_DJ32I10.GENSCAN.9-13 | | | 2.1 |
| | | | | CH22_FGENES.669_8 | |
| 314954 | EOS14885 | AA521381 | Hs.187726 | ESTs | 2.1 |
| 303037 | EOS02968 | AF118395 | | EST cluster (not in UniGene) with exon hit | 2.1 |
| 302056 | EOS01987 | AI457532 | Hs.126082 | ESTs; Moderately similar to ROSA26AS [M. musculus] | 2.1 |
| 315178 | EOS15109 | AW362945 | Hs.162459 | ESTs | 2.1 |
| 332246 | EOS32177 | N57927 | Hs.120777 | ESTs; Weakly similar to RNA POLYMERASE II ELONGATION FACTOR | 2.0 |
| | | | | ELL2 [H. sapiens] | |
| 334288 | EOS34219 | CH22_1577FG_369_18_LINK_EM:AC005500.GENSCAN.229-18 | | | 2.0 |
| | | | | CH22_FGENES.369_18 | |
| 324690 | EOS24621 | N88286 | Hs.132808 | ESTs; Weakly similar to Similar to S.pombe-rad4+/cut5+product [H. sapiens] | 2.0 |
| 305257 | EOS05188 | AA679005 | | EST singleton (not in UniGene) with exon hit | 2.0 |
| 311315 | EOS11246 | AW450536 | Hs.209260 | ESTs | 2.0 |
| 311988 | EOS11919 | AW016096 | Hs.13801 | ESTs | 2.0 |
| 302638 | EOS02569 | AA463798 | Hs.102696 | ESTs; Weakly similar to C11D2.4 [C.elegans] | 2.0 |
| 320531 | EOS20462 | W03691 | Hs.24884 | ESTs; Moderately similar to RNA polymerase I associated factor [M. musculus] | 2.0 |
| 323604 | EOS23535 | AI751438 | Hs.182827 | ESTs; Weakly similar to !!!! ALU SUBFAMILY SQ WARNING ENTRY !!!! [H. sapiens] | 2.0 |
| 308852 | EOS08783 | AI829848 | Hs.182937 | peptidylprolyl isomerase A (cyclophilin A) | 2.0 |
| 320521 | EOS20452 | N31464 | Hs.24743 | ESTs | 2.0 |
| 331306 | EOS31237 | AA252079 | Hs.63931 | dachshund (Drosophila) homolog | 2.0 |
| 314941 | EOS14872 | AA515902 | Hs.130650 | ESTs | 2.0 |
| 336684 | EOS36615 | CH22_4167FG_46_1_{—} | | CH22_FGENES.46-1 | 2.0 |
| 301137 | EOS01068 | AF049569 | Hs.137096 | ESTs | 2.0 |
| 338454 | EOS38385 | CH22_7128FG_LINK_EM:AC005500.GENSCAN.360-4 | | | 2.0 |
| | | | | CH22_EM:AC005500.GENSCAN.360-4 | |
| 309700 | EOS09631 | AW241170 | Hs.179661 | Homo sapiens clone 24703 beta-tubulin mRNA; complete cds | 2.0 |
| 330262 | EOS30193 | c_5_p2 gi\|6671884\|gb\|A gn 1 + 67913 68053 ex 3 3 CDSl 5.41 141 597 | | | 2.0 |
| | | | | CH.05_p2 gi\|6671884 | |
| 324163 | EOS24094 | AL046827 | Hs.134651 | ESTs | 2.0 |
| 316493 | EOS16424 | AA766142 | Hs.131810 | ESTs; Weakly similar to !!!! ALU SUBFAMILY J WARNING ENTRY !!!! [H. sapiens] | 2.0 |
| 311873 | EOS11804 | AA730045 | Hs.187866 | ESTs | 2.0 |
| 326757 | EOS26688 | c20_hs gi\|6249610\|ref\|gn 3 + 74531 74597 ex 1 3 CDSf 9.52 67 1416 | | | 2.0 |
| | | | | CH.20_hs gi\|6249610 | |
| 319167 | EOS19098 | F05984 | Hs.250138 | protein phosphatase 2C; magnesium-dependent; catalytic subunit | 2.0 |
| 316011 | EOS15942 | AW516953 | Hs.201372 | ESTs | 2.0 |
| 313635 | EOS13566 | AA507227 | Hs.6390 | ESTs | 2.0 |
| 310027 | EOS09958 | AW449009 | Hs.126647 | ESTs | 2.0 |
| 336662 | EOS36593 | CH22_4138FG_41_1 | | CH22_FGENES.41-1 | 2.0 |
| 334648 | EOS34579 | CH22_1956FG_417_15_LINK_EM:AC005500.GENSCAN.278-15 | | | 2.0 |
| | | | | CH22_FGENES.417_15 | |
| 308676 | EOS08607 | AI761036 | | EST singleton (not in UniGene) with exon hit | 2.0 |
| 312047 | EOS11978 | AA588275 | Hs.14258 | ESTs | 2.0 |
| 324826 | EOS24757 | AA704806 | Hs.143842 | ESTs | 2.0 |
| 322889 | EOS22820 | AA081924 | Hs.211417 | ESTs | 2.0 |
| 316345 | EOS16276 | AW139408 | Hs.152940 | ESTs | 2.0 |
| 313922 | EOS13853 | AI702038 | Hs.100057 | ESTs | 2.0 |
| 319423 | EOS19354 | T83024 | Hs.15119 | ESTs | 2.0 |
| 320244 | EOS20175 | AA296922 | Hs.129778 | gastrointestinal peptide | 2.0 |
| 308957 | EOS08888 | AI869642 | | EST singleton (not in UniGene) with exon hit | 2.0 |
| 334223 | EOS34154 | CH22_1507FG_360_4_LINK_EM:AC005500.GENSCAN.218-4 | | | 1.9 |
| | | | | CH22_FGENES.360_4 | |
| 302980 | EOS02911 | W93435 | | EST cluster (not in UniGene) with exon hit | 1.9 |
| 312153 | EOS12084 | AA759250 | Hs.153028 | cytochrome b-561 | 1.9 |
| 326460 | EOS26391 | c19_hs gi\|5867400\|ref\|gn 3 − 142633 142935 ex 1 2 CDSl 19.03 303 1731 | | | 1.9 |
| | | | | CH.19_hs gi\|5867400 | |
| 319962 | EOS19893 | H06350 | Hs.135056 | ESTs | 1.9 |
| 307064 | EOS06995 | AI149335 | | EST singleton (not in UniGene) with exon hit | 1.9 |
| 331608 | EOS31539 | N89861 | Hs.44162 | ESTs; Weakly similar to cDNA EST yk342h12.5 comes from this gene [C.elegans] | 1.9 |
| 328142 | EOS28073 | c_6_hs_gi\|5868050\|ref\|gn 1 − 9656 9778 ex 2 6 CDSi 11.11 123 3339 | | | 1.9 |
| | | | | CH.06_hs gi\|5868050 | |
| 312527 | EOS12458 | AI695522 | Hs.191271 | ESTs | 1.9 |
| 318581 | EOS18512 | AA769058 | | EST cluster (not in UniGene) | 1.9 |
| 319979 | EOS19910 | AB018281 | Hs.107479 | KIAA0738 gene product | 1.9 |
| 336107 | EOS36038 | CH22_3496FG_696_3_LINK_DA59H18.GENSCAN.4-3 | | | 1.9 |
| | | | | CH22_FGENES.696_3 | |
| 305232 | EOS05163 | AA670052 | Hs.195188 | glyceraldehyde-3-phosphate dehydrogenase | 1.9 |
| 315043 | EOS14974 | AA806538 | Hs.130732 | ESTs | 1.9 |
| 323377 | EOS23308 | AA133260 | Hs.8454 | protein kinase; cAMP-dependent; regulatory; type II; alpha | 1.9 |
| 338260 | EOS38191 | CH22_6863FG_LINK_EM:AC005500.GENSCAN.279-10 | | | 1.9 |
| | | | | CH22_EM:AC005500.GENSCAN.279-10 | |
| 334891 | EOS34822 | CH22_2208FG_452_5_LINK_EM:AC005500.GENSCAN.341-8 | | | 1.9 |
| | | | | CH22_FGENES.452_5 | |
| 316055 | EOS15986 | AA693880 | | EST cluster (not in UniGene) | 1.9 |
| 312414 | EOS12345 | AI915014 | Hs.164235 | ESTs; Weakly similar to !!!! ALU SUBFAMILY J WARNING ENTRY !!!! [H. sapiens] | 1.9 |
| 300225 | EOS00156 | AI989963 | Hs.197505 | ESTs | 1.9 |
| 332607 | EOS32538 | R41791 | Hs.36566 | LIM domain kinase 1 | 1.9 |
| 312405 | EOS12336 | AI523875 | | EST cluster (not in UniGene) | 1.9 |
| 313605 | EOS13536 | AI761786 | Hs.204674 | ESTs | 1.9 |
| 337755 | EOS37686 | CH22_6105FG_LINK_EM:AC000097.GENSCAN.109-2 | | | 1.9 |
| | | | | CH22_EM:AC000097.GENSCAN.109-2 | |
| 323216 | EOS23147 | AA332145 | | EST cluster (not in UniGene) | 1.9 |
| 334872 | EOS34803 | CH22_2186FG_450_2_LINK_EM:AC005500.GENSCAN.339-2 | | | 1.9 |
| | | | | CH22_FGENES.450_2 | |
| 332034 | EOS31965 | AA489847 | Hs.112019 | ESTs; Moderately similar to !!!! ALU SUBFAMILY J WARNING | 1.9 |
| | | | | ENTRY !!!! [H. sapiens] | |
| 332103 | EOS32034 | AA609161 | Hs.112657 | ESTs; Weakly similar to ORF YOR243c [S. cerevisiae] | 1.9 |
| 318196 | EOS18127 | AI056776 | Hs.133397 | ESTs | 1.9 |
| 329141 | EOS29072 | c_x_hs gi\|6017060\|ref\|gn 1 + 343924 343997 ex 2 3 CDSi 8.53 74 1715 | | | 1.9 |
| | | | | CH.X_hs gi\|6017060 | |
| 321539 | EOS21470 | N98619 | Hs.62461 | ARP2 (actin-related protein 2; yeast) homolog | 1.9 |
| 313881 | EOS13812 | AA535580 | Hs.16331 | ESTs | 1.9 |
| 314046 | EOS13977 | AW021917 | Hs.181878 | ESTs | 1.9 |
| 336045 | EOS35976 | CH22_3430FG_679_7_LINK_DJ32I10.GENSCAN.18-8 | | | 1.9 |
| | | | | CH22_FGENES.679_7 | |
| 324799 | EOS24730 | AW272262 | Hs.250468 | ESTs | 1.9 |
| 312656 | EOS12587 | AW152449 | Hs.226469 | ESTs | 1.9 |
| 324662 | EOS24593 | AW504689 | | EST cluster (not in UniGene) | 1.9 |
| 323930 | EOS23861 | AA570698 | Hs.193203 | ESTs | 1.9 |
| 314465 | EOS14396 | AA602917 | Hs.156974 | ESTs | 1.9 |
| 335897 | EOS35828 | CH22_3274FG_635_5_LINK_EM:AC005500.GENSCAN.525-7 | | | 1.9 |
| | | | | CH22_FGENES.635_5 | |
| 321746 | EOS21677 | AI806500 | Hs.102652 | ESTs; Weakly similar to KIAA0437 [H. sapiens] | 1.9 |
| 335687 | EOS35618 | CH22_3048FG_596_2_LINK_EM:AC005500.GENSCAN.488-2 | | | 1.9 |
| | | | | CH22_FGENES.596_2 | |
| 330731 | EOS30662 | AA278816 | Hs.177204 | ESTs | 1.9 |
| 315542 | EOS15473 | AA079476 | Hs.109857 | ESTs; Highly similar to CGI-89 protein [H. sapiens] | 1.9 |
| 336379 | EOS36310 | CH22_3791FG_821_7_LINK_BA232E17.GENSCAN.4-19 | | | 1.9 |
| | | | | CH22_FGENES.821_7 | |
| 305691 | EOS05622 | AA813590 | Hs.119500 | karyopherin alpha 4 (importin alpha 3) | 1.9 |
| 310639 | EOS10570 | AW269082 | Hs.175162 | ESTs | 1.9 |
| 327481 | EOS27412 | c_2_hs gi\|5867783\|ref\|gn 3 + 104472 104673 ex 1 4 CDSf 14.33 202 1308 | | | 1.9 |
| | | | | CH.02_hs gi\|5867783 | |
| 301910 | EOS01841 | T84852 | Hs.98370 | cytochrome P540 family member predicted from ESTs | 1.9 |
| 335478 | EOS35409 | CH22_2830FG_569_1_LINK_EM:AC005500.GENSCAN.456-1 | | | 1.9 |
| | | | | CH22_FGENES.569_1 | |
| 331135 | EOS31066 | R61398 | Hs.4197 | ESTs | 1.9 |
| 335690 | EOS35621 | CH22_3051FG_596_5_LINK_EM:AC005500.GENSCAN.488-5 | | | 1.9 |
| | | | | CH22_FGENES.596_5 | |
| 308047 | EOS07978 | AI459633 | | EST singleton (not in UniGene) with exon hit | 1.9 |
| 334500 | EOS34431 | CH22_1800FG_397_16_LINK_EM:AC005500.GENSCAN.260-18 | | | 1.9 |
| | | | | CH22_FGENES.397_16 | |
| 338250 | EOS38181 | CH22_6848FG_LINK_EM:AC005500.GENSCAN.269-2 | | | 1.8 |
| | | | | CH22_EM:AC005500.GENSCAN.269-2 | |
| 320618 | EOS20549 | AI220276 | Hs.235228 | EST | 1.8 |
| 335044 | EOS34975 | CH22_2367FG_480_1_LINK_EM:AC005500.GENSCAN.374-1 | | | 1.8 |
| | | | | CH22_FGENES.480_1 | |
| 313789 | EOS13720 | AI167810 | Hs.217743 | ESTs | 1.8 |
| 311911 | EOS11842 | AI087123 | Hs.114434 | ESTs; Weakly similar to !!!! ALU SUBFAMILY J WARNING ENTRY !!!! [H. sapiens] | 1.8 |
| 320180 | EOS20111 | AA846203 | Hs.193974 | ESTs; Weakly similar to alternatively spliced product using exon 13A [H. sapiens] | 1.8 |
| 311036 | EOS10967 | AI539227 | Hs.214039 | ESTs | 1.8 |
| 323903 | EOS23834 | AA773580 | Hs.193598 | ESTs | 1.8 |
| 318676 | EOS18607 | T57448 | Hs.15467 | ESTs; Moderately similar to putative phosphoinositide 5-phosphatase type II [M. musculus] | 1.8 |
| 303007 | EOS02938 | AA478876 | Hs.7037 | pallid (mouse) homolog; pallidin | 1.8 |
| 334806 | EOS34737 | CH22_2119FG_435_7_LINK_EM:AC005500.GENSCAN.296-6 | | | 1.8 |
| | | | | CH22_FGENES.435_7 | |
| 311767 | EOS11698 | AI076686 | Hs.190066 | ESTs | 1.8 |
| 331750 | EOS31681 | AA284372 | Hs.111471 | ESTs | 1.8 |
| 314872 | EOS14803 | AI144254 | Hs.239726 | ESTs | 1.8 |
| 314071 | EOS14002 | AA192455 | Hs.188690 | ESTs | 1.8 |
| 328450 | EOS28381 | c_7_hs gi\|5868425\|ref\|gn 2 − 209192 209321 ex 2 3 CDSi 10.41 130 1407 | | | 1.8 |
| | | | | CH.07_hs gi\|5868425 | |
| 328857 | EOS28788 | c_7_hs gi\|6381927\|ref\|gn 3 − 80557 81051 ex 1 1 CDSo 41.51 495 6090 | | | 1.8 |
| | | | | CH.07_hs gi\|6381927 | |
| 313781 | EOS13712 | AA078836 | | EST cluster (not in UniGene) | 1.8 |
| 336953 | EOS36884 | CH22_4746FG_{—} | | CH22_FGENES.361-22 | 1.8 |
| | | 361_22_{—} | | | |
| 300233 | EOS00164 | AI380777 | Hs.189402 | ESTs | 1.8 |
| 326862 | EOS26793 | c20_hs gi\|6552465\|ref\|gn 2 + 107702 107782 ex 12 13 CDSi 3.62 81 2149 | | | 1.8 |
| | | | | CH.20_hs gi\|6552465 | |
| 312364 | EOS12295 | R40111 | Hs.187618 | ESTs | 1.8 |
| 321541 | EOS21472 | AI220292 | Hs.254467 | ESTs | 1.8 |
| 307432 | EOS07363 | AI244259 | Hs.181165 | eukaryotic translation elongation factor 1 alpha 1 | 1.8 |
| 320921 | EOS20852 | R94038 | Hs.199538 | inhibin; beta C | 1.8 |
| 333110 | EOS33041 | CH22_338FG_79_16_LINK_EM:AC000097.GENSCAN.59-15 | | | 1.8 |
| | | | | CH22_FGENES.79_16 | |
| 324914 | EOS24845 | AA847510 | Hs.161292 | ESTs | 1.8 |
| 312681 | EOS12612 | AI028149 | Hs.193124 | pyruvate dehydrogenase kinase; isoenzyme 3 | 1.8 |
| 335697 | EOS35628 | CH22_3058FG_596_12_LINK_EM:AC005500.GENSCAN.488-13 | | | 1.8 |
| | | | | CH22_FGENES.596_12 | |
| 308462 | EOS08393 | AI671311 | | EST singleton (not in UniGene) with exon hit | 1.8 |
| 312138 | EOS12069 | T89405 | Hs.218851 | ESTs; Weakly similar to !!!! ALU SUBFAMILY J WARNING ENTRY !!!! [H. sapiens] | 1.8 |
| 309116 | EOS09047 | AI927149 | Hs.29797 | ribosomal protein L10 | 1.8 |
| 320730 | EOS20661 | AA534539 | Hs.151072 | ESTs | 1.8 |
| 300844 | EOS00775 | AL042759 | Hs.191762 | ESTs | 1.8 |
| 337570 | EOS37501 | CH22_5856FG_LINK_C65E1.GENSCAN.4-2 | | | 1.8 |
| | | | | CH22_C65E1.GENSCAN.4-2 | |
| 332756 | EOS32687 | D63479 | Hs.115907 | diacylglycerol kinase; delta (130 kD) | 1.8 |
| 332161 | EOS32092 | AA621523 | Hs.165464 | ESTs | 1.8 |
| 300942 | EOS00873 | AW275006 | Hs.195969 | ESTs | 1.8 |
| 300680 | EOS00611 | AW468066 | Hs.257712 | ESTs; Weakly similar to KIAA0986 protein [H. sapiens] | 1.8 |
| 328783 | EOS28714 | c_7_hs gi\|5868309\|ref\|gn 5 − 73658 73822 ex 2 5 CDSi 0.78 165 5371 | | | 1.8 |
| | | | | CH.07_hs gi\|5868309 | |
| 307542 | EOS07473 | AI280859 | | EST singleton (not in UniGene) with exon hit | 1.8 |
| 331975 | EOS31906 | AA464972 | Hs.99624 | ESTs | 1.8 |
| 321532 | EOS21463 | T77886 | Hs.83428 | nuclear factor of kappa light polypeptide gene enhancer in B-cells 1 (p105) | 1.8 |
| 318721 | EOS18652 | Z28504 | | EST cluster (not in UniGene) | 1.8 |
| 302124 | EOS02055 | AB023967 | Hs.145078 | regulator of differentiation (in S. pombe) 1 | 1.8 |
| 323541 | EOS23472 | AI185116 | Hs.104613 | ESTs; Weakly similar to Similar to S.cerevisiae hypothetical protein L3111 [H. sapiens] | 1.8 |
| 331057 | EOS30988 | N71399 | Hs.28143 | ESTs | 1.8 |
| 316860 | EOS16791 | AW139099 | Hs.127489 | ESTs | 1.8 |
| 330601 | EOS30532 | U90916 | Hs.82845 | Human clone 23815 mRNA sequence | 1.8 |
| 307334 | EOS07265 | AI214811 | Hs.220615 | ESTs; Weakly similar to TFII-I protein [H. sapiens] | 1.8 |
| 323195 | EOS23126 | AI064982 | Hs.117950 | multifunctional polypeptide similar to SAICAR synthetase and AIR carboxylase | 1.8 |
| 303856 | EOS03787 | AA968589 | Hs.944 | glucose phosphate isomerase | 1.8 |
| 321553 | EOS21484 | H92449 | Hs.116406 | ESTs | 1.8 |
| 332705 | EOS32636 | T59161 | Hs.76293 | thymosin; beta 10 | 1.8 |
| 333139 | EOS33070 | CH22_368FG_83_16_LINK_EM:AC000097.GENSCAN.67-19 | | | 1.8 |
| | | | | CH22_FGENES.83_16 | |
| 338997 | EOS38928 | CH22_7881FG_LINK_DA59H18.GENSCAN.8-22 | | | 1.8 |
| | | | | CH22_DA59H18.GENSCAN.8-22 | |
| 301509 | EOS01440 | AI025435 | Hs.117532 | ESTs | 1.8 |
| 314522 | EOS14453 | AI732301 | Hs.187750 | ESTs; Moderately similar to !!!! ALU CLASS C WARNING ENTRY !!!! [H. sapiens] | 1.8 |
| 303072 | EOS03003 | AF157833 | | EST cluster (not in UniGene) with exon hit | 1.8 |
| 305271 | EOS05202 | AA679895 | | EST singleton (not in UniGene) with exon hit | 1.8 |
| 335287 | EOS35218 | CH22_2629FG_526_11_LINK_EM:AC005500.GENSCAN.420-4 | | | 1.8 |
| | | | | CH22_FGENES.526_11 | |
| 321286 | EOS21217 | AI380940 | | EST cluster (not in UniGene) | 1.8 |
| 318740 | EOS18671 | NM_002543 | | EST cluster (not in UniGene) | 1.8 |
| 323465 | EOS23396 | AA287406 | | EST cluster (not in UniGene) | 1.8 |
| 300611 | EOS00542 | N75450 | | EST cluster (not in UniGene) with exon hit | 1.8 |
| 306235 | EOS06166 | AA932299 | | EST singleton (not in UniGene) with exon hit | 1.8 |
| 336721 | EOS36652 | CH22_4244FG_{—} | | CH22_FGENES.83-17 | 1.8 |
| | | 83_17_{—} | | | |
| 311291 | EOS11222 | AA782601 | Hs.122684 | ESTs | 1.8 |
| 310247 | EOS10178 | AI224982 | Hs.211454 | ESTs | 1.8 |
| 316564 | EOS16495 | AI743571 | Hs.168799 | ESTs; Weakly similar to !!!! ALU SUBFAMILY J WARNING ENTRY !!!! [H. sapiens] | 1.8 |
| 328170 | EOS28101 | c_6_hs gi\|5868071\|ref\|gn 1 + 93170 93295 ex 9 9 CDSl 13.13 126 3591 | | | 1.8 |
| | | | | CH.06_hs gi\|5868071 | |
| 300909 | EOS00840 | AW295479 | Hs.154903 | ESTs; Weakly similar to Abl substrate ena [D. melanogaster] | 1.8 |
| 330869 | EOS30800 | AA115197 | Hs.183702 | ESTs | 1.8 |
| 311048 | EOS10979 | AA506952 | Hs.210508 | ESTs | 1.8 |
| 333764 | EOS33695 | CH22_1031FG_271_3_LINK_EM:AC005500.GENSCAN.127-3 | | | 1.8 |
| | | | | CH22_FGENES.271_3 | |
| 338862 | EOS38793 | CH22_7715FG_LINK_DJ32I10.GENSCAN.1-6 | | | 1.8 |
| | | | | CH22_DJ32I10.GENSCAN.1-6 | |
| 331467 | EOS31398 | N22206 | Hs.43112 | ESTs | 1.8 |
| 327742 | EOS27673 | c_5_hs gi\|5867944\|ref\|gn 3 − 143307 143512 ex 1 3 CDSl 11.07 206 172 | | | 1.8 |
| | | | | CH.05_hs gi\|5867944 | |
| 320955 | EOS20886 | AL049415 | Hs.204290 | Homo sapiens mRNA; cDNA DKFZp586N2119 (from clone DKFZp586N2119) | 1.8 |
| 323589 | EOS23520 | AW390054 | Hs.192843 | ESTs | 1.8 |
| 319951 | EOS19882 | AA307665 | Hs.14559 | ESTs | 1.8 |
| 333763 | EOS33694 | CH22_1030FG_271_2_LINK_EM:AC005500.GENSCAN.127-2 | | | 1.7 |
| | | | | CH22_FGENES.271_2 | |
| 331046 | EOS30977 | N66563 | Hs.191358 | ESTs | 1.7 |
| 320001 | EOS19932 | AA873350 | | EST cluster (not in UniGene) | 1.7 |
| 316869 | EOS16800 | AI954880 | Hs.134604 | ESTs | 1.7 |
| 310774 | EOS10705 | AW134483 | Hs.164371 | ESTs | 1.7 |
| 319379 | EOS19310 | T91443 | Hs.193963 | ESTs | 1.7 |
| 321549 | EOS21480 | AA470984 | Hs.161947 | ESTs | 1.7 |
| 300823 | EOS00754 | AI863068 | Hs.222665 | ESTs; Weakly similar to putative zinc finger protein NY-REN-34 antigen [H. sapiens] | 1.7 |
| 324228 | EOS24159 | AI798146 | Hs.207780 | ESTs | 1.7 |
| 313902 | EOS13833 | AI308165 | Hs.156242 | ESTs | 1.7 |
| 308928 | EOS08859 | AI863908 | | EST singleton (not in UniGene) with exon hit | 1.7 |
| 333770 | EOS33701 | CH22_1037FG_272_1_LINK_EM:AC005500.GENSCAN.127-10 | | | |
| | | | | CH22_FGENES.272_1 | |
| 316934 | EOS16865 | AI571647 | Hs.146170 | ESTs | 1.7 |
| 313219 | EOS13150 | N74924 | Hs.182099 | ESTs | 1.7 |
| 317360 | EOS17291 | AI125252 | Hs.126419 | ESTs | 1.7 |
| 303530 | EOS03461 | AI274851 | Hs.258744 | ESTs | 1.7 |
| 334739 | EOS34670 | CH22_2051FG_424_14_LINK_EM:AC005500.GENSCAN.285-16 | | | 1.7 |
| | | | | CH22_FGENES.424_14 | |
| 337670 | EOS37601 | CH22_5996FG_LINK_EM:AC000097.GENSCAN.57-2 | | | 1.7 |
| | | | | CH22_EM:AC000097.GENSCAN.57-2 | |
| 312079 | EOS12010 | T79745 | Hs.189717 | ESTs | 1.7 |
| 320211 | EOS20142 | AL039402 | Hs.125783 | DEME-6 protein | 1.7 |
| 316218 | EOS16149 | AW207642 | Hs.174021 | ESTs | 1.7 |
| 335682 | EOS35613 | CH22_3043FG_595_2_LINK_EM:AC005500.GENSCAN.487-11 | | | 1.7 |
| | | | | CH22_FGENES.595_2 | |
| 330696 | EOS30627 | AA022632 | Hs.15825 | ESTs | 1.7 |
| 314449 | EOS14380 | AL042667 | Hs.225539 | ESTs | 1.7 |
| 311972 | EOS11903 | N51511 | Hs.188449 | ESTs | 1.7 |
| 307691 | EOS07622 | AI318285 | Hs.182371 | prothymosin; alpha (gene sequence 28) | 1.7 |
| 338249 | EOS38180 | CH22_6847FG_LINK_EM:AC005500.GENSCAN.269-1 | | | 1.7 |
| | | | | CH22_EM:AC005500.GENSCAN.269-1 | |
| 326399 | EOS26330 | c19_hs gi\|5867353\|ref\|gn 1 + 6385 6536 ex 6 6 CDSl 10.69 152 684 | | | 1.7 |
| | | | | CH.19_hs gi\|5867353 | |
| 313290 | EOS13221 | AI753247 | Hs.206454 | ESTs | 1.7 |
| 301615 | EOS01546 | W39477 | | EST cluster (not in UniGene) with exon hit | 1.7 |
| 307034 | EOS06965 | AI142526 | | EST singleton (not in UniGene) with exon hit | 1.7 |
| 313577 | EOS13508 | AA565051 | Hs.155029 | ESTs | 1.7 |
| 324703 | EOS24634 | AB009282 | Hs.31086 | Homo sapiens mRNA for cytochrome b5; partial cds | 1.7 |
| 321317 | EOS21248 | AI937060 | Hs.202040 | ESTs; Weakly similar to KIAA0938 protein [H. sapiens] | 1.7 |
| 312278 | EOS12209 | AW205234 | Hs.201587 | ESTs | 1.7 |
| 333358 | EOS33289 | CH22_604FG_141_9_LINK_EM:AC005500.GENSCAN.21-9 | | | 1.7 |
| | | | | CH22_FGENES.141_9 | |
| 322735 | EOS22666 | AA086123 | | EST cluster (not in UniGene) | 1.7 |
| 326752 | EOS26683 | c20_hs gi\|5867615\|ref\|gn 1 − 1214 1562 ex 2 2 CDSf 33.07 349 1366 | | | 1.7 |
| | | | | CH.20_hs gi\|5867615 | |
| 314733 | EOS14664 | AW452355 | Hs.256037 | ESTs | 1.7 |
| 312902 | EOS12833 | AW292797 | Hs.130316 | ESTs | 1.7 |
| 322653 | EOS22584 | AI828854 | Hs.171891 | ESTs | 1.7 |
| 336015 | EOS35946 | CH22_3398FG_669_4_LINK_DJ32I10.GENSCAN.9-9 | | | 1.7 |
| | | | | CH22_FGENES.669_4 | |
| 324500 | EOS24431 | AW269819 | Hs.169905 | ESTs | 1.7 |
| 310900 | EOS10831 | AI922728 | Hs.165803 | ESTs; Weakly similar to !!!! ALU SUBFAMILY SB WARNING ENTRY !!!! [H. sapiens] | 1.7 |
| 337908 | EOS37839 | CH22_6323FG_LINK_EM:AC005500.GENSCAN.57-1 | | | 1.7 |
| | | | | CH22_EM:AC005500.GENSCAN.57-1 | |
| 304084 | EOS04015 | T91986 | | EST singleton (not in UniGene) with exon hit | 1.7 |
| 332539 | EOS32470 | AA412528 | Hs.20183 | ESTs; Weakly similar to cDNA EST EMBL:T01421 comes from this gene [C.elegans] | 1.7 |
| 314332 | EOS14263 | AL037551 | Hs.95612 | ESTs | 1.7 |
| 321412 | EOS21343 | AW366305 | | EST cluster (not in UniGene) | 1.7 |
| 312187 | EOS12118 | AA700439 | Hs.188490 | ESTs | 1.7 |
| 314147 | EOS14078 | AI656135 | Hs.129805 | ESTs | 1.7 |
| 303131 | EOS03062 | AW081061 | Hs.103180 | actin-like 6 | 1.7 |
| 331341 | EOS31272 | AA303125 | Hs.119009 | ESTs; Weakly similar to !!!! ALU SUBFAMILY SB2 WARNING ENTRY !!!! [H.sapiens] | 1.7 |
| 313615 | EOS13546 | AW295194 | Hs.25264 | DKFZP434N126 protein | 1.7 |
| 329598 | EOS29529 | c10_p2 gi\|3962482\|gb\|A gn 4 + 39924 40220 ex 2 3 CDSi 8.71 297 420 | | | 1.7 |
| | | | | CH.10_p2 gi\|3962482 | |
| 303579 | EOS03510 | AA381124 | Hs.193353 | ESTs; Weakly similar to !!!! ALU SUBFAMILY J WARNING ENTRY !!!! [H. sapiens] | 1.7 |
| 331692 | EOS31623 | W93592 | Hs.47343 | ESTs | 1.7 |
| 323977 | EOS23908 | AW328177 | Hs.234713 | ESTs | 1.7 |
| 332930 | EOS32861 | CH22_151FG_38_4_LINK_C20H12.GENSCAN.29-4 | | | 1.7 |
| | | | | CH22_FGENES.38_4 | |
| 326596 | EOS26527 | c19_hs gi\|6138928\|ref\|gn 4 + 133386 133563 ex 7 9 CDSi - 1.32 178 3520 | | | 1.7 |
| | | | | CH.19_hs gi\|6138928 | |
| 314946 | EOS14877 | AI097229 | Hs.217484 | ESTs; Weakly similar to !!!! ALU SUBFAMILY J WARNING ENTRY !!!! [H. sapiens] | 1.7 |
| 315357 | EOS15288 | AA608684 | Hs.121705 | ESTs; Moderately similar to !!!! ALU CLASS C WARNING ENTRY !!!! [H. sapiens] | 1.7 |
| 324728 | EOS24659 | AA303024 | | EST cluster (not in UniGene) | 1.7 |
| 317501 | EOS17432 | AA931245 | Hs.137097 | ESTs | 1.7 |
| 332219 | EOS32150 | N22508 | Hs.139315 | ESTs | 1.7 |
| 335369 | EOS35300 | CH22_2718FG_543_7_LINK_EM:AC005500.GENSCAN.432-9 | | | 1.7 |
| | | | | CH22_FGENES.543_7 | |
| 322417 | EOS22348 | W36286 | Hs.171873 | ESTs; Weakly similar to PUTATIVE STEROID | 1.7 |
| | | | | DEHYDROGENASE KIK-I [M. musculus] | |
| 316100 | EOS16031 | AW203986 | Hs.213003 | ESTs | 1.7 |
| 314866 | EOS14797 | AW305124 | Hs.191682 | ESTs | 1.7 |
| 300328 | EOS00259 | AW015860 | Hs.224623 | ESTs | 1.7 |
| 315676 | EOS15607 | AW002565 | Hs.136590 | ESTs | 1.7 |
| 314183 | EOS14114 | AA748600 | | EST cluster (not in UniGene) | 1.7 |
| 321354 | EOS21285 | AA078493 | | EST cluster (not in UniGene) | 1.7 |
| 311904 | EOS11835 | T86907 | Hs.119371 | ESTs | 1.7 |
| 322890 | EOS22821 | AA082030 | | EST cluster (not in UniGene) | 1.7 |
| 302759 | EOS02690 | AI885815 | Hs.184727 | ESTs | 1.7 |
| 324600 | EOS24531 | AA503297 | Hs.117108 | ESTs | 1.7 |
| 314973 | EOS14904 | AW273128 | Hs.254669 | EST | 1.7 |
| 324432 | EOS24363 | AA464510 | | EST cluster (not in UniGene) | 1.7 |
| 331520 | EOS31451 | N49068 | Hs.93966 | ESTs | 1.7 |
| 308380 | EOS08311 | AI623988 | | EST singleton (not in UniGene) with exon hit | 1.7 |
| 331010 | EOS30941 | H95039 | Hs.32168 | KIAA0442 protein | 1.7 |
| 325363 | EOS25294 | c12_hs gi\|5866920\|ref\|gn 7 + 700446 700516 ex 6 8 CDSi - 0 6.58 71 113 | | | 1.7 |
| | | | | CH.12_hs gi\|5866920 | |
| 310470 | EOS10401 | AI281848 | Hs.165547 | ESTs | 1.7 |
| 330711 | EOS30642 | AA164687 | Hs.177576 | mannosyl (alpha-1;3-)-glycoprotein beta-1;4-N-acetylglucosaminyltransferase; isoenzyme A | 1.7 |
| 332074 | EOS32005 | AA599012 | Hs.22826 | ESTs | 1.7 |
| 309732 | EOS09663 | AW262211 | Hs.5662 | guanine nucleotide binding protein (G protein); beta polypeptide 2-like 1 | 1.6 |
| 306337 | EOS06268 | AA954221 | Hs.73742 | ribosomal protein; large; P0 | 1.6 |
| 335189 | EOS35120 | CH22_2525FG_507_4_LINK_EM:AC005500.GENSCAN.400-4 | | | 1.6 |
| | | | | CH22_FGENES.507_4 | |
| 316253 | EOS16184 | AI919537 | Hs.118056 | ESTs | 1.6 |
| 332908 | EOS32839 | CH22_129FG_36_12_LINK_C20H12.GENSCAN.28-9 | | | 1.6 |
| | | | | CH22_FGENES.36_12 | |
| 310002 | EOS09933 | AI439096 | Hs.25832 | ESTs | 1.6 |
| 332258 | EOS32189 | N68670 | Hs.103808 | ESTs; Weakly similar to RanBPM [H. sapiens] | 1.6 |
| 336182 | EOS36113 | CH22_3576FG_715_2_LINK_DA59H18.GENSCAN.19-3 | | | 1.6 |
| | | | | CH22_FGENES.715_2 | |
| 328987 | EOS28918 | c_9_hs gi\|5868535\|ref\|gn 1 − 25705 25764 ex 3 10 CDSi 9.90 60 438 | | | 1.6 |
| | | | | CH.09_hs gi\|5868535 | |
| 324481 | EOS24412 | AI916284 | Hs.199671 | ESTs | 1.6 |
| 331406 | EOS31337 | AA610064 | Hs.23440 | KIAA1105 protein | 1.6 |
| 332280 | EOS32211 | R38100 | Hs.106294 | ESTs | 1.6 |
| 332173 | EOS32104 | F09281 | Hs.90424 | ESTs | 1.6 |
| 335739 | EOS35670 | CH22_3102FG_601_10_LINK_EM:AC005500.GENSCAN.491-10 | | | 1.6 |
| | | | | CH22_FGENES.601_10 | |
| 332104 | EOS32035 | AA609177 | Hs.109363 | ESTs | 1.6 |
| 315033 | EOS14964 | AI493046 | Hs.146133 | ESTs | 1.6 |
| 334740 | EOS34671 | CH22_2052FG_424_15_LINK_EM:AC005500.GENSCAN.285-17 | | | 1.6 |
| | | | | CH22_FGENES.424_15 | |
| 334783 | EOS34714 | CH22_2095FG_432_8_LINK_EM:AC005500.GENSCAN.293-11 | | | 1.6 |
| | | | | CH22_FGENES.432_8 | |
| 308010 | EOS07941 | AI439190 | Hs.181165 | eukaryotic translation elongation factor 1 alpha 1 | 1.6 |
| 304521 | EOS04452 | AA464716 | | EST singleton (not in UniGene) with exon hit | 1.6 |
| 318719 | EOS18650 | Z25900 | Hs.18724 | Homo sapiens mRNA; cDNA DKFZp564F093 (from clone DKFZp564F093) | 1.6 |
| 321920 | EOS21851 | N63915 | | EST cluster (not in UniGene) | 1.6 |
| 315019 | EOS14950 | AA532807 | Hs.105822 | ESTs | 1.6 |
| 320793 | EOS20724 | AL049980 | Hs.184216 | DKFZP564C152 protein | 1.6 |
| 305371 | EOS05302 | AA714180 | | EST singleton (not in UniGene) with exon hit | 1.6 |
| 305054 | EOS04985 | AA634127 | Hs.182426 | ribosomal protein S2 | 1.6 |
| 314643 | EOS14574 | AI587502 | Hs.192088 | ESTs | 1.6 |
| 308186 | EOS08117 | AI537940 | | EST singleton (not in UniGene) with exon hit | 1.6 |
| 319371 | EOS19302 | R00321 | Hs.174928 | ESTs | 1.6 |
| 331700 | EOS31631 | Z40011 | Hs.180582 | ESTs | 1.6 |
| 316955 | EOS16886 | AW203959 | Hs.149532 | ESTs | 1.6 |
| 314961 | EOS14892 | AW008061 | Hs.231994 | ESTs | 1.6 |
| 336676 | EOS36607 | CH22_4154FG_43_4_{—} | | CH22_FGENES.43-4 | 1.6 |
| 322801 | EOS22732 | AI831910 | Hs.163734 | ESTs | 1.6 |
| 303363 | EOS03294 | AI964095 | Hs.226801 | ESTs; Weakly similar to DIA-156 protein [H. sapiens] | 1.6 |
| 328105 | EOS28036 | c_6_hs gi\|5868020\|ref\|gn 11 − 301705 301784 ex 4 7 CDSi 5.30 80 3147 | | | 1.6 |
| | | | | CH.06_hs gi\|5868020 | |
| 325481 | EOS25412 | c12_hs gi\|5866957\|ref\|gn 3 + 47590 47672 ex 4 7 CDSi 2.69 83 1895 | | | 1.6 |
| | | | | CH.12_hs gi\|5866957 | |
| 315361 | EOS15292 | AI335229 | Hs.122031 | ESTs | 1.6 |
| 324902 | EOS24833 | D31323 | Hs.211188 | ESTs | 1.6 |
| 336018 | EOS35949 | CH22_3401FG_669_7_LINK_DJ32I10.GENSCAN.9-12 | | | 1.6 |
| | | | | CH22_FGENES.669_7 | |
| 308747 | EOS08678 | AI804500 | Hs.181165 | eukaryotic translation elongation factor 1 alpha 1 | 1.6 |
| 328251 | EOS28182 | c_6_hs gi\|6381891\|ref\|gn 4 + 124444 124557 ex 2 3 CDSi 0.40 114 4554 | | | 1.6 |
| | | | | CH.06_hs gi\|6381891 | |
| 303153 | EOS03084 | U09759 | Hs.8325 | mitogen-activated protein kinase 9 | 1.6 |
| 327809 | EOS27740 | c_5_hs gi\|5867968\|ref\|gn 3 + 54610 54761 ex 4 4 CDSl 0.78 152 993 | | | 1.6 |
| | | | | CH.05_hs gi\|5867968 | |
| 314107 | EOS14038 | AA806113 | Hs.189025 | ESTs | 1.6 |
| 300304 | EOS00235 | AI637934 | Hs.224978 | ESTs | 1.6 |
| 313009 | EOS12940 | W52010 | Hs.191379 | ESTs | 1.6 |
| 331074 | EOS31005 | R08440 | | yf19f9.s1 Soares fetal liver spleen 1NFLS Homo sapiens cDNA clone | 1.6 |
| | | | | IMAGE:127337 3′ similar to | |
| | | | | contains Alu repetitive element;, mRNA sequence | |
| 335773 | EOS35704 | CH22_3142FG_607_9_LINK_EM:AC005500.GENSCAN.496-4 | | | 1.6 |
| | | | | CH22_FGENES.607_9 | |
| 334991 | EOS34922 | CH22_2312FG_469_11_LINK_EM:AC005500.GENSCAN.365-11 | | | 1.6 |
| | | | | CH22_FGENES.469_11 | |
| 322959 | EOS22890 | AI267606 | | EST cluster (not in UniGene) | 1.6 |
| 323731 | EOS23662 | AA323414 | | EST cluster (not in UniGene) | 1.6 |
| 331073 | EOS31004 | R07998 | Hs.18628 | ESTs; Weakly similar to !!!! ALU SUBFAMILY J WARNING ENTRY !!!! [H. sapiens] | 1.6 |
| 313573 | EOS13504 | AI076259 | Hs.190337 | ESTs | 1.6 |
| 316949 | EOS16880 | AA856749 | Hs.124620 | ESTs | 1.6 |
| 328084 | EOS28015 | c_6_hs gi\|6469819\|ref\|gn 3 − 155366 155459 ex 1 4 CDSl 1.23 94 2982 | | | 1.6 |
| | | | | CH.06_hs gi\|6469819 | |
| 331526 | EOS31457 | N49967 | Hs.46624 | ESTs | 1.6 |
| 317987 | EOS17918 | AW138174 | Hs.130651 | ESTs | 1.6 |
| 325594 | EOS25525 | c13_hs gi\|5866992\|ref\|gn 4 − 470474 470566 ex 2 3 CDSi 8.09 93 68 | | | 1.6 |
| | | | | CH.13_hs gi\|5866992 | |
| 310848 | EOS10779 | AI459554 | Hs.161286 | ESTs | 1.6 |
| 309268 | EOS09199 | AI985821 | Hs.62954 | ferritin; heavy polypeptide 1 | 1.6 |
| 304518 | EOS04449 | AA461438 | | EST singleton (not in UniGene) with exon hit | 1.6 |
| 331065 | EOS30996 | N90584 | Hs.9167 | Homo sapiens clone 25085 mRNA sequence | 1.6 |
| 306501 | EOS06432 | AA987294 | | EST singleton (not in UniGene) with exon hit | 1.6 |
| 323289 | EOS23220 | AL134235 | Hs.222442 | ESTs | 1.6 |
| 334630 | EOS34561 | CH22_1938FG_416_6_LINK_EM:AC005500.GENSCAN.277-6 | | | 1.6 |
| | | | | CH22_FGENES.416_6 | |
| 302025 | EOS01956 | AI091466 | Hs.127241 | DKFZP564F052 protein | 1.6 |
| 328998 | EOS28929 | c_9_hs gi\|5868538\|ref\|gn 1 + 40996 41104 ex 1 3 CDSf 11.00 109 480 | | | 1.6 |
| | | | | CH.09_hs gi\|5868538 | |
| 313197 | EOS13128 | AI738851 | Hs.222487 | ESTs | 1.6 |
| 338763 | EOS38694 | CH22_7585FG_LINK_EM:AC005500.GENSCAN.517-16 | | | 1.6 |
| | | | | CH22_EM:AC005500.GENSCAN.517-16 | |
| 332247 | EOS32178 | N58172 | Hs.109370 | ESTs | 1.6 |
| 316724 | EOS16655 | AA810788 | Hs.123337 | ESTs | 1.6 |
| 303306 | EOS03237 | AA215297 | | EST cluster (not in UniGene) with exon hit | 1.6 |
| 306336 | EOS06267 | AA954198 | | EST singleton (not in UniGene) with exon hit | 1.6 |
| 308256 | EOS08187 | AI565498 | | EST singleton (not in UniGene) with exon hit | 1.6 |
| 307056 | EOS06987 | AI148675 | | EST singleton (not in UniGene) with exon hit | 1.6 |
| 321370 | EOS21301 | AJ227900 | | EST cluster (not in UniGene) | 1.6 |
| 336262 | EOS36193 | CH22_3661FG_754_9_LINK_DA59H18.GENSCAN.57-11 | | | 1.6 |
| | | | | CH22_FGENES.754_9 | |
| 335497 | EOS35428 | CH22_2849FG_571_5_LINK_EM:AC005500.GENSCAN.460-26 | | | 1.6 |
| | | | | CH22_FGENES.571_5 | |
| 309582 | EOS09513 | AW169657 | | EST singleton (not in UniGene) with exon hit | 1.6 |
| 329563 | EOS29494 | c10_p2 gi\|3962490\|gb\|A gn 1 − 410 635 ex 2 2 CDSf 13.80 226 267 | | | 1.6 |
| | | | | CH.10_p2 gi\|3962490 | |
| 332504 | EOS32435 | AA053917 | Hs.15106 | chromosome 14 open reading frame 1 | 1.6 |
| 308090 | EOS08021 | AI474601 | Hs.2186 | eukaryotic translation elongation factor 1 gamma | 1.6 |
| 331752 | EOS31683 | AA287312 | Hs.191648 | ESTs | 1.6 |
| 330881 | EOS30812 | AA132986 | Hs.69321 | ESTs; Weakly similar to Similiar to mucin and several other | 1.6 |
| | | | | Ser-Thr-rich proteins [S.cerevisiae] | |
| 315647 | EOS15578 | AA648983 | Hs.212911 | ESTs | 1.6 |
| 336766 | EOS36697 | CH22_4341FG_{—} | | CH22_FGENES.143-20 | 1.6 |
| | | 143_20_{—} | | | |
| 302592 | EOS02523 | AA294921 | Hs.250811 | v-ral simian leukemia viral oncogene homolog B (ras related; GTP binding protein) | 1.6 |
| 315076 | EOS15007 | AI623817 | Hs.168457 | ESTs | 1.6 |
| 337056 | EOS36987 | CH22_4946FG_{—} | | CH22_FGENES.441-4 | 1.6 |
| | | 441_4_{—} | | | |
| 322175 | EOS22106 | AF085975 | | EST cluster (not in UniGene) | 1.6 |
| 336833 | EOS36764 | CH22_4504FG_{—} | | CH22_FGENES.242-2 | 1.6 |
| | | 242_2_{—} | | | |
| 334902 | EOS34833 | CH22_2219FG_452_16_LINK_EM:AC005500.GENSCAN.341-19 | | | 1.6 |
| | | | | CH22_FGENES.452_16 | |
| 318671 | EOS18602 | AA188823 | Hs.212621 | ESTs | 1.6 |
| 308064 | EOS07995 | AI469273 | Hs.181165 | eukaryotic translation elongation factor 1 alpha 1 | 1.6 |
| 320559 | EOS20490 | AB021981 | Hs.159322 | solute carrier family 35 (UDP-N-acetylglucosamine (UDP-GlcNAc) transporter); member 3 | 1.6 |
| 317881 | EOS17812 | AI827248 | Hs.224398 | ESTs | 1.6 |
| 313078 | EOS13009 | N49730 | | EST cluster (not in UniGene) | 1.6 |
| 338689 | EOS38620 | CH22_7464FG_LINK_EM:AC005500.GENSCAN.475-3 | | | 1.6 |
| | | | | CH22_EM:AC005500.GENSCAN.475-3 | |
| 311804 | EOS11735 | AA135159 | Hs.203349 | ESTs | 1.6 |
| 316359 | EOS16290 | AI472213 | Hs.123415 | ESTs | 1.6 |
| 330182 | EOS30113 | c_4_p2 gi\|5123954\|emb\|gn 4 + 120156 120245 ex 2 2 CDSl 4.69 90 11 | | | 1.6 |
| | | | | CH.04_p2 gi\|5123954 | |
| 334718 | EOS34649 | CH22_2028FG_421_29_LINK_EM:AC005500.GENSCAN.282-29 | | | 1.6 |
| | | | | CH22_FGENES.421_29 | |
| 324196 | EOS24127 | AA405524 | Hs.178000 | ESTs | 1.6 |
| 305350 | EOS05281 | AA706676 | | EST singleton (not in UniGene) with exon hit | 1.6 |
| 331469 | EOS31400 | N22273 | Hs.39140 | ESTs | 1.6 |
| 305715 | EOS05646 | AA826884 | | EST singleton (not in UniGene) with exon hit | 1.6 |
| 314460 | EOS14391 | AI263231 | Hs.145607 | ESTs | 1.6 |
| 317634 | EOS17565 | AA953088 | Hs.127550 | ESTs | 1.6 |
| 335293 | EOS35224 | CH22_2635FG_527_6_LINK_EM:AC005500.GENSCAN.421-9 | | | 1.6 |
| | | | | CH22_FGENES.527_6 | |
| 305611 | EOS05542 | AA782331 | | EST singleton (not in UniGene) with exon hit | 1.6 |
| 310430 | EOS10361 | AI670843 | Hs.200257 | ESTs | 1.6 |
| 323696 | EOS23627 | AA641201 | Hs.222051 | ESTs | 1.6 |
| 300610 | EOS00541 | N72596 | Hs.99120 | DEAD/H (Asp-Glu-Ala-Asp/His) box polypeptide; Y chromosome | 1.6 |
| 327364 | EOS27295 | c_1_hs gi\|6552412\|ref\|gn 2 − 115235 115396 ex 1 9 CDSl 2.77 162 3007 | | | 1.6 |
| | | | | CH.01_hs gi\|6552412 | |
| 324848 | EOS24779 | AW021857 | | EST cluster (not in UniGene) | 1.6 |
| 321491 | EOS21422 | H70665 | Hs.183960 | ESTs | 1.6 |
| 336367 | EOS36298 | CH22_3779FG_818_11_LINK_BA232E17.GENSCAN.3-17 | | | 1.6 |
| | | | | CH22_FGENES.818_11 | |
| 331549 | EOS31480 | N56866 | Hs.237507 | EST | 1.6 |
| 328332 | EOS28263 | c_7_hs gi\|5868375\|ref\|gn 6 + 280154 280289 ex 3 5 CDSi-1.04 136 516 | | | 1.5 |
| | | | | CH.07_hs gi\|5868375 | |
| 322817 | EOS22748 | C02420 | | EST cluster (not in UniGene) | 1.5 |
| 303983 | EOS03914 | AW514111 | Hs.181165 | eukaryotic translation elongation factor 1 alpha 1 | 1.5 |
| 329434 | EOS29365 | c_y_hs gi\|5868883\|ref\|gn 1 − 31124 31263 ex 3 20 CDSi 6.38 140 241 | | | 1.5 |
| | | | | CH.Y_hs gi\|5868883 | |
| 338196 | EOS38127 | CH22_6763FG_LINK_EM:AC005500.GENSCAN.235-16 | | | 1.5 |
| | | | | CH22_EM:AC005500.GENSCAN.235-16 | |
| 308488 | EOS08419 | AI682148 | Hs.179661 | Homo sapiens clone 24703 beta-tubulin mRNA; complete cds | 1.5 |
| 314883 | EOS14814 | AW178807 | Hs.246182 | ESTs | 1.5 |
| 307095 | EOS07026 | AI167910 | | EST singleton (not in UniGene) with exon hit | 1.5 |
| 306953 | EOS06884 | AI124971 | | EST singleton (not in UniGene) with exon hit | 1.5 |
| 331786 | EOS31717 | AA398539 | Hs.97369 | EST | 1.5 |
| 303509 | EOS03440 | AW378236 | Hs.256050 | ESTs | 1.5 |
| 324515 | EOS24446 | AW501686 | Hs.163539 | ESTs | 1.5 |
| 339323 | EOS39254 | CH22_8284FG_LINK_BA354I12.GENSCAN.23-2 | | | |
| | CH22_BA354I12.GENSCAN.23-2 | | | 1.5 | |
| 306563 | EOS06494 | AA995296 | | EST singleton (not in UniGene) with exon hit | 1.5 |
| 316076 | EOS16007 | AW297895 | Hs.116424 | ESTs | 1.5 |
| 325622 | EOS25553 | c14_hs gi\|5867000\|ref\|gn 2 + 69994 70075 ex 6 8 CDSi 9.40 82 194 | | | 1.5 |
| | | | | CH.14_hs gi\|5867000 | |
| 309632 | EOS09563 | AW193261 | Hs.156110 | Immunoglobulin kappa variable 1D-8 | 1.5 |
| 314926 | EOS14857 | AI380838 | Hs.124835 | ESTs | 1.5 |
| 314458 | EOS14389 | AI217440 | Hs.143873 | ESTs | 1.5 |
| 335219 | EOS35150 | CH22_2558FG_513_2_LINK_EM:AC005500.GENSCAN.406-2 | | | 1.5 |
| | | | | CH22_FGENES.513_2 | |
| 301079 | EOS01010 | AA305047 | Hs.183654 | ESTs; Weakly similar to unknown [S. cerevisiae] | 1.5 |
| 334122 | EOS34053 | CH22_1400FG_333_3_LINK_EM:AC005500.GENSCAN.185-27 | | | 1.5 |
| | | | | CH22_FGENES.333_3 | |
| 308139 | EOS08070 | AI494477 | | EST singleton (not in UniGene) with exon hit | 1.5 |
| 317412 | EOS17343 | AI301528 | Hs.132604 | ESTs | 1.5 |
| 315073 | EOS15004 | AW452948 | Hs.257631 | ESTs | 1.5 |
| 313139 | EOS13070 | AA362113 | | EST cluster (not in UniGene) | 1.5 |
| 307012 | EOS06943 | AI140212 | | EST singleton (not in UniGene) with exon hit | 1.5 |
| 322895 | EOS22826 | AW470295 | Hs.192152 | ESTs | 1.5 |
| 303779 | EOS03710 | AA897296 | Hs.221266 | ESTs | 1.5 |
| 312344 | EOS12275 | AI742618 | Hs.181733 | ESTs; Weakly similar to nitrilase homolog 1 [H. sapiens] | 1.5 |
| 323632 | EOS23563 | AL039950 | | EST cluster (not in UniGene) | 1.5 |
| 332336 | EOS32267 | T96130 | Hs.137551 | ESTs | 1.5 |
| 304547 | EOS04478 | AA486189 | | EST singleton (not in UniGene) with exon hit | 1.5 |
| 335692 | EOS35623 | CH22_3053FG_596_7_LINK_EM:AC005500.GENSCAN.488-7 | | | 1.5 |
| | | | | CH22_FGENES.596_7 | |
| 328333 | EOS28264 | c_7_hs gi\|5868375\|ref\|gn 6 + 282506 282664 ex 4 5 CDSi 7.71 159 517 | | | 1.5 |
| | | | | CH.07_hs gi\|5868375 | |
| 304143 | EOS04074 | R88737 | | EST singleton (not in UniGene) with exon hit | 1.5 |
| 329625 | EOS29556 | c11_p2 gi\|4567169\|gb\|A gn 2 − 85893 85984 ex 3 5 CDSi 2.24 92 29 | | | 1.5 |
| | | | | CH.11_p2 gi\|4567169 | |
| 329960 | EOS29891 | c16_p2 gi\|5091594\|gb\|A gn 1 − 1031 1162 ex 1 3 CDSi 10.75 132 415 | | | 1.5 |
| | | | | CH.16_p2 gi\|5091594 | |
| 318975 | EOS18906 | Z44110 | | EST cluster (not in UniGene) | 1.5 |
| 321875 | EOS21806 | N49122 | | EST cluster (not in UniGene) | 1.5 |
| 320451 | EOS20382 | R26944 | Hs.180777 | Homo sapiens mRNA; cDNA DKFZp564M0264 (from clone DKFZp564M0264) | 1.5 |
| 336020 | EOS35951 | CH22_3403FG_669_9_LINK_DJ32I10.GENSCAN.9-14 | | | 1.5 |
| | | | | CH22_FGENES.669_9 | |
| 332581 | EOS32512 | T28799 | Hs.2913 | EphB3 | 1.5 |
| 338622 | EOS38553 | CH22_7384FG_LINK_EM:AC005500.GENSCAN.451-1 | | | 1.5 |
| | | | | CH22_EM:AC005500.GENSCAN.451-1 | |
| 330397 | EOS30328 | D14659 | Hs.154387 | KIAA0103 gene product | 1.5 |
| 314359 | EOS14290 | AA205569 | Hs.194193 | ESTs | 1.5 |
| 313456 | EOS13387 | AW380579 | Hs.209657 | ESTs | 1.5 |
| 318486 | EOS18417 | H09123 | Hs.139258 | ESTs | 1.5 |
| 318175 | EOS18106 | AA644624 | | EST cluster (not in UniGene) | 1.5 |
| 335684 | EOS35615 | CH22_3045FG_595_4_LINK_EM:AC005500.GENSCAN.487-13 | | | 1.5 |
| | | | | CH22_FGENES.595_4 | |
| 327814 | EOS27745 | c_5_hs gi\|5867968\|ref\|gn 6 + 69377 70566 ex 1 2 CDSf 86.15 1190 999 | | | 1.5 |
| | | | | CH.05_hs gi\|5867968 | |
| 322120 | EOS22051 | W84351 | Hs.213846 | ESTs | 1.5 |
| 311749 | EOS11680 | R06249 | Hs.13911 | ESTs | 1.5 |
| 329797 | EOS29728 | c14_p2 gi\|6523160\|emb\|gn 1 − 10616 10894 ex 3 6 CDSi 5.86 279 1549 | | | 1.5 |
| | | | | CH.14_p2 gi\|6523160 | |
| 330630 | EOS30561 | X78669 | Hs.79088 | reticulocalbin 2; EF-hand calcium binding domain | 1.5 |
| 303777 | EOS03708 | AA348491 | | EST cluster (not in UniGene) with exon hit | 1.5 |
| 309656 | EOS09587 | AW197060 | Hs.195188 | glyceraldehyde-3-phosphate dehydrogenase | 1.5 |
| 326165 | EOS26096 | c17_hs gi\|5867208\|ref\|gn 2 − 62787 62929 ex 1 10 CDSl 0.87 143 2037 | | | 1.5 |
| | | | | CH.17_hs gi\|5867208 | |
| 308328 | EOS08259 | AI590571 | Hs.186412 | EST | 1.5 |
| 300601 | EOS00532 | AI762130 | Hs.165619 | ESTs | 1.5 |
| 303610 | EOS03541 | AA323288 | | EST cluster (not in UniGene) with exon hit | 1.5 |
| 307856 | EOS07787 | AI366158 | | EST singleton (not in UniGene) with exon hit | 1.5 |
| 319920 | EOS19851 | R54575 | Hs.13337 | ESTs; Weakly similar to similar to Phosphoglucomutase and phosphomannomutase | 1.5 |
| | | | | phosphoserine [C.elegans] | |
| 332167 | EOS32098 | D57389 | Hs.75447 | ralA binding protein 1 | 1.5 |
| 316427 | EOS16358 | AI241019 | Hs.145644 | ESTs | 1.5 |
| 303886 | EOS03817 | AW365963 | | EST cluster (not in UniGene) with exon hit | 1.5 |
| 314292 | EOS14223 | AA732590 | Hs.134740 | ESTs | 1.5 |
| 315408 | EOS15339 | AW273261 | Hs.216292 | ESTs | 1.5 |
| 335698 | EOS35629 | CH22_3059FG_597_1_LINK_EM:AC005500.GENSCAN.489-1 | | | 1.5 |
| | | | | CH22_FGENES.597_1 | |
| 315084 | EOS15015 | AI821085 | Hs.187796 | ESTs | 1.5 |
| 302299 | EOS02230 | R64632 | Hs.182167 | hemoglobin; gamma A | 1.5 |
| 306803 | EOS06734 | AI055860 | Hs.193717 | interleukin 10 | 1.5 |
| 315802 | EOS15733 | AA677540 | Hs.117064 | ESTs | 1.5 |
| 326257 | EOS26188 | c17_hs gi\|5867264\|ref\|gn 6 + 222712 222819 ex 2 2 CDSl 4.46 108 3597 | | | 1.5 |
| | | | | CH.17_hs gi\|5867264 | |
| 319599 | EOS19530 | H56112 | | EST cluster (not in UniGene) | 1.5 |
| 321891 | EOS21822 | AW157424 | Hs.165954 | ESTs | 1.5 |
| 335164 | EOS35095 | CH22_2500FG_502_8_LINK_EM:AC005500.GENSCAN.396-23 | | | 1.5 |
| | | | | CH22_FGENES.502_8 | |
| 327133 | EOS27064 | c21_hs gi\|6682522\|ref\|gn 1 + 38069 38938 ex 2 2 CDSl 63.42 870 1583 | | | 1.5 |
| | | | | CH.21_hs gi\|6682522 | |
| 317460 | EOS17391 | AA926980 | Hs.131347 | ESTs | 1.5 |
| 332344 | EOS32275 | W45574 | Hs.252497 | ESTs | 1.5 |
| 328801 | EOS28732 | c_7_hs gi\|5868321\|ref\|gn 1 − 44492 44609 ex 2 3 CDSi 1.71 118 5525 | | | 1.5 |
| | | | | CH.07_hs gi\|5868321 | |
| 321677 | EOS21608 | N44545 | Hs.251865 | ESTs | 1.5 |
| 331858 | EOS31789 | AA421163 | Hs.163848 | ESTs | 1.5 |
| 309243 | EOS09174 | AI972052 | | EST singleton (not in UniGene) with exon hit | 1.5 |
| 326213 | EOS26144 | c17_hs gi\|5867224\|ref\|gn 3 − 60751 60927 ex 1 4 CDSl 2.06 177 2687 | | | 1.5 |
| | | | | CH.17_hs gi\|5867224 | |
| 321632 | EOS21563 | AA419617 | | EST cluster (not in UniGene) | 1.5 |
| 321424 | EOS21355 | AA057301 | | EST cluster (not in UniGene) | 1.5 |
| 322465 | EOS22396 | AA137152 | Hs.3784 | ESTs; Highly similar to phosphoserine aminotransferase [H. sapiens] | 1.5 |
| 333391 | EOS33322 | CH22_637FG_144_6_LINK_EM:AC005500.GENSCAN.25-6 | | | 1.5 |
| | | | | CH22_FGENES.144_6 | |
| 333384 | EOS33315 | CH22_630FG_143_23_LINK_EM:AC005500.GENSCAN.24-17 | | | 1.5 |
| | | | | CH22_FGENES.143_23 | |
| 334784 | EOS34715 | CH22_2096FG_432_9_LINK_EM:AC005500.GENSCAN.293-12 | | | 1.5 |
| | | | | CH22_FGENES.432_9 | |
| 334078 | EOS34009 | CH22_1356FG_327_33_LINK_EM:AC005500.GENSCAN.181-35 | | | 1.5 |
| | | | | CH22_FGENES.327_33 | |
| 335158 | EOS35089 | CH22_2494FG_502_2_LINK_EM:AC005500.GENSCAN.396-17 | | | 1.5 |
| | | | | CH22_FGENES.502_2 | |
| 335062 | EOS34993 | CH22_2388FG_482_17_LINK_EM:AC005500.GENSCAN.376-16 | | | 1.5 |
| | | | | CH22_FGENES.482_17 | |
| 333243 | EOS33174 | CN22_482FG_111_7_LINK_EM.AC000097.GENSCAN.120-6 | | | 1.5 |
| | | | | CH22_FGENES.111_7 | |
| 306380 | EOS06311 | AA968861 | | EST singleton (not in UniGene) with exon hit | 1.5 |
| 320809 | EOS20740 | AI540299 | | EST cluster (not in UniGene) | 1.5 |
| 332813 | EOS32744 | CH22_29FG_8_1_LINK_C65E1.GENSCAN.2-2 | | | 1.5 |
| | | | | CH22_FGENES.8_1 | |
| 335817 | EOS35748 | CH22_3189FG_618_5_LINK_EM:AC005500.GENSCAN.510-5 | | | 1.5 |
| | | | | CH22_FGENES.618_5 | |
| 319551 | EOS19482 | AA761668 | | EST cluster (not in UniGene) | 1.5 |
| 334472 | EOS34403 | CH22_1771FG_394_3_LINK_EM:AC005500.GENSCAN.257-3 | | | 1.5 |
| | | | | CH22_FGENES.394.3 | |
| 333029 | EOS32960 | CH22_255FG_68_3_LINK_EM:AC000097.GENSCAN.40-3 | | | 1.5 |
| | | | | CH22_FGENES.68_3 | |
| 308055 | EOS07986 | AI468091 | Hs.119252 | tumor protein; translationally-controlled 1 | 1.5 |
| 302882 | EOS02813 | AW403330 | | EST cluster (not in UniGene) with exon hit | 1.5 |
| 314033 | EOS13964 | AA167125 | | EST cluster (not in UniGene) | 1.5 |
| 324928 | EOS24859 | AI932285 | Hs.160569 | ESTs | 1.5 |
| 329524 | EOS29455 | c10_p2 gi\|3983507\|gb\|A gn − 38025 38143 ex 3 3 CDSi 2.40 119 170 | | | 1.5 |
| | | | | CH.10_p2 gi\|3983507 | |
| 333131 | EOS33062 | CH22_360FG_83_6_LINK_EM:AC000097.GENSCAN.67-10 | | | 1.5 |
| | | | | CH22_FGENES.83_6 | |
| 332085 | EOS32016 | AA600353 | Hs.173933 | ESTs; Weakly similar to NUCLEAR FACTOR 1/X [H. sapiens] | 1.5 |
| 305369 | EOS05300 | AA714040 | | EST singleton (not in UniGene) with exon hit | 1.5 |
| 300344 | EOS00275 | AW291487 | Hs.213659 | ESTs | 1.5 |
| 325071 | EOS25002 | H09693 | | EST cluster (not in UniGene) | 1.5 |
| 323693 | EOS23624 | AW297758 | Hs.249721 | ESTs | 1.5 |
| 321899 | EOS21830 | N55158 | Hs.135252 | ESTs | 1.5 |
| 331857 | EOS31788 | AA421160 | Hs.9456 | SWI/SNF related; matrix associated; actin dependent regulator of | 1.5 |
| | | | | chromatin; subfamily a; member 5 | |
| 334850 | EOS34781 | CH22_2164FG_439_36_LINK_EM:AC005500.GENSCAN.311-13 | | | 1.5 |
| | | | | CH22_FGENES.439_36 | |
| 322610 | EOS22541 | AF180919 | | EST cluster (not in UniGene) | 1.5 |
| 335332 | EOS35263 | CH22_2677FG_535_6_LINK_EM:AC005500.GENSCAN.426-6 | | | 1.5 |
| | | | | CH22_FGENES.535_6 | |
| 307565 | EOS07496 | AI282468 | | EST singleton (not in UniGene) with exon hit | 1.5 |
| 314140 | EOS14071 | AI216473 | Hs.154297 | ESTs | 1.5 |
| 323011 | EOS22942 | AA580288 | | EST cluster (not in UniGene) | 1.5 |
| 325366 | EOS25297 | c12_hs gi\|5866920\|ref\|gn 9 -920962 921713 ex 1 8 CDSl 15.95 752 167 | | | 1.5 |
| | | | | CH.12_hs gi\|5866920 | |
| 322306 | EOS22237 | W75935 | Hs.146083 | ESTs | 1.5 |
| 311034 | EOS10965 | AI564023 | Hs.171467 | ESTs; Highly similar to NKG2-D TYPE II INTEGRAL | 1.5 |
| | | | | MEMBRANE PROTEIN [H. sapiens] | |
| 305081 | EOS05012 | AA641638 | | EST singleton (not in UniGene) with exon hit | 1.5 |
| 322933 | EOS22864 | AA099759 | | EST cluster (not in UniGene) | 1.5 |
| 335221 | EOS35152 | CH22_2560FG_513_4_LINK_EM:AC005500.GENSCAN.406-4 | | | 1.5 |
| | | | | CH22_FGENES.513_4 | |
| 304948 | EOS04879 | AA613107 | | EST singleton (not in UniGene) with exon hit | 1.5 |
| 334900 | EOS34831 | CH22_2217FG_452_14_LINK_EM:AC005500.GENSCAN.341-17 | | | 1.5 |
| | | | | CH22_FGENES.452_14 | |
| 318404 | EOS18335 | AI654108 | Hs.135125 | ESTs | 1.5 |
| 339358 | EOS39289 | CH22_8328FG_LINK_BA354I12.GENSCAN.31-3 | | | 1.5 |
| | | | | CH22_BA354I12.GENSCAN.31-3 | |
| 327074 | EOS27005 | c21_hs gi\|6531965\|ref\|gn 58 + 4039993 4040096 ex 3 4 CDSi 0.68 104 1284 | | | 1.5 |
| | | | | CH.21_hs gi\|6531965 | |
| 326054 | EOS25985 | c17_hs gi\|5867184\|ref\|gn 2 − 146342 146469 x 3 4 CDSi 10.00 128 426 | | | 1.5 |
| | | | | CH.17_hs gi\|5867184 | |
| 326892 | EOS26823 | c20_hs gi\|6682511\|ref\|gn 5 + 119424 119500 ex 29 30 CDSi 18.89 77 2313 | | | 1.5 |
| | | | | CH.20_hs gi\|6682511 | |
| 328767 | EOS28698 | c_7_hs gi\|6017031\|ref\|gn 1 − 35625 35723 ex 4 4 CDSf 5.63 99 5262 | | | 1.5 |
| | | | | CH.07_hs gi\|6017031 | |
| 337772 | EOS37703 | CH22_6125FG_LINK_EM:AC000097.GENSCAN.119-11 | | | 1.5 |
| | | | | CH22_EM:AC000097.GENSCAN.119-11 | |
| 312199 | EOS12130 | AW438602 | Hs.191179 | ESTs | 1.5 |
| 303506 | EOS03437 | AA340605 | Hs.105887 | ESTs | 1.5 |
| 325176 | EOS25107 | T52843 | | EST cluster (not in UniGene) | 1.5 |
| 302023 | EOS01984 | AF060567 | Hs.126782 | sushi-repeat protein | 1.5 |
| 305833 | EOS05764 | AA857836 | Hs.181165 | eukaryotic translation elongation factor 1 alpha 1 | 1.5 |
| 309131 | EOS09062 | AI929175 | Hs.119122 | ribosomal protein L13a | 1.5 |
| 334184 | EOS34115 | CH22_1465FG_350_15_LINK_EM:AC005500.GENSCAN.209-17 | | | 1.5 |
| | | | | CH22_FGENES.350_15 | |
| 335188 | EOS35119 | CH22_2524FG_507_3_LINK_EM:AC005500.GENSCAN.400-3 | | | 1.5 |
| | | | | CH22_FGENES.507_3 | |
| 304813 | EOS04744 | AA584540 | | EST singleton (not in UniGene) with exon hit | 1.5 |
| 315359 | EOS15290 | AA608808 | Hs.225118 | ESTs | 1.5 |
| 324434 | EOS24365 | AA707249 | Hs.98789 | ESTs | 1.5 |
| 327910 | EOS27841 | c_6_hs gi\|5868162\|ref\|gn 1 + 21622 21748 ex 6 7 CDSi 3.69 127 449 | | | 1.4 |
| | | | | CH.06_hs gi\|5868162 | |
| 335671 | EOS35602 | CH22_3031FG_592_3_LINK_EM:AC005500.GENSCAN.485-4 | | | 1.4 |
| | | | | CH22_FGENES.592_3 | |
| 334943 | EOS34874 | CH22_2264FG_465_8_LINK_EM:AC005500.GENSCAN.359-8 | | | 1.4 |
| | | | | CH22_FGENES.465_8 | |
| 326393 | EOS26324 | c19_hs gi\|5867341\|ref\|gn 2 + 41702 41841 ex 5 5 CDSi 20.15 140 504 | | | 1.4 |
| | | | | CH.19_hs gi\|5867341 | |
| 305296 | EOS05227 | AA687181 | | EST singleton (not in UniGene) with exon hit | 1.4 |
| 307243 | EOS07174 | AI199957 | | EST singleton (not in UniGene) with exon hit | 1.4 |
| 320066 | EOS19997 | AW364885 | Hs.112442 | ESTs | 1.4 |
| 311465 | EOS11396 | AI758660 | Hs.206132 | ESTs | 1.4 |
| 302822 | EOS02753 | AW404176 | Hs.111611 | ribosomal protein L27 | 1.4 |
| 304987 | EOS04918 | AA618044 | | EST singleton (not in UniGene) with exon hit | 1.4 |
| 330892 | EOS30823 | AA149579 | Hs.118258 | ESTs | 1.4 |
| 333385 | EOS33316 | CH22_631FG_143_24_LINK_EM:AC005500.GENSCAN.24-18 | | | 1.4 |
| | | | | CH22_FGENES.143_24 | |
| 302626 | EOS02557 | AB021870 | | EST cluster (not in UniGene) with exon hit | 1.4 |
| 318042 | EOS17973 | AW294522 | Hs.149991 | ESTs | 1.4 |
| 339361 | EOS39292 | CH22_8331FG_LINK_BA354I12.GENSCAN.32-3 | | | 1.4 |
| | | | | CH22_BA354112.GENSCAN.32-3 | |
| 309000 | EOS08931 | AI880489 | | EST singleton (not in UniGene) with exon hit | 1.4 |
| 306004 | EOS05935 | AA889992 | | EST singleton (not in UniGene) with exon hit | 1.4 |
| 329539 | EOS29470 | c10_p2 gi\|3983503\|gb\|U gn 1 − 1 326 ex 1 3 CDSl 41.66 326 212 | | | 1.4 |
| | | | | CH.10_p2 gi\|3983503 | |
| 313663 | EOS13594 | AI953261 | Hs.169813 | ESTs | 1.4 |
| 323538 | EOS23469 | AW247696 | | EST cluster (not in UniGene) | 1.4 |
| 337595 | EOS37526 | CH22_5884FG_LINK_C20H12.GENSCAN.8-1 | | | 1.4 |
| | | | | CH22_C20H12.GENSCAN.8-1 | |
| 303149 | EOS03080 | AA312995 | | EST cluster (not in UniGene) with exon hit | 1.4 |
| 308484 | EOS08415 | AI679292 | | EST singleton (not in UniGene) with exon hit | 1.4 |
| 300912 | EOS00843 | AW138724 | Hs.168974 | ESTs | 1.4 |
| 315158 | EOS15089 | AA744438 | Hs.142476 | ESTs; Weakly similar to !!!! ALU CLASS D WARNING ENTRY !!!! [H. sapiens] | 1.4 |
| 300462 | EOS00393 | AA746501 | Hs.14217 | ESTs | 1.4 |
| 312730 | EOS12661 | AI804372 | Hs.208661 | ESTs | 1.4 |
| 316868 | EOS16799 | AI660898 | Hs.195602 | ESTs | 1.4 |
| 337629 | EOS37560 | CH22_5933FG_LINK_C20H12.GENSCAN.28-35 | | | 1.4 |
| | | | | CH22_C20H12.GENSCAN.28-35 | |
| 332518 | EOS32449 | D16562 | Hs.155433 | ATP synthase; H + transporting; mitochondrial F1 complex; gamma polypeptide 1 | 1.4 |
| 337422 | EOS37353 | CH22_{—} | | CH22_FGENES.760-2 | 1.4 |
| | | 5624FG_760_2_{—} | | | |
| 328835 | EOS28766 | c_7_hs gi\|5868339\|ref\|gn 5 + 88053 88461 ex 3 3 CDSl 13.78 409 5775 | | | 1.4 |
| | | | | CH.07_hs gi\|5868339 | |
| 338282 | EOS38213 | CH22_6897FG_LINK_EM:AC005500.GENSCAN.291-4 | | | 1.4 |
| | | | | CH22_EM:AC005500.GENSCAN.291-4 | |
| 337895 | EOS37826 | CH22_6303FG_LINK_EM:AC005500.GENSCAN.56-2 | | | 1.4 |
| | | | | CH22_EM:AC005500.GENSCAN.56-2 | |
| 320330 | EOS20261 | AF026004 | Hs.141660 | chloride channel 2 | 1.4 |
| 314302 | EOS14233 | AA813118 | Hs.163230 | ESTs | 1.4 |
| 313280 | EOS13211 | AI285537 | Hs.222830 | ESTs | 1.4 |
| 333222 | EOS33153 | CH22_459FG_105_2_LINK_EM:AC000097.GENSCAN.109-6 | | | 1.4 |
| | | | | CH22_FGENES.105_2 | |
| 305726 | EOS05657 | AA828156 | | EST singleton (not in UniGene) with exon hit | 1.4 |
| 312674 | EOS12605 | AI762475 | Hs.151327 | ESTs; Moderately similar to !!!! ALU SUBFAMILY J WARNING | 1.4 |
| | | | | ENTRY !!!! [H. sapiens] | |
| 315869 | EOS15800 | AI033547 | Hs.132826 | ESTs | 1.4 |
| 327010 | EOS26941 | c21_hs gi\|5867664\|ref\|gn 12 + 941057 941139 ex 9 9 CDSl 7.44 83 790 | | | 1.4 |
| | | | | CH.21_hs gi\|5867664 | |
| 325892 | EOS25823 | c16_hs gi\|5867088\|ref\|gn 1 − 10498 10652 ex 2 3 CDSi 3.94 155 870 | | | 1.4 |
| | | | | CH.16_hs gi\|5867088 | |
| 302575 | EOS02506 | AF071164 | Hs.249171 | homeo box A11 | 1.4 |
| 301970 | EOS01901 | AB028962 | Hs.120245 | KIAA1039 protein | 1.4 |
| 332207 | EOS32138 | H61475 | Hs.237353 | EST | 1.4 |
| 316024 | EOS15955 | AA707141 | Hs.193388 | ESTs | 1.4 |
| 314599 | EOS14530 | AW206512 | Hs.186996 | ESTs | 1.4 |
| 333585 | EOS33516 | CH22_846FG_203_4_LINK_EM:AC005500.GENSCAN.74-6 | | | 1.4 |
| | | | | CH22_FGENES.203_4 | |
| 324670 | EOS24601 | AI525557 | | EST cluster (not in UniGene) | 1.4 |
| 321307 | EOS21238 | R85409 | | EST cluster (not in UniGene) | 1.4 |
| 335170 | EOS35101 | CH22_2506FG_503_1_LINK_EM:AC005500.GENSCAN.397-1 | | | 1.4 |
| | | | | CH22_FGENES.503_1 | |
| 328274 | EOS28205 | c_7_hs gi\|5868219\|ref\|gn 2 − 31244 31439 ex 111 CDSl 13.06 196 9 | | | 1.4 |
| | | | | CH.07_hs gi\|5868219 | |
| 336880 | EOS36811 | CH22_4619FG_{—} | | CH22_FGENES.318-8 | 1.4 |
| | | 318_8_{—} | | | |
| 313825 | EOS13756 | AA215470 | | EST cluster (not in UniGene) | 1.4 |
| 318410 | EOS18341 | AI138418 | Hs.144935 | ESTs | 1.4 |
| 335361 | EOS35292 | CH22_2710FG_541_11_LINK_EM:AC005500.GENSCAN.431-16 | | | 1.4 |
| | | | | CH22_FGENES.541_11 | 1.4 |
| 319802 | EOS19733 | AI701489 | Hs.202501 | ESTs | 1.4 |
| 334769 | EOS34700 | CH22_2081FG_429_4_LINK_EM:AC005500.GENSCAN.290-9 | | | 1.4 |
| | | | | CH22_FGENES.429_4 | |
| 312709 | EOS12640 | AW069181 | Hs.141146 | ESTs; Weakly similar to transformation-related protein [H. sapiens] | 1.4 |
| 330004 | EOS29935 | c16_p2 gi\|6623963\|gb\|A gn 5 − 78872 78999 ex 2 6 CDSi 19.93 128 728 | | | 1.4 |
| | | | | CH.16_p2 gi\|6623963 | |
| 313103 | EOS13034 | AI184303 | Hs.143806 | ESTs | 1.4 |
| 326359 | EOS26290 | c18_hs gi\|5867293\|ref\|gn 1 + 9436 9494 ex 2 3 CDSi 2.16 59 88 | | | 1.4 |
| | | | | CH.18_hs gi\|5867293 | |
| 305211 | EOS05142 | AA668563 | | EST singleton (not in UniGene) with exon hit | 1.4 |
| 334628 | EOS34559 | CH22_1936FG_416_4_LINK_EM:AC005500.GENSCAN.277-4 | | | 1.4 |
| | | | | CH22_FGENES.416_4 | |
| 326919 | EOS26850 | c21_hs gi\|6456782\|ref\|gn 2 − 40486 41046 ex 1 5 CDSl 17.70 561 157 | | | 1.4 |
| | | | | CH.21_hs gi\|6456782 | |
| 315527 | EOS15458 | AI791138 | Hs.116768 | ESTs | 1.4 |
| 306090 | EOS06021 | AA908609 | | EST singleton (not in UniGene) with exon hit | 1.4 |
| 303316 | EOS03247 | AF033122 | Hs.14125 | p53 regulated PA26 nuclear protein | 1.4 |
| 303642 | EOS03573 | AW299459 | | EST cluster (not in UniGene) with exon hit | 1.4 |
| 314357 | EOS14288 | AA781795 | Hs.122587 | ESTs | 1.4 |
| 337102 | EOS37033 | CH22_5033FG_{—} | | CH22_FGENES.472-7 | 1.4 |
| | | 472_7_{—} | | | |
| 304384 | EOS04315 | AA235482 | Hs.62954 | ferritin; heavy polypeptide 1 | 1.4 |
| 315117 | EOS15048 | AA828609 | Hs.192044 | ESTs | 1.4 |
| 305750 | EOS05681 | AA835250 | | EST singleton (not in UniGene) with exon hit | 1.4 |
| 311726 | EOS11657 | AW081766 | Hs.253920 | ESTs | 1.4 |
| 326996 | EOS26927 | c21_hs gi\|5867660\|ref\|gn 4 − 63212 63404 ex 2 6 CDSi 15.70 193 622 | | | 1.4 |
| | | | | CH.21_hs gi\|5867660 | |
| 330257 | EOS30188 | c_5_p2 gi\|6671881\|gb\|A gn 2 − 143228 143393 ex 1 9 CDSl 11.31 166 586 | | | 1.4 |
| | | | | CH.05_p2 gi\|6671881 | 1.4 |
| 323864 | EOS23795 | AA340724 | Hs.214028 | ESTs | 1.4 |
| 338204 | EOS38135 | CH22_6773FG_LINK_EM:AC005500.GENSCAN.241-3 | | | 1.4 |
| | | | | CH22_EM:AC005500.GENSCAN.241-3 | |
| 314025 | EOS13956 | AI983981 | Hs.189114 | ESTs | 1.4 |
| 315974 | EOS15905 | AW029203 | Hs.191952 | ESTs | 1.4 |
| 335599 | EOS35530 | CH22_2957FG_581_39_LINK_EM:AC005500.GENSCAN.476-37 | | | 1.4 |
| | | | | CH22_FGENES.581_39 | |
| 335364 | EOS35295 | CH22_2713FG_543_2_LINK_EM:AC005500.GENSCAN.432-4 | | | 1.4 |
| | | | | CH22_FGENES.543_2 | |
| 303634 | EOS03565 | AI953377 | Hs.169425 | ESTs; Weakly similar to predicted using Genefinder [C.elegans] | 1.4 |
| 315626 | EOS15557 | AA808598 | Hs.35353 | ESTs; Weakly similar to H21P03.2 [C.elegans] | 1.4 |
| 329936 | EOS29867 | c16_p2 gi\|6165200\|gb\|A gn 4 − 82761 82920 ex 3 4 CDSi 1.15 160 199 | | | 1.4 |
| | | | | CH.16_p2 gi\|6165200 | |
| 328632 | EOS28563 | c_7_hs gi\|5868247\|ref\|gn 1 + 76734 76853 ex 1 4 CDSf 13.95 120 3764 | | | 1.4 |
| | | | | CH.07_hs gi\|5868247 | |
| 330207 | EOS30138 | c_5_p2 gi\|6013606\|gb\|A gn 3 − 109912 110004 ex 2 4 CDSi 6.54 93 174 | | | 1.4 |
| | | | | CH.05_p2 gi\|6013606 | |
| 329919 | EOS29850 | c16_p2 gi\|6223624\|gb\|A gn 6 − 103492 103681 ex 1 8 CDSl 6.18 190 93 | | | 1.4 |
| | | | | CH.16_p2 gi\|6223624 | |
| 331916 | EOS31847 | AA446131 | Hs.124918 | ESTs | 1.4 |
| 317617 | EOS17548 | T58194 | | EST cluster (not in UniGene) | 1.4 |
| 331943 | EOS31874 | AA453418 | Hs.178272 | ESTs | 1.4 |
| 306413 | EOS06344 | AA973288 | | EST singleton (not in UniGene) with exon hit | 1.4 |
| 313607 | EOS13538 | N94169 | Hs.194258 | ESTs; Moderately similar to !!!! ALU SUBFAMILY SC WARNING | 1.4 |
| | | | | ENTRY !!!! [H. sapiens] | |
| 336292 | EOS36223 | CH22_3691FG_783_3_LINK_BA354I12.GENSCAN.4-7 | | | 1.4 |
| | | | | CH22_FGENES.783_3 | |
| 330453 | EOS30384 | HG3976-HT4246 | | Pou-Domain Dna Binding Factor Pit1, Pituitary-Specific | 1.4 |
| 324602 | EOS24533 | AA503620 | Hs.213239 | ESTs | 1.4 |
| 332183 | EOS32114 | H08225 | Hs.177181 | ESTs | 1.4 |
| 320032 | EOS19963 | AI699772 | Hs.202361 | ESTs; Weakly similar to X-linked retinopathy protein [H. sapiens] | 1.4 |
| 333156 | EOS33087 | CH22_387FG_89_6_LINK_EM:AC000097.GENSCAN.84-8 | | | 1.4 |
| | | | | CH22_FGENES.89_6 | |
| 334156 | EOS34087 | CH22_1435FG_340_6_LINK_EM:AC005500.GENSCAN.190-7 | | | 1.4 |
| | | | | CH22_FGENES.340_6 | |
| 334303 | EOS34234 | CH22_1594FG_373_6_LINK_EM:AC005500.GENSCAN.233-5 | | | 1.4 |
| | | | | CH22_FGENES.373_6 | |
| 325513 | EOS25444 | c12_hs gi\|6017035\|ref\|gn 1 − 34295 34490 ex 2 7 CDSi 6.49 196 2471 | | | 1.4 |
| | | | | CH.12_hs gi\|6017035 | |
| 302758 | EOS02689 | AA984563 | | EST cluster (not in UniGene) with exon hit | 1.4 |
| 329557 | EOS29488 | c10_p2 gi\|3962492\|gb\|A gn 6 − 53197 53647 ex 2 2 CDSf 37.68 451 247 | | | 1.4 |
| | | | | CH.10_p2 gi\|3962492 | |
| 331717 | EOS31648 | AA190888 | Hs.153881 | ESTs; Highly similar to NY-REN-62 antigen [H. sapiens] | 1.4 |
| 325885 | EOS25816 | c16_hs gi\|5867087\|ref\|gn 11 + 193212 193377 ex 1 3 CDSf 43.19 166 792 | | | 1.4 |
| | | | | CH.16_hs gi\|5867087 | |
| 312160 | EOS12091 | AA805903 | Hs.184371 | ESTs | 1.4 |
| 328882 | EOS28813 | c_7_hs gi\|6552423\|ref\|gn 2 − 157669 157826 ex 4 6 CDSi 4.91 158 6200 | | | 1.4 |
| | | | | CH.07_hs gi\|6552423 | |
| 339028 | EOS38959 | CH22_7925FG_LINK_DA59H18.GENSCAN.22-8 | | | 1.4 |
| | | | | CH22_DA59H18.GENSCAN.22-8 | |
| 323497 | EOS23428 | AI523613 | Hs.221544 | ESTs | 1.4 |
| 316897 | EOS16828 | AA838114 | | EST cluster (not in UniGene) | 1.4 |
| 312479 | EOS12410 | AI950844 | Hs.128738 | ESTs; Weakly similar to non-lens beta gamma-crystallin like protein [H. sapiens] | 1.4 |
| 338535 | EOS38466 | CH22_7251FG_LINK_EM:AC005500.GENSCAN.404-3 | | | 1.4 |
| | | | | CH22_EM:AC005500.GENSCAN.404-3 | |
| 312754 | EOS12685 | R99834 | Hs.250383 | ESTs | 1.4 |
| 327527 | EOS27458 | c_2_hs gi\|6381882\|ref\|gn 2 − 98950 99040 ex 4 8 CDSi 5.78 91 1768 | | | 1.4 |
| | | | | CH.02_hs gi\|6381882 | |
| 324714 | EOS24645 | AA574312 | Hs.245737 | ESTs | 1.4 |
| 302347 | EOS02278 | AF039400 | Hs.194659 | chloride channel; calcium activated; family member 1 | 1.4 |
| 338008 | EOS37939 | CH22_6490FG_LINK_EM:AC005500.GENSCAN.127-9 | | | 1.4 |
| | | | | CH22_EM:AC005500.GENSCAN.127-9 | |
| 315590 | EOS15521 | AA640637 | Hs.225817 | ESTs | 1.4 |
| 320825 | EOS20756 | NM_004751 | | EST cluster (not in UniGene) | 1.4 |
| 300930 | EOS00861 | AI289481 | Hs.136371 | ESTs | 1.4 |
| 335225 | EOS35156 | CH22_2564FG_513_10_LINK_EM:AC005500.GENSCAN.406-9 | | | 1.4 |
| | | | | CH22_FGENES.513_10 | |
| 337303 | EOS37234 | CH22_5442FG_{—} | | CH22_FGENES.681-5 | 1.4 |
| | | 681_5_{—} | | | |
| 317198 | EOS17129 | AI810384 | Hs.128025 | ESTs | 1.4 |
| 308991 | EOS08922 | AI879831 | | EST singleton (not in UniGene) with exon hit | 1.4 |
| 325472 | EOS25403 | c12_hs gi\|6017034\|ref\|gn 7 − 289581 289657 ex 2 6 CDSi 4.74 77 1786 | | | 1.4 |
| | | | | CH.12_hs gi\|6017034 | |
| 301266 | EOS01197 | AA829774 | | EST cluster (not in UniGene) with exon hit | 1.4 |
| 330901 | EOS30832 | AA157818 | Hs.238380 | Human endogenous retroviral protease mRNA; complete cds | 1.4 |
| 313406 | EOS13337 | AI248314 | Hs.132932 | ESTs | 1.4 |
| 301454 | EOS01385 | AI751738 | | EST cluster (not in UniGene) with exon hit | 1.4 |
| 317269 | EOS17200 | AA906411 | Hs.127378 | ESTs | 1.4 |
| 338876 | EOS38807 | CH22_7733FG_LINK_DJ32I10.GENSCAN.4-2 | | | 1.4 |
| | | | | CH22_DJ32I10.GENSCAN.4-2 | |
| 328481 | EOS28412 | c_7_hs gi\|5868449\|ref\|gn 1 − 8987 9180 ex 4 31 CDSi 10.00 194 2103 | | | 1.4 |
| | | | | CH.07_hs gi\|5868449 | |
| 314022 | EOS13953 | AW452420 | Hs.248678 | ESTs | 1.4 |
| 307640 | EOS07571 | AI301992 | | EST singleton (not in UniGene) with exon hit | 1.4 |
| 315541 | EOS15472 | AI168233 | Hs.123159 | ESTs; Weakly similar to KIAA0668 protein [H. sapiens] | 1.4 |
| 315489 | EOS15420 | AA628245 | Hs.191847 | ESTs | 1.4 |
| 327815 | EOS27746 | c_5_hs gi\|5867968\|ref\|gn 6 + 70804 71401 ex 2 2 CDSl 27.99 598 1000 | | | 1.4 |
| | | | | CH.05_hs gi\|5867968 | |
| 339319 | EOS39250 | CH22_8280FG_LINK_BA354I12.GENSCAN.22-19 | | | 1.4 |
| | | | | CH22_BA354I12.GENSCAN.22-19 | |
| 322564 | EOS22495 | W86440 | Hs.118344 | ESTs | 1.4 |
| 323812 | EOS23743 | AW081373 | Hs.199199 | ESTs | 1.4 |
| 303540 | EOS03471 | AA355607 | Hs.173590 | ESTs; Weakly similar to MMSET type I [H. sapiens] | 1.4 |
| 337902 | EOS37833 | CH22_6314FG_LINK_EM:AC005500.GENSCAN.56-13 | | | 1.4 |
| | | | | CH22_EM:AC005500.GENSCAN.56-13 | |
| 335289 | EOS35220 | CH22_2631FG_527_2_LINK_EM:AC005500.GENSCAN.421-2 | | | 1.4 |
| | | | | CH22_FGENES.527_2 | |
| 327919 | EOS27850 | c_6_hs gi\|5868165\|ref\|gn 6 + 547701 547800 ex 14 14 CDSl − 0.20 100 505 | | | 1.4 |
| | | | | CH.06_hs gi\|5868165 | |
| 337674 | EOS37605 | CH22_6005FG_LINK_EM:AC000097.GENSCAN.67-4 | | | 1.4 |
| | | | | CH22_EM:AC000097.GENSCAN.67-4 | |
| 320087 | EOS20018 | AF032387 | Hs.113265 | small nuclear RNA activating complex; polypeptide 4; 190 kD | 1.4 |
| 334939 | EOS34870 | CH22_2259FG_465_3_LINK_EM:AC005500.GENSCAN.359-3 | | | 1.3 |
| | | | | CH22_FGENES.465_3 | |
| 303443 | EOS03374 | AA320525 | | EST cluster (not in UniGene) with exon hit | 1.3 |
| 325929 | EOS25860 | c16_hs gi\|5867125\|ref\|gn 2 − 51715 51996 ex 1 1 CDSo 29.05 282 1594 | | | 1.3 |
| | | | | CH.16_hs gi\|5867125 | |
| 327745 | EOS27676 | c_5_hs gi\|6531959\|ref\|gn 1 − 229066 229124 ex 3 6 CDSi 3.01 59 177 | | | 1.3 |
| | | | | CH.05_hs gi\|6531959 | |
| 335166 | EOS35097 | CH22_2502FG_502_10_LINK_EM:AC005500.GENSCAN.396-25 | | | 1.3 |
| | | | | CH22_FGENES.502_10 | |
| 324497 | EOS24428 | AW152624 | Hs.136340 | ESTs | 1.3 |
| 338374 | EOS38305 | CH22_7017FG_LINK_EM:AC005500.GENSCAN.327-1 | | | 1.3 |
| | | | | CH22_EM:AC005500.GENSCAN.327-1 | |
| 313601 | EOS13532 | R32458 | Hs.257711 | ESTs | 1.3 |
| 321415 | EOS21346 | AI377596 | Hs.3337 | transmembrane 4 superfamily member 1 | 1.3 |
| 305309 | EOS05240 | AA699717 | | EST singleton (not in UniGene) with exon hit | 1.3 |
| 330447 | EOS30378 | HG3548-HT3744 | | Pre-Mrna Splicing Factor Sf2p33, Alt. Splice Form 1 | 1.3 |
| 308578 | EOS08509 | AI708573 | | EST singleton (not in UniGene) with exon hit | 1.3 |
| 315344 | EOS15275 | AW292176 | Hs.245834 | ESTs | 1.3 |
| 330503 | EOS30434 | M55024 | | Human cell surface glycoprotein P3.58 mRNA, partial cds | 1.3 |
| 308227 | EOS08158 | AI559126 | Hs.195188 | glyceraldehyde-3-phosphate dehydrogenase | 1.3 |
| 332222 | EOS32153 | N28271 | Hs.176618 | ESTs | 1.3 |
| 323961 | EOS23892 | AL044428 | Hs.207345 | ESTs | 1.3 |
| 314530 | EOS14461 | AI052358 | Hs.131741 | ESTs | 1.3 |
| 320503 | EOS20434 | NM_005897 | | EST cluster (not in UniGene) | 1.3 |
| 306820 | EOS06751 | AI074408 | | EST singleton (not in UniGene) with exon hit | 1.3 |
| 304165 | EOS04096 | H73265 | | EST singleton (not in UniGene) with exon hit | 1.3 |
| 324302 | EOS24233 | AA543008 | Hs.136806 | ESTs; Weakly similar to !!!! ALU SUBFAMILY J WARNING ENTRY !!!! [H. sapiens] | 1.3 |
| 319128 | EOS19059 | AA393820 | | EST cluster (not in UniGene) | 1.3 |
| 317092 | EOS17023 | AI286162 | Hs.125657 | ESTs | 1.3 |
| 304998 | EOS04929 | AA621203 | | EST singleton (not in UniGene) with exon hit | 1.3 |
| 331433 | EOS31364 | H68097 | Hs.161023 | EST | 1.3 |
| 333348 | EOS33279 | CH22_594FG_140_2_LINK_EM:AC005500.GENSCAN.20-2 | | | 1.3 |
| | | | | CH22_FGENES.140_2 | |
| 333619 | EOS33550 | CH22_880FG_219_3_LINK_EM:AC005500.GENSCAN.87-2 | | | 1.3 |
| | | | | CH22.FGENES.219_3 | |
| 335903 | EOS35834 | CH22_3280FG_635_11_LINK_EM:AC005500.GENSCAN.525-14 | | | 1.3 |
| | | | | CH22_FGENES.635_11 | |
| 326219 | EOS26150 | c17.hs gi\|5867226\|ref\|gn 11 − 264008 264274 ex 3 5 CDSi 5.74 267 2847 | | | 1.3 |
| | | | | CH.17_hs gi\|5867226 | |
| 324456 | EOS24387 | AW500954 | | EST cluster (not in UniGene) | 1.3 |
| 316405 | EOS16336 | AA757900 | Hs.202624 | ESTs | 1.3 |
| 314361 | EOS14292 | AL038765 | Hs.161304 | ESTs | 1.3 |
| 328546 | EOS28477 | c_7_hs gi\|5868487\|ref\|gn 1 − 17547 17722 ex 2 3 CDSi 9.96 176 3284 | | | 1.3 |
| | | | | CH.07_hs gi\|5868487 | |
| 335871 | EOS35802 | CH22_3246FG_629_19_LINK_EM:AC005500.GENSCAN.519-18 | | | 1.3 |
| | | | | CH22_FGENES.629_19 | |
| 303735 | EOS03666 | AA707750 | Hs.202616 | ESTs; Weakly similar to cis-Golgi matrix protein GM130 [R. norvegicus] | 1.3 |
| 324048 | EOS23979 | AA378739 | | EST cluster (not in UniGene) | 1.3 |
| 326720 | EOS26651 | c20_hs gi\|6552456\|ref\|gn 1 + 84525 84677 ex 5 7 CDSi 11.78 153 1031 | | | 1.3 |
| | | | | CH.20_hs gi\|6552456 | |
| 322309 | EOS22240 | AF086372 | | EST cluster (not in UniGene) | 1.3 |
| 322136 | EOS22067 | AF075083 | | EST cluster (not in UniGene) | 1.3 |
| 313460 | EOS13391 | AW028655 | Hs.136033 | ESTs | 1.3 |
| 306275 | EOS06206 | AA936312 | | EST singleton (not in UniGene) with exon hit | 1.3 |
| 321974 | EOS21905 | N76794 | | EST cluster (not in UniGene) | 1.3 |
| 327600 | EOS27531 | c_3_hs gi\|6004462\|ref\|gn 1 − 2621 2862 ex 1 4 CDSl - 4.01 242 1407 | | | 1.3 |
| | | | | CH.03_hs gi\|6004462 | |
| 329086 | EOS29017 | c_x_hs gi\|5868604\|ref\|gn 1 − 35489 35588 ex 2 9 CDSi 2.55 100 719 | | | 1.3 |
| | | | | CH.X_hs gi\|5868604 | |
| 336919 | EOS36850 | CH22_4690FG_{—} | | CH22_FGENES.346-6 | 1.3 |
| | | 346_6_{—} | | | |
| 302767 | EOS02698 | H94900 | Hs.17882 | ESTs | 1.3 |
| 334786 | EOS34717 | CH22_2098FG_432_11_LINK_EM:AC005500.GENSCAN.293-14 | | | 1.3 |
| | | | | CH22_FGENES.432_11 | |
| 302472 | EOS02403 | AA317451 | Hs.241451 | SWI/SNF related; matrix associated; actin dependent regulator | 1.3 |
| | | | | of chromatin; subfamily e; member 1 | |
| 333033 | EOS32964 | CH22_259FG_68_8_LINK_EM:AC000097.GENSCAN.40-8 | | | 1.3 |
| | | | | CH22_FGENES.68_8 | |
| 330493 | EOS30424 | M27826 | Hs.238380 | Human endogenous retroviral protease mRNA; complete cds | 1.3 |
| 330506 | EOS30437 | M61906 | Hs.6241 | phosphoinositide-3-kinase; regulatory subunit; polypeptide 1 (p85 alpha) | 1.3 |
| 313932 | EOS13863 | AI147601 | Hs.154087 | ESTs | 1.3 |
| 314394 | EOS14325 | AI380563 | Hs.130816 | ESTs | 1.3 |
| 323033 | EOS22964 | AI744284 | Hs.221727 | ESTs | 1.3 |
| 326431 | EOS26362 | c19_hs gi\|5867371\|ref\|gn 1 + 15855 15971 ex 4 6 CDSi 7.79 117 1108 | | | 1.3 |
| | | | | CH.19_hs gi\|5867371 | |
| 335547 | EOS35478 | CH22_2902FG_576_8_LINK_EM:AC005500.GENSCAN.467-8 | | | 1.3 |
| | | | | CH22_FGENES.576_8 | |
| 300548 | EOS00479 | AI026836 | Hs.114689 | ESTs | 1.3 |
| 316504 | EOS16435 | AW135854 | Hs.132458 | ESTs | 1.3 |
| 335756 | EOS35687 | CH22_3123FG_604_5_LINK_EM:AC005500.GENSCAN.493-10 | | | 1.3 |
| | | | | CH22_FGENES.604_5 | |
| 301209 | EOS01140 | AI809912 | Hs.159354 | ESTs | 1.3 |
| 306610 | EOS06541 | AI000635 | | EST singleton (not in UniGene) with exon hit | 1.3 |
| 314439 | EOS14370 | AI539443 | Hs.137447 | ESTs | 1.3 |
| 315396 | EOS15327 | AW296107 | Hs.152686 | ESTs | 1.3 |
| 335914 | EOS35845 | CH22_3291FG_636_10_LINK_EM:AC005500.GENSCAN.526-10 | | | 1.3 |
| | | | | CH22_FGENES.636_10 | |
| 333734 | EOS33665 | CH22_1000FG_260_2_LINK_EM:AC005500.GENSCAN.119-7 | | | 1.3 |
| | | | | CH22_FGENES.260_2 | |
| 312370 | EOS12301 | AA744692 | Hs.166539 | ESTs | 1.3 |
| 304636 | EOS04567 | AA524031 | | EST singleton (not in UniGene) with exon hit | 1.3 |
| 323166 | EOS23097 | AA291001 | | EST cluster (not in UniGene) | 1.3 |
| 338702 | EOS38633 | CH22_7482FG_LINK_EM:AC005500.GENSCAN.480-1 | | | 1.3 |
| | | | | CH22_EM:AC005500.GENSCAN.480-1 | |
| 322331 | EOS22262 | AF086467 | | EST cluster (not in UniGene) | 1.3 |
| 318706 | EOS18637 | AI383593 | Hs.159148 | ESTs | 1.3 |
| 331186 | EOS31117 | T41159 | Hs.8418 | ESTs | 1.3 |
| 334764 | EOS34695 | CH22_2076FG_428_13_LINK_EM:AC005500.GENSCAN.289-13 | | | 1.3 |
| | | | | CH22_FGENES.428_13 | |
| 327565 | EOS27496 | c_3_hs gi\|5867811\|ref\|gn 1 + 32516 32778 ex 2 3 CDSi 0.20 263 368 | | | 1.3 |
| | | | | CH.03_hs gi\|5867811 | |
| 335524 | EOS35455 | CH22_2879FG_572_4_LINK_EM:AC005500.GENSCAN.461-4 | | | 1.3 |
| | | | | CH22_FGENES.572_4 | |
| 308050 | EOS07981 | AI460004 | | EST singleton (not in UniGene) with exon hit | 1.3 |
| 334172 | EOS34103 | CH22_1452FG_349_5_LINK_EM:AC005500.GENSCAN.208-6 | | | 1.3 |
| | | | | CH22_FGENES.349_5 | |
| 315674 | EOS15605 | AA651923 | Hs.191850 | ESTs | 1.3 |
| 334876 | EOS34807 | CH22_2190FG_450_6_LINK_EM:AC005500.GENSCAN.339-6 | | | 1.3 |
| | | | | CH22_FGENES.450_6 | |
| 315606 | EOS15537 | AW298724 | Hs.202639 | ESTs | 1.3 |
| 338779 | EOS38710 | CH22_7610FG_LINK_EM:AC005500.GENSCAN.526-15 | | | 1.3 |
| | | | | CH22_EM:AC005500.GENSCAN.526-15 | |
| 333511 | EOS33442 | CH22_766FG_171_5_LINK_EM:AC005500.GENSCAN.51-5 | | | 1.3 |
| | | | | CH22_FGENES.171_5 | |
| 329254 | EOS29185 | c_x_hs gi\|5868733\|ref\|gn 1 + 4133 4214 ex 1 2 CDSi-0.36 82 2833 | | | 1.3 |
| | | | | CH.X_hs gi\|5868733 | |
| 319510 | EOS19441 | W88633 | Hs.254562 | ESTs | 1.3 |
| 339418 | EOS39349 | CH22_8411FG_LINK_DJ579N16.GENSCAN.11-4 | | | 1.3 |
| | | | | CH22_DJ579N16.GENSCAN.11-4 | |
| 321012 | EOS20943 | AA737314 | | EST cluster (not in UniGene) | 1.3 |
| 333217 | EOS33148 | CH22_454FG_104_9_LINK_EM:AC000097.GENSCAN.108-8 | | | 1.3 |
| | | | | CH22_FGENES.104_9 | |
| 338561 | EOS38492 | CH22_7294FG_LINK_EM:AC005500.GENSCAN.421-5 | | | 1.3 |
| | | | | CH22_EM:AC005500.GENSCAN.421-5 | |
| 335742 | EOS35673 | CH22_3105FG_601_13_LINK_EM:AC005500.GENSCAN.491-14 | | | 1.3 |
| | | | | CH22_FGENES.601_13 | |
| 334993 | EOS34924 | CH22_2314FG_469_14_LINK_EM:AC005500.GENSCAN.365-16 | | | 1.3 |
| | | | | CH22_FGENES.469_14 | |
| 323430 | EOS23361 | AW062479 | | EST cluster (not in UniGene) | 1.3 |
| 306069 | EOS06000 | AA906983 | | EST singleton (not in UniGene) with exon hit | 1.3 |
| 331681 | EOS31612 | W85712 | Hs.119571 | collagen; type III; alpha 1 (Ehlers-Danlos syndrome type IV; autosomal dominant) | 1.3 |
| 337986 | EOS37917 | CH22_6441FG_LINK_EM:AC005500.GENSCAN.110-7 | | | 1.3 |
| | | | | CH22_EM:AC005500.GENSCAN.110-7 | |
| 313204 | EOS13135 | AI800518 | Hs.118158 | ESTs | 1.3 |
| 323189 | EOS23120 | AL121194 | Hs.120589 | ESTs | 1.3 |
| 318171 | EOS18102 | AA381202 | | EST cluster (not in UniGene) | 1.3 |
| 307156 | EOS07087 | AI186762 | | EST singleton (not in UniGene) with exon hit | 1.3 |
| 332713 | EOS32644 | AA349792 | Hs.78489 | mutY (E. coli) homolog | 1.3 |
| 312828 | EOS12759 | AI865455 | Hs.211818 | ESTs; Moderately similar to !!!! ALU SUBFAMILY J WARNING | 1.3 |
| | | | | ENTRY !!!! [H. sapiens] | |
| 301127 | EOS01058 | AA758109 | Hs.121072 | ESTs | 1.3 |
| 311260 | EOS11191 | AI672509 | Hs.196582 | ESTs | 1.3 |
| 338364 | EOS38295 | CH22_7007FG_LINK_EM:AC005500.GENSCAN.323-7 | | | 1.3 |
| | | | | CH22_EM:AC005500.GENSCAN.323-7 | |
| 337904 | EOS37835 | CH22_6318FG_LINK_EM:AC005500.GENSCAN.56-17 | | | 1.3 |
| | | | | CH22_EM:AC005500.GENSCAN.56-17 | |
| 329347 | EOS29278 | c_x_hs gi\|6456785\|ref\|gn 1 + 18433 18897 ex 4 4 CDSl 43.39 465 3718 | | | 1.3 |
| | | | | CH.X_hs gi\|6456785 | |
| 313329 | EOS13260 | AW293704 | Hs.122658 | ESTs | 1.3 |
| 314367 | EOS14298 | AA535749 | | EST cluster (not in UniGene) | 1.3 |
| 317098 | EOS17029 | AI123513 | Hs.125456 | ESTs | 1.3 |
| 306462 | EOS06393 | AA983397 | | EST singleton (not in UniGene) with exon hit | 1.3 |
| 301254 | EOS01185 | AI049624 | | EST cluster (not in UniGene) with exon hit | 1.3 |
| 335504 | EOS35435 | CH22_2856FG_571_15_LINK_EM:AC005500.GENSCAN.460-34 | | | 1.3 |
| | | | | CH22_FGENES.571_15 | |
| 334270 | EOS34201 | CH22_1559FG_368_2_LINK_EM:AC005500.GENSCAN.228-3 | | | 1.3 |
| | | | | CH22_FGENES.368_2 | |
| 334324 | EOS34255 | CH22_1616FG_375_1_LINK_EM:AC005500.GENSCAN.235-1 | | | 1.3 |
| | | | | CH22_FGENES.375_1 | |
| 304254 | EOS04185 | AA046273 | Hs.111334 | ferntin; light polypeptide | 1.3 |
| 305731 | EOS05662 | AA829363 | | EST singleton (not in UniGene) with exon hit | 1.3 |
| 323284 | EOS23215 | AA279381 | Hs.190010 | ESTs | 1.3 |
| 322007 | EOS21938 | AW410646 | Hs.165739 | ESTs | 1.3 |
| 334537 | EOS34468 | CH22_1839FG_403_2_LINK_EM:AC005500.GENSCAN.268-2 | | | 1.3 |
| | | | | CH22_FGENES.403_2 | |
| 302360 | EOS02291 | AJ010901 | Hs.198267 | mucin 4; tracheobronchial | 1.3 |
| 311641 | EOS11572 | AI948829 | Hs.213786 | ESTs | 1.3 |
| 324643 | EOS24574 | AI436356 | Hs.130729 | ESTs | 1.3 |
| 327554 | EOS27485 | c_3_hs gi\|5867801\|ref\|gn 2 − 23092 23191 ex 2 6 CDSi 10.44 100 107 | | | 1.3 |
| | | | | CH.03_hs gi\|5867801 | |
| 312165 | EOS12096 | AW292139 | Hs.115789 | ESTs | 1.3 |
| 304679 | EOS04610 | AA548741 | | EST singleton (not in UniGene) with exon hit | 1.3 |
| 319564 | EOS19495 | AA026777 | Hs.169732 | ESTs | 1.3 |
| 310860 | EOS10791 | AW015920 | Hs.161359 | ESTs | 1.3 |
| 337161 | EOS37092 | CH22_5180FG_{—} | | CH22_FGENES.561-3 | 1.3 |
| | | 561_3_{—} | | | |
| 311155 | EOS11086 | AI634410 | Hs.197608 | EST | 1.3 |
| 336846 | EOS36777 | CH22_4540FG_{—} | | CH22_FGENES.263-5 | 1.3 |
| | | 263_5_{—} | | | |
| 310985 | EOS10916 | T51842 | | EST cluster (not in UniGene) | 1.3 |
| 329499 | EOS29430 | c10_p2 gi\|3983518\|gb\|A gn 5 + 33463 33789 ex 1 1 CDSo 34.50 327 97 | | | 1.3 |
| | | | | CH.10_p2 gi\|3983518 | |
| 334924 | EOS34855 | CH22_2244FG_459_2_LINK_EM:AC005500.GENSCAN.351-2 | | | 1.3 |
| | | | | CH22_FGENES.459_2 | |
| 330861 | EOS30792 | AA084064 | Hs.185747 | ESTs | 1.3 |
| 324658 | EOS24589 | AI694767 | Hs.129179 | ESTs | 1.3 |
| 323362 | EOS23293 | AL135067 | Hs.117182 | ESTs | 1.3 |
| 330468 | EOS30399 | L10343 | Hs.112341 | protease inhibitor 3; skin-derived (SKALP) | 1.3 |
| 314198 | EOS14129 | AA897581 | Hs.128773 | ESTs | 1.3 |
| 339436 | EOS39367 | CH22_8431FG_LINK_DJ579N16.GENSCAN. 19-1 | | | 1.3 |
| | | | | CH22_DJ579N16.GENSCAN. 19-1 | |

| | | | | | |
|---|---|---|---|---|---|
| 312483 | EOS12414 | AI417526 | Hs.184636 | ESTs | 1.3 |
| 321505 | EOS21436 | H73183 | Hs.129885 | ESTs | 1.3 |
| 332254 | EOS32185 | N64702 | Hs.194140 | ESTs | 1.3 |

| 328253 | EOS28184 | c_6_hs gi\|6381894\|ref\|gn 1 − 4411 4509 ex 1 5 CDSl 4.20 99 4561 | | | 1.3 |
|---|---|---|---|---|---|
| | | | | CH.06_hs gi\|6381894 | |
| 332357 | EOS32288 | W73417 | Hs.103183 | EST | 1.3 |
| 329017 | EOS28948 | c_x_hs gi\|6682532\|ref\|gn 7 − 255591 255672 ex 3 3 CDSi 12.94 82 22 | | | 1.3 |
| | | | | CH.X_hs gi\|6682532 | |
| 337504 | EOS37435 | CH22_5739FG_{—} | | CH22_FGENES.803-2 | 1.3 |
| | | 803_2_{—} | | | |
| 316625 | EOS16556 | AA780307 | Hs.122156 | ESTs | 1.3 |
| 335389 | EOS35320 | CH22_2739FG_545_1_LINK_EM:AC005500.GENSCAN.436-1 | | | 1.3 |
| | | | | CH22_FGENES.545_1 | |
| 310017 | EOS09948 | AI188739 | Hs.148488 | ESTs | 1.3 |
| 314354 | EOS14285 | AL037984 | Hs.208982 | ESTs; Weakly similar to !!!! ALU SUBFAMILY J WARNING ENTRY !!!! [H. sapiens] | 1.3 |
| 324641 | EOS24572 | AI732515 | Hs.189218 | ESTs | 1.3 |
| 335207 | EOS35138 | CH22_2546FG_510_4_LINK_EM:AC005500.GENSCAN.402-3 | | | 1.3 |
| | | | | CH22_FGENES.510_4 | |
| 333673 | EOS33604 | CH22_934FG_246_5_LINK_EM:AC005500.GENSCAN.101-3 | | | 1.3 |
| | | | | CH22_FGENES.246_5 | |
| 334370 | EOS34301 | CH22_1664FG_378_18_LINK_EM:AC005500.GENSCAN.240-1 | | | 1.3 |
| | | | | CH22_FGENES.378_18 | |
| 328690 | EOS28621 | c_7_hs gi\|6588001\|ref\|gn 7 − 571207 571274 ex 1 3 CDSl 3.34 68 4325 | | | 1.3 |
| | | | | CH.07_hs gi\|6588001 | |
| 323208 | EOS23139 | AA203415 | Hs.136200 | ESTs | 1.3 |
| 307010 | EOS06941 | AI140014 | | EST singleton (not in UniGene) with exon hit | 1.3 |
| 316563 | EOS16494 | AI587083 | Hs.200558 | ESTs; Weakly similar to !!!! ALU SUBFAMILY SP WARNING ENTRY !!!! [H. sapiens] | 1.3 |
| 312219 | EOS12150 | H73505 | Hs.117874 | ESTs | 1.3 |
| 319884 | EOS19815 | T73234 | | EST cluster (not in UniGene) | 1.3 |
| 334720 | EOS34651 | CH22_2030FG_421_31_LINK_EM:AC005500.GENSCAN.282-31 | | | 1.3 |
| | | | | CH22_FGENES.421_31 | |
| 335836 | EOS35767 | CH22_3210FG_621_3_LINK_EM:AC005500.GENSCAN.513-3 | | | 1.3 |
| | | | | CH22_FGENES.621_3 | |
| 305448 | EOS05379 | AA737894 | Hs.29797 | ribosomal protein L10 | 1.3 |
| 314885 | EOS14816 | AI049878 | Hs.133032 | ESTs | 1.3 |
| 320130 | EOS20061 | AI820675 | Hs.203804 | ESTs | 1.3 |
| 310567 | EOS10498 | AI691065 | Hs.155780 | ESTs | 1.3 |
| 323898 | EOS23829 | AA347566 | | EST cluster (not in UniGene) | 1.3 |
| 336132 | EOS36063 | CH22_3522FG_703_2_LINK_DA59H18.GENSCAN.9-2 | | | 1.3 |
| | | | | CH22_FGENES.703_2 | |
| 337958 | EOS37889 | CH22_6403FG_LINK_EM:AC005500.GENSCAN.98-6 | | | 1.3 |
| | | | | CH22_EM:AC005500.GENSCAN.98-6 | |
| 305630 | EOS05561 | AA804508 | | EST singleton (not in UniGene) with exon hit | 1.3 |
| 334916 | EOS34847 | CH22_2235FG_457_7_LINK_EM:AC005500.GENSCAN.347-1 | | | 1.3 |
| | | | | CH22_FGENES.457_7 | |
| 333542 | EOS33473 | CH22_799FG_178_4_LINK_EM:AC005500.GENSCAN.59-4 | | | 1.3 |
| | | | | CH22_FGENES.178_4 | |
| 331151 | EOS31082 | R82331 | Hs.164599 | ESTs | 1.3 |
| 315095 | EOS15026 | AA831815 | Hs.243788 | ESTs | 1.3 |
| 331593 | EOS31524 | N72150 | Hs.50193 | EST | 1.3 |
| 323767 | EOS23698 | AI807408 | Hs.166368 | ESTs | 1.3 |
| 334561 | EOS34492 | CH22_1865FG_405_1_LINK_EM:AC005500.GENSCAN.270-5 | | | 1.3 |
| | | | | CH22_FGENES.405_1 | |
| 308191 | EOS08122 | AI538878 | | EST singleton (not in UniGene) with exon hit | 1.3 |
| 319571 | EOS19502 | N91399 | Hs.220826 | ESTs | 1.3 |
| 316200 | EOS16131 | AI914535 | Hs.221377 | ESTs | 1.3 |
| 305996 | EOS05927 | AA889338 | Hs.163356 | EST | 1.2 |
| 318055 | EOS17986 | AI249193 | Hs.145945 | ESTs | 1.2 |
| 315570 | EOS15501 | AI860360 | Hs.160316 | ESTs | 1.2 |
| 320792 | EOS20723 | AW236504 | Hs.247020 | ESTs | 1.2 |
| 331649 | EOS31580 | W20364 | Hs.55412 | ESTs; Weakly similar to c29 [M. musculus] | 1.2 |
| 303839 | EOS03770 | Z45939 | | EST cluster (not in UniGene) with exon hit | 1.2 |
| 324399 | EOS24330 | AA814768 | Hs.21396 | ESTs | 1.2 |
| 317172 | EOS17103 | AI741232 | Hs.206744 | ESTs | 1.2 |
| 312452 | EOS12383 | AI692643 | Hs.172749 | ESTs | 1.2 |
| 325482 | EOS25413 | c12_hs gi\|5866957\|ref\|gn 3 + 47957 48078 ex 5 7 CDSi 10.25 122 1896 | | | 1.2 |
| | | | | CH.12_hs gi\|5866957 | |
| 311395 | EOS11326 | R23313 | | EST cluster (not in UniGene) | 1.2 |
| 336124 | EOS36055 | CH22_3513FG_701_9_LINK_DA59H18.GENSCAN.8-9 | | | 1.2 |
| | | | | CH22_FGENES.701_9 | |
| 320082 | EOS20013 | AA487678 | Hs.189738 | ESTs | 1.2 |
| 312168 | EOS12099 | T92251 | Hs.198882 | ESTs | 1.2 |
| 338000 | EOS37931 | CH22_6472FG_LINK_EM:AC005500.GENSCAN.119-5 | | | 1.2 |
| | | | | CH22_EM:AC005500.GENSCAN.119-5 | |
| 338852 | EOS38783 | CH22_7705FG_LINK_DJ246D7.GENSCAN.12-1 | | | 1.2 |
| | | | | CH22_DJ246D7.GENSCAN.12-1 | |
| 312090 | EOS12021 | N57692 | Hs.118064 | ESTs | 1.2 |
| 316480 | EOS16411 | AI749921 | Hs.205377 | ESTs | 1.2 |
| 333259 | EOS33190 | CH22_500FG_118_7_LINK_EM:AC005500.GENSCAN.2-7 | | | 1.2 |
| | | | | CH22_FGENES.118_7 | |
| 335211 | EOS35142 | CH22_2550FG_511_2_LINK_EM:AC005500.GENSCAN.403-2 | | | 1.2 |
| | | | | CH22_FGENES.511_2 | |
| 321950 | EOS21881 | AA594780 | Hs.172318 | ESTs | 1.2 |
| 337937 | EOS37868 | CH22_6370FG_LINK_EM:AC005500.GENSCAN.86-1 | | | 1.2 |
| | | | | CH22_EM:AC005500.GENSCAN.86-1 | |
| 316576 | EOS16507 | AI732114 | Hs.193046 | ESTs; Weakly similar to !!!! ALU SUBFAMILY J WARNING ENTRY !!!! [H. sapiens] | 1.2 |
| 322770 | EOS22701 | AA045796 | Hs.159971 | SWI/SNF related; matrix associated; actin dependent regulator | 1.2 |
| | | | | of chromatin; subfamily b; member 1 | |
| 329369 | EOS29300 | c_x_hs gi\|5868842\|ref\|gn 1 − 121148 121516 ex 3 4 CDSi 8.50 369 3910 | | | 1.2 |
| | | | | CH.X_hs gi\|5868842 | |
| 304183 | EOS04114 | H91161 | | EST singleton (not in UniGene) with exon hit | 1.2 |
| 339370 | EOS39301 | CH22_8343FG_LINK_BA232E17.GENSCAN.1-12 | | | 1.2 |
| | | | | CH22_BA232E17.GENSCAN.1-12 | |
| 303941 | EOS03872 | AW473878 | Hs.156110 | Immunoglobulin kappa variable 1D-8 | 1.2 |
| 302245 | EOS02176 | H18835 | | EST cluster (not in UniGene) with exon hit | 1.2 |
| 335255 | EOS35186 | CH22_2597FG_517_2_LINK_EM:AC005500.GENSCAN.411-2 | | | 1.2 |
| | | | | CH22_FGENES.517_2 | |
| 316610 | EOS16541 | AW087973 | Hs.126731 | ESTs | 1.2 |
| 314915 | EOS14846 | AA573072 | Hs.187748 | ESTs; Weakly similar to !!!! ALU SUBFAMILY J WARNING ENTRY !!!! [H. sapiens] | 1.2 |
| 315426 | EOS15357 | AI391486 | Hs.128171 | ESTs | 1.2 |
| 334003 | EOS33934 | CH22_1281FG_310_28_LINK_EM:AC005500.GENSCAN.167-27 | | | 1.2 |
| | | | | CH22_FGENES.310_28 | |
| 304350 | EOS04281 | AA186871 | | EST singleton (not in UniGene) with exon hit | 1.2 |
| 325173 | EOS25104 | AI133215 | Hs.144662 | ESTs; Moderately similar to !!!! ALU SUBFAMILY J WARNING | 1.2 |
| | | | | ENTRY !!!! [H. sapiens] | |
| 312313 | EOS12244 | AW293341 | Hs.122505 | ESTs | 1.2 |
| 333366 | EOS33297 | CH22_612FG_142_3_LINK_EM:AC005500.GENSCAN.22-6 | | | 1.2 |
| | | | | CH22_FGENES.142_3 | |
| 334970 | EOS34901 | CH22_2291FG_466_3_LINK_EM:AC005500.GENSCAN.361-2 | | | 1.2 |
| | | | | CH22_FGENES.466_3 | |
| 338668 | EOS38599 | CH22_7441FG_LINK_EM:AC005500.GENSCAN.465-1 | | | 1.2 |
| | | | | CH22_EM:AC005500.GENSCAN.465-1 | |
| 336502 | EOS36433 | CH22_3926FG_833_8_LINK_DJ579N16.GENSCAN.5-9 | | | 1.2 |
| | | | | CH22_FGENES.833_8 | |
| 309438 | EOS09369 | AW102802 | Hs.225787 | ESTs; Moderately similar to hypothetical protein [H. sapiens] | 1.2 |
| 336194 | EOS36125 | CH22_3591FG_717_20_LINK_DA59H18.GENSCAN.20-19 | | | 1.2 |
| | | | | CH22_FGENES.717_20 | |
| 336678 | EOS36609 | CH22_4156FG_43_6_{—} | | CH22_FGENES.43-6 | 1.2 |
| 321401 | EOS21332 | W90406 | Hs.35962 | ESTs | 1.2 |
| 306026 | EOS05957 | AA902309 | | EST singleton (not in UniGene) with exon hit | 1.2 |
| 336434 | EOS36365 | CH22_3854FG_826_1_LINK_BA232E17.GENSCAN.8-1 | | | 1.2 |
| | | | | CH22_FGENES.826_1 | |
| 315257 | EOS15188 | AW157431 | Hs.248941 | ESTs | 1.2 |
| 328349 | EOS28280 | c_7_hs gi\|5868383\|ref\|gn 7 − 260704 260804 ex 2 9 CDSi 4.37 101 621 | | | 1.2 |
| | | | | CH.07_hs gi\|5868383 | |
| 326112 | EOS26043 | c17_hs gi\|5867192\|ref\|gn 1 + 2151 2725 ex 1 1 CDSl 54.87 575 1272 | | | 1.2 |
| | | | | CH.17_hs gi\|5867192 | |
| 333995 | EOS33926 | CH22_1272FG_310_19_LINK_EM:AC005500.GENSCAN.167-18 | | | 1.2 |
| | | | | CH22_FGENES.310_19 | |
| 323683 | EOS23614 | AI380045 | Hs.225033 | ESTs | 1.2 |
| 330143 | EOS30074 | c21_p2 gi\|4210430\|emb\|gn 3 + 184737 184848 ex 4 4 CDSl 1.71 112 111 | | | 1.2 |
| | | | | CH.21_p2 gi\|4210430 | |
| 329789 | EOS29720 | c14_p2 gi\|6469354\|emb\|gn 2 − 118977 119036 ex 1 3 CDSl 1.19 60 1517 | | | 1.2 |
| | | | | CH.14_p2 gi\|6469354 | |
| 324397 | EOS24328 | AA307836 | Hs.118758 | ESTs; Weakly similar to RLF [H. sapiens] | 1.2 |
| 308729 | EOS08660 | AI799766 | Hs.208627 | EST | 1.2 |
| 323939 | EOS23870 | AW499632 | Hs.115696 | ESTs | 1.2 |
| 333444 | EOS33375 | CH22_694FG_153_1_LINK_EM:AC005500.GENSCAN.34-1 | | | 1.2 |
| | | | | CH22_FGENES.153_1 | |
| 306302 | EOS06233 | AA937901 | | EST singleton (not in UniGene) with exon hit | 1.2 |
| 313693 | EOS13624 | AW469180 | Hs.170651 | ESTs | 1.2 |
| 316652 | EOS16583 | AA789249 | | EST cluster (not in UniGene) | 1.2 |
| 332325 | EOS32256 | T79428 | Hs.191264 | ESTs | 1.2 |
| 336235 | EOS36166 | CH22_3633FG_740_2_LINK_DA59H18.GENSCAN.44-2 | | | 1.2 |
| | | | | CH22_FGENES.740_2 | |
| 319436 | EOS19367 | R02750 | | EST cluster (not in UniGene) | 1.2 |
| 312335 | EOS12266 | AW043620 | Hs.236993 | ESTs | 1.2 |
| 322109 | EOS22040 | AI884327 | Hs.244737 | ESTs | 1.2 |
| 328466 | EOS28397 | c_7_hs gi\|5868434\|ref\|gn 1 − 15643 15900 ex 1 2 CDSl 2.36 258 1608 | | | 1.2 |
| | | | | CH.07_hs gi\|5868434 | |
| 323244 | EOS23175 | T70731 | | EST cluster (not in UniGene) | 1.2 |
| 312510 | EOS12441 | AA779907 | Hs.117558 | ESTs | 1.2 |
| 314853 | EOS14784 | AA729232 | Hs.153279 | ESTs | 1.2 |
| 336946 | EOS36877 | CH22_4731FG_{—} | | CH22_FGENES.355-2 | 1.2 |
| | | 355_2_{—} | | | |
| 303874 | EOS03805 | AA258921 | | EST cluster (not in UniGene) with exon hit | 1.2 |
| 312658 | EOS12589 | AA730280 | Hs.120936 | ESTs | 1.2 |
| 308354 | EOS08285 | AI611044 | | EST singleton (not in UniGene) with exon hit | 1.2 |
| 310073 | EOS10004 | AI335004 | Hs.148558 | ESTs | 1.2 |
| 324777 | EOS24708 | AA744046 | Hs.133350 | ESTs | 1.2 |
| 300897 | EOS00828 | AI890356 | Hs.127804 | ESTs | 1.2 |
| 308371 | EOS08302 | AI620666 | Hs.242510 | EST | 1.2 |
| 306358 | EOS06289 | AA961821 | | EST singleton (not in UniGene) with exon hit | 1.2 |
| 312295 | EOS12226 | AA578233 | Hs.173863 | ESTs | 1.2 |
| 319792 | EOS19723 | R20317 | Hs.22968 | ESTs | 1.2 |
| 338546 | EOS38477 | CH22_7267FG_LINK_EM:AC005500.GENSCAN.410-1 | | | 1.2 |
| | | | | CH22_EM:AC005500.GENSCAN.410-1 | |
| 314546 | EOS14477 | AW007211 | Hs.186672 | ESTs | 1.2 |
| 338494 | EOS38425 | CH22_7184FG_LINK_EM:AC005500.GENSCAN.385-5 | | | 1.2 |
| | | | | CH22_EM:AC005500.GENSCAN.385-5 | |
| 331131 | EOS31062 | R54797 | Hs.26238 | EST; Weakly similar to reverse transcriptase homolog [H. sapiens] | 1.2 |
| 309939 | EOS09870 | AW419122 | | EST singleton (not in UniGene) with exon hit | 1.2 |
| 332932 | EOS32863 | CH22_153FG_38_6_LINK_C20H12.GENSCAN.29-6 | | | 1.2 |
| | | | | CH22_FGENES.38_6 | |
| 309653 | EOS09584 | AW196800 | Hs.180842 | ribosomal protein L13 | 1.2 |
| 318647 | EOS18578 | AI526152 | | EST cluster (not in UniGene) | 1.2 |
| 304044 | EOS03975 | T52479 | Hs.252259 | ribosomal protein S3 | 1.2 |
| 330307 | EOS30238 | c_7_p2 gi\|4877982\|gb\|A gn 2 + 107384 107559 ex 2 4 CDSi 9.96 176 4 | | | 1.2 |
| | | | | CH.07_p2 gi\|4877982 | |
| 314499 | EOS14430 | AL044570 | Hs.147975 | ESTs | 1.2 |
| 338053 | EOS37984 | CH22_6552FG_LINK_EM:AC005500.GENSCAN.158-1 | | | 1.2 |
| | | | | CH22_EM:AC005500.GENSCAN.158-1 | |
| 332991 | EOS32922 | CH22_215FG_56_4_LINK_EM:AC000097.GENSCAN.17-4 | | | 1.2 |
| | | | | CH22_FGENES.56_4 | |
| 306308 | EOS06239 | AA946870 | | EST singleton (not in UniGene) with exon hit | 1.2 |
| 338120 | EOS38051 | CH22_6655FG_LINK_EM:AC005500.GENSCAN.195-1 | | | 1.2 |
| | | | | CH22_EM:AC005500.GENSCAN.195-1 | |
| 313703 | EOS13634 | AI161293 | Hs.146862 | ESTs; Weakly similar to KIAA0525 protein [H. sapiens] | 1.2 |
| 330563 | EOS30494 | U50553 | Hs.147916 | DEAD/H (Asp-Glu-Ala-Asp/His) box polypeptide 3 | 1.2 |
| 332886 | EOS32817 | CH22_106FG_33_7_LINK_C20H12.GENSCAN.22-9 | | | 1.2 |
| | | | | CH22_FGENES.33_7 | |
| 303844 | EOS03775 | U94362 | Hs.58589 | glycogenin 2 | 1.2 |
| 321755 | EOS21686 | AI215881 | Hs.144042 | ESTs | 1.2 |
| 333532 | EOS33463 | CH22_789FG_175_19_LINK_EM:AC005500.GENSCAN.53-25 | | | 1.2 |
| | | | | CH22_FGENES.175_19 | |
| 332863 | EOS32794 | CH22_81FG_28_3_LINK_C20H12.GENSCAN.18-3 | | | 1.2 |
| | | | | CH22_FGENES.28_3 | |
| 333254 | EOS33185 | CH22_495FG_118_2_LINK_EM:AC005500.GENSCAN.2-2 | | | 1.2 |
| | | | | CH22_FGENES.118_2 | |
| 317459 | EOS17390 | AI367254 | Hs.131248 | ESTs | 1.2 |
| 315353 | EOS15284 | AW452608 | Hs.129817 | ESTs | 1.2 |
| 300732 | EOS00663 | AI369956 | Hs.257891 | ESTs | 1.2 |
| 303502 | EOS03433 | AA488528 | | EST cluster (not in UniGene) with exon hit | 1.2 |
| 333126 | EOS33057 | CH22_355FG_82_3_LINK_EM:AC000097.GENSCAN.66-10 | | | 1.2 |
| | | | | CH22_FGENES.82_3 | |
| 332929 | EOS32860 | CH22_150FG_38_3_LINK_C20H12.GENSCAN.29-3 | | | 1.2 |
| | | | | CH22_FGENES.38_3 | |
| 329502 | EOS29433 | c10_p2 gi\|3983517\|gb\|U gn 1 + 75 338 ex 1 1 CDSo 46.82 264 100 | | | 1.2 |
| | | | | CH.10_p2 gi\|3983517 | |
| 333408 | EOS33339 | CH22_657FG_145_6_LINK_EM:AC005500.GENSCAN.26-6 | | | 1.2 |
| | | | | CH22_FGENES.145_6 | |
| 315472 | EOS15403 | AA828850 | Hs.165469 | ESTs | 1.2 |
| 328290 | EOS28221 | c_7_hs gi\|5868363\|ref\|gn 2 − 127366 127496 ex 1 5 CDSl 5.24 131 289 | | | 1.2 |
| | | | | CH.07_hs gi\|5868363 | |
| 328662 | EOS28593 | c_7_hs gi\|6004473\|ref\|gn 22 + 1184773 1184855 ex 7 8 CDSi 12.72 83 3916 | | | 1.2 |
| | | | | CH.07_hs gi\|6004473 | |
| 319808 | EOS19739 | T58960 | | EST cluster (not in UniGene) | 1.2 |
| 303929 | EOS03860 | AW470753 | | EST singleton (not in UniGene) with exon hit | 1.2 |
| 315712 | EOS15643 | AI950133 | Hs.120882 | ESTs; Moderately similar to !!!! ALU SUBFAMILY J WARNING | 1.2 |
| | | | | ENTRY !!!! [H. sapiens] | |
| 307391 | EOS07322 | AI225058 | | EST singleton (not in UniGene) with exon hit | 1.2 |
| 335499 | EOS35430 | CH22_2851FG_571_8_LINK_EM:AC005500.GENSCAN.460-26 | | | 1.2 |
| | | | | CH22_FGENES.571_8 | |
| 303792 | EOS03723 | C75094 | Hs.199839 | ESTs; Highly similar to NG22 [H. sapiens] | 1.2 |
| 327287 | EOS27218 | c_1_hs gi\|5867479\|ref\|gn 1 − 62838 63024 ex 4 5 CDSi 11.66 187 1628 | | | 1.2 |
| | | | | CH.01_hs gi\|5867479 | |
| 317713 | EOS17644 | AI733306 | Hs.128071 | ESTs | 1.2 |
| 330137 | EOS30068 | c21_p2 gi\|4210430\|emb\|gn 1 − 21220 21377 ex 2 3 CDSi 1.89 158 104 | | | 1.2 |
| | | | | CH.21_p2 gi\|4210430 | |
| 308157 | EOS08088 | AI510824 | Hs.75968 | thymosin; beta 4; X chromosome | 1.2 |
| 314452 | EOS14383 | AL042699 | Hs.209222 | ESTs | 1.2 |
| 308268 | EOS08199 | AI567509 | Hs.172928 | collagen; type I; alpha 1 | 1.2 |
| 321467 | EOS21398 | X13075 | | EST cluster (not in UniGene) | 1.2 |
| 320993 | EOS20924 | AL050145 | Hs.225986 | Homo sapiens mRNA; cDNA DKFZp586C2020 (from clone DKFZp586C2020) | 1.2 |
| 336778 | EOS36709 | CH22_4367FG_{—} | | CH22_FGENES.159-4 | 1.2 |
| | | | | | 159_4_{—} |
| 319827 | EOS19758 | T62778 | | EST cluster (not in UniGene) | 1.2 |
| 308249 | EOS08180 | AI560998 | | EST singleton (not in UniGene) with exon hit | 1.2 |
| 310094 | EOS10025 | AW450967 | Hs.235240 | ESTs | 1.2 |
| 336902 | EOS36833 | CH22_4655FG_{—} | | CH22_FGENES.331-2 | 1.2 |
| | | 331_2_{—} | | | |
| 339044 | EOS38975 | CH22_7944FG_LINK_DA59H18.GENSCAN.27-5 | | | 1.2 |
| | | | | CH22_DA59H18.GENSCAN.27-5 | |
| 336675 | EOS36606 | CH22_4153FG_43_3_{—} | | CH22_FGENES.43-3 | 1.2 |
| 303563 | EOS03494 | AA367699 | Hs.118787 | transforming growth factor; | 1.2 |
| | | | | beta-induced; 68 kD | |
| 330673 | EOS30604 | D57823 | Hs.92962 | Sec23 (S. cerevisiae) homolog A | 1.2 |
| 311814 | EOS11745 | AW377113 | Hs.119640 | ESTs; Moderately similar to zinc finger protein [H. sapiens] | 1.2 |
| 335481 | EOS35412 | CH22_2833FG_570_10_LINK_EM:AC005500.GENSCAN.460-4 | | | 1.2 |
| | | | | CH22_FGENES.570_10 | |
| 314775 | EOS14706 | AI149880 | Hs.188809 | ESTs | 1.2 |
| 324961 | EOS24892 | AA613792 | | EST cluster (not in UniGene) | 1.2 |
| 313458 | EOS13389 | AA007259 | Hs.255853 | ESTs | 1.2 |
| 307074 | EOS07005 | AI150989 | | EST singleton (not in UniGene) with exon hit | 1.2 |
| 337964 | EOS37895 | CH22_6410FG_LINK_EM:AC005500.GENSCAN.100-9 | | | 1.2 |
| | | | | CH22_EM:AC005500.GENSCAN.100-9 | |
| 326519 | EOS26450 | c19_hs gi\|5867439\|ref\|gn 4 + 166004 166243 ex 4 5 CDSi 4.49 240 2534 | | | 1.2 |
| | | | | CH.19_hs gi\|5867439 | |
| 337366 | EOS37297 | CH22_5551FG_{—} | | CH22_FGENES.736-1 | 1.2 |
| | | 736_1_{—} | | | |
| 322340 | EOS22271 | AF088076 | | EST cluster (not in UniGene) | 1.2 |
| 307954 | EOS07885 | AI419692 | | EST singleton (not in UniGene) with exon hit | 1.2 |
| 328615 | EOS28546 | c_7_hs gi\|5868239\|ref\|gn 2 + 35214 35347 ex 3 4 CDSi 11.49 134 3651 | | | 1.2 |
| | | | | CH.07_hs gi\|5868239 | |
| 317787 | EOS17718 | AW339612 | Hs.249364 | ESTs | 1.2 |
| 335288 | EOS35219 | CH22_2630FG_527_1_LINK_EM:AC005500.GENSCAN.421-1 | | | 1.2 |
| | | | | CH22_FGENES.527_1 | |
| 323175 | EOS23106 | AI827137 | Hs.184023 | ESTs | 1.2 |
| 330893 | EOS30824 | AA149620 | Hs.71999 | ESTs | 1.2 |
| 306810 | EOS06741 | AI057294 | | EST singleton (not in UniGene) with exon hit | 1.2 |
| 338239 | EOS38170 | CH22_6833FG_LINK_EM:AC005500.GENSCAN.264-5 | | | 1.2 |
| | | | | CH22_EM:AC005500.GENSCAN.264-5 | |
| 332347 | EOS32278 | W60326 | Hs.221716 | ESTs | 1.2 |
| 309782 | EOS09713 | AW275156 | Hs.156110 | Immunoglobulin kappa variable 1D-8 | 1.2 |
| 322518 | EOS22449 | AI133446 | | EST cluster (not in UniGene) | 1.2 |
| 301187 | EOS01118 | AA806542 | | EST cluster (not in UniGene) with exon hit | 1.2 |
| 312129 | EOS12060 | AW300867 | | EST cluster (not in UniGene) | 1.2 |
| 334714 | EOS34645 | CH22_2024FG_421_25_LINK_EM:AC005500.GENSCAN.282-25 | | | 1.2 |
| | | | | CH22_FGENES.421_25 | |
| 316586 | EOS16517 | AI205077 | Hs.144689 | ESTs | 1.2 |
| 320488 | EOS20419 | R31386 | | EST cluster (not in UniGene) | 1.2 |
| 327458 | EOS27389 | c_2_hs gi\|6004455\|ref\|gn 3 + 173257 173378 ex 5 7 CDSi 4.03 122 1184 | | | 1.2 |
| | | | | CH.02_hs gi\|6004455 | |

| | | | | |
|---|---|---|---|---|
| 336707 | EOS36638 | CH22_4212FG_64_3_{—} | CH22_FGENES.64-3 | 1.2 |

| 313561 | EOS13492 | AA040155 | | EST cluster (not in UniGene) | 1.2 |
|---|---|---|---|---|---|
| 330906 | EOS30837 | AA169498 | Hs.72804 | ESTs | 1.2 |
| 330987 | EOS30918 | H40988 | Hs.131965 | ESTs; Weakly similar to !!!! ALU SUBFAMILY J WARNING ENTRY !!!! [H. sapiens] | 1.2 |
| 325041 | EOS24972 | AI809182 | Hs.130907 | ESTs | 1.2 |
| 313225 | EOS13156 | AA502384 | Hs.151529 | ESTs | 1.2 |
| 305295 | EOS05226 | AA687131 | | EST singleton (not in UniGene) with exon hit | 1.2 |
| 306898 | EOS06827 | AI093383 | | EST singleton (not in UniGene) with exon hit | 1.2 |
| 326981 | EOS26912 | c21_hs gi\|6588016\|ref\|gn 3 + 105091 106038 ex 1 1 CDSo 122.69 948 567 | | | 1.2 |
| | | | | CH.21_hs gi\|6588016 | |
| 332225 | EOS32156 | N33213 | Hs.100425 | ESTs | 1.2 |
| 318802 | EOS18733 | R19443 | Hs.92414 | ESTs | 1.2 |
| 318413 | EOS18344 | AI138592 | Hs.144936 | ESTs | 1.2 |
| 312292 | EOS12223 | AW451893 | Hs.151124 | ESTs | 1.2 |
| 323753 | EOS23684 | AA327102 | | EST cluster (not in UniGene) | 1.2 |
| 313582 | EOS13513 | AW207684 | Hs.13583 | ESTs | 1.2 |
| 317836 | EOS17767 | AA983913 | Hs.128929 | ESTs | 1.2 |
| 332868 | EOS32799 | CH22_86FG_28_8_LINK_C20H12.GENSCAN.18-8 | | | 1.2 |
| | | | | CH22_FGENES.28_8 | |
| 336924 | EOS36855 | CH22_4699FG_{—} | | CH22_FGENES.347_9 | 1.2 |
| | | 347_9_{—} | | | |
| 327791 | EOS27722 | c_5_hs gi\|5867977\|ref\|gn 1 + 22491 22610 ex 6 7 CDSi 11.29 120 658 | | | 1.2 |
| | | | | CH.05_hs gi\|5867977 | |
| 330717 | EOS30648 | AA233926 | Hs.23635 | ESTs | 1.2 |
| 322944 | EOS22875 | AA112573 | | EST cluster (not in UniGene) | 1.2 |
| 312108 | EOS12039 | T82331 | Hs.127453 | ESTs | 1.2 |
| 332570 | EOS32501 | AA401376 | Hs.26176 | ESTs | 1.2 |
| 330880 | EOS30811 | AA132420 | Hs.53542 | KIAA0986 protein | 1.2 |
| 310341 | EOS10272 | AW302773 | | EST cluster (not in UniGene) | 1.2 |
| 334012 | EOS33943 | CH22_1290FG_313_3_LINK_EM:AC005500.GENSCAN.169-3 | | | 1.2 |
| | | | | CH22_FGENES.313_3 | |
| 318230 | EOS18161 | AA558125 | | EST cluster (not in UniGene) | 1.2 |
| 336071 | EOS36002 | CH22_3457FG_685_3_LINK_DJ32I10.GENSCAN.21-6 | | | 1.2 |
| | | | | CH22_FGENES.685_3 | |
| 338510 | EOS38441 | CH22_708FG_LINK_EM:AC005500.GENSCAN.391-22 | | | 1.2 |
| | | | | CH22_EM:AC005500.GENSCAN.391-22 | |
| 334487 | EOS34418 | CH22_1786FG_395_9_LINK_EM:AC005500.GENSCAN.258-10 | | | 1.2 |
| | | | | CH22_FGENES.395_9 | |
| 320661 | EOS20592 | AA864846 | | EST cluster (not in UniGene) | 1.2 |
| 335200 | EOS35131 | CH22_2538FG_508_9_LINK_EM:AC005500.GENSCAN.401-9 | | | 1.2 |
| | | | | CH22_FGENES.508_9 | |
| 333582 | EOS33513 | CH22_842FG_201_2_LINK_EM:AC005500.GENSCAN.72-3 | | | 1.2 |
| | | | | CH22.FGENES.201_2 | |
| 320789 | EOS20720 | R78712 | | EST cluster (not in UniGene) | 1.2 |
| 321185 | EOS21116 | H51659 | Hs.189854 | ESTs | 1.2 |
| 337740 | EOS37671 | CH22_6085FG_LINK_EM:AC000097.GENSCAN.100-6 | | | 1.2 |
| | | | | CH22_EM:AC000097.GENSCAN.100-6 | |
| 315064 | EOS14995 | AA775208 | Hs.136423 | ESTs | 1.2 |
| 334883 | EOS34814 | CH22_2197FG_451_6_LINK_EM:AC005500.GENSCAN.340-6 | | | 1.2 |
| | | | | CH22_FGENES.451_6 | |
| 331825 | EOS31756 | AA411144 | Hs.104768 | ESTs | 1.2 |
| 319141 | EOS19072 | F12377 | | EST cluster (not in UniGene) | 1.1 |
| 333682 | EOS33613 | CH22_944FG_247_10_LINK_EM:AC005500.GENSCAN.102-10 | | | 1.1 |
| | | | | CH22_FGENES.247_10 | |
| 336140 | EOS36071 | CH22_3530FG_705_2_LINK_DA59H18.GENSCAN.10-2 | | | 1.1 |
| | | | | CH22_FGENES.705_2 | |
| 320727 | EOS20658 | U96044 | | EST cluster (not in UniGene) | 1.1 |
| 323947 | EOS23878 | AA649842 | Hs.186667 | ESTs | 1.1 |
| 324746 | EOS24677 | AA603367 | Hs.222294 | ESTs | 1.1 |
| 306744 | EOS06675 | AI031882 | | EST singleton (not in UniGene) with exon hit | 1.1 |
| 326517 | EOS26448 | c19_hs gi\|5867439\|ref\|gn 1 + 44732 46356 ex 6 6 CDSl 148.22 1625 2512 | | | 1.1 |
| | | | | CH.19_hs gi\|5867439 | |
| 333597 | EOS33528 | CH22_858FG_211_5_LINK_EM:AC005500.GENSCAN.79-5 | | | 1.1 |
| | | | | CH22_FGENES.211_5 | |
| 330135 | EOS30066 | c21_p2 gi\|4456470\|emb\|gn 2 − 121583 121885 ex 2 2 CDSl 18.67 303 102 | | | 1.1 |
| | | | | CH.21_p2 gi\|4456470 | |
| 315118 | EOS15049 | AA564921 | Hs.143899 | ESTs | 1.1 |
| 302893 | EOS02824 | AL117539 | Hs.173515 | Homo sapiens mRNA; cDNA DKFZp586H021 (from clone DKFZp586H021) | 1.1 |
| 337169 | EOS37100 | CH22_5189FG_{—} | | CH22_FGENES.563-1 | 1.1 |
| | | 563_1_{—} | | | |
| 336121 | EOS36052 | CH22_3510FG_701_6_LINK_DA59H18.GENSCAN.8-6 | | | 1.1 |
| | | | | CH22_FGENES.701_6 | |
| 323332 | EOS23263 | AI829520 | Hs.227513 | ESTs | 1.1 |
| 320911 | EOS20842 | AI056872 | Hs.133386 | ESTs | 1.1 |
| 327990 | EOS27921 | c_6_hs gi\|5868218\|ref\|gn 2 − 36225 36503 ex 1 2 CDSl 16.35 279 1419 | | | 1.1 |
| | | | | CH.06_hs gi\|5868218 | |
| 320425 | EOS20356 | C14069 | Hs.201627 | ESTs; Moderately similar to !!!! ALU SUBFAMILY SQ WARNING | 1.1 |
| | | | | ENTRY !!!! [H. sapiens] | |
| 327075 | EOS27006 | c21_hs gi\|6531965\|ref\|gn 58 + 4041318 4041431 ex 4 4 CDSl 1.79 114 1285 | | | 1.1 |
| | | | | CH.21_hs gi\|6531965 | |
| 314384 | EOS14315 | AA535840 | Hs.162203 | ESTs; Weakly similar to alternatively spliced product using exon 13A [H. sapiens] | 1.1 |
| 338716 | EOS38647 | CH22_7502FG_LINK_EM:AC005500.GENSCAN.488-9 | | | 1.1 |
| | | | | CH22_EM:AC005500.GENSCAN.488-9 | |
| 330886 | EOS30817 | AA135606 | Hs.189384 | ESTs; Weakly similar to !!!! ALU SUBFAMILY J WARNING ENTRY !!!! [H. sapiens] | 1.1 |
| 327331 | EOS27262 | c_1_hs gi\|5867516\|ref\|gn 4 − 55606 55737 ex 2 6 CDSi 7.01 132 2349 | | | 1.1 |
| | | | | CH.01_hs gi\|5867516 | |
| 326714 | EOS26645 | c20_hs gi\|5867595\|ref\|gn 2 + 124490 124568 ex 5 6 CDSi 0.11 79 1020 | | | 1.1 |
| | | | | CH.20_hs gi\|5867595 | |
| 316734 | EOS16665 | AW080237 | Hs.252884 | ESTs | 1.1 |
| 311660 | EOS11591 | AI978583 | Hs.232161 | ESTs | 1.1 |
| 312757 | EOS12688 | AI285970 | Hs.183817 | ESTs | 1.1 |
| 331686 | EOS31617 | W88502 | Hs.182258 | ESTs | 1.1 |
| 337840 | EOS37771 | CH22_6223FG_LINK_EM:AC005500.GENSCAN.26-9 | | | 1.1 |
| | | | | CH22.EM:AC005500.GENSCAN.26-9 | |
| 332093 | EOS32024 | AA808794 | Hs.112592 | ESTs | 1.1 |
| 319595 | EOS19526 | H81361 | Hs.194485 | ESTs | 1.1 |
| 315990 | EOS15921 | AI800041 | Hs.190555 | ESTs | 1.1 |
| 322438 | EOS22369 | W44531 | Hs.167851 | ESTs | 1.1 |
| 332965 | EOS32896 | CH22_189FG_50_3_LINK_EM:AC000097.GENSCAN.3-5 | | | 1.1 |
| | | | | CH22_FGENES.50_3 | |
| 337182 | EOS37113 | CH22_5204FG_{—} | | CH22_FGENES.570-2 | 1.1 |
| | | 570_2_{—} | | | |
| 334948 | EOS34879 | CH22_2269FG_465_15_LINK_EM:AC005500.GENSCAN.359-13 | | | 1.1 |
| | | | | CH22_FGENES.465_15 | |
| 325864 | EOS25795 | c16_hs gi\|5867069\|ref\|gn 2 − 110834 110904 ex 3 3 CDSf 9.76 71 457 | | | 1.1 |
| | | | | CH.16_hs gi\|5867069 | |
| 337760 | EOS37691 | CH22_6110FG_LINK_EM:AC000097.GENSCAN.116-8 | | | 1.1 |
| | | | | CH22_EM:AC000097.GENSCAN.116-8 | |
| 315422 | EOS15353 | AW135357 | Hs.192374 | ESTs | 1.1 |
| 338889 | EOS38820 | CH22_7746FG_LINK_DJ32I10.GENSCAN.7-1 | | | 1.1 |
| | | | | CH22_DJ32I10.GENSCAN.7-1 | |
| 332961 | EOS32892 | CH22_185FG_48_18_LINK_EM:AC000097.GENSCAN.2-14 | | | 1.1 |
| | | | | CH22_FGENES.48_18 | |
| 314703 | EOS14634 | AI791249 | | EST cluster (not in UniGene) | 1.1 |
| 317791 | EOS17722 | AI801500 | Hs.128457 | ESTs | 1.1 |
| 333680 | EOS33611 | CH22_942FG_247_7_LINK_EM:AC005500.GENSCAN.102-7 | | | 1.1 |
| | | | | CH22_FGENES.247_7 | |
| 322419 | EOS22350 | AA248987 | Hs.14084 | ESTs; Highly similar to zinc RING finger protein SAG [M. musculus] | 1.1 |
| 338124 | EOS38055 | CH22_6561FG_LINK_EM:AC005500.GENSCAN.196-2 | | | 1.1 |
| | | | | CH22_EM:AC005500.GENSCAN.196-2 | |
| 308884 | EOS08815 | AI833131 | Hs.179100 | ESTs | 1.1 |
| 333349 | EOS33280 | CH22_595FG_140_3_LINK_EM:AC005500.GENSCAN.20-3 | | | 1.1 |
| | | | | CH22_FGENES.140_3 | |
| 313150 | EOS13081 | AA824410 | Hs.165003 | ESTs | 1.1 |
| 339208 | EOS39139 | CH22_8146FG_LINK_FF113D11.GENSCAN.6-3 | | | 1.1 |
| | | | | CH22_FF113D11.GENSCAN.6-3 | |
| 335653 | EOS35584 | CH22_3013FG_590_4_LINK_EM:AC005500.GENSCAN.484-4 | | | 1.1 |
| | | | | CH22_FGENES.590_4 | |
| 319524 | EOS19455 | AA682865 | Hs.194441 | ESTs | 1.1 |
| 301576 | EOS01507 | AI682905 | Hs.146875 | ESTs; Weakly similar to !!!! ALU SUBFAMILY J WARNING ENTRY !!!! [H. sapiens] | 1.1 |
| 317598 | EOS17529 | AW206035 | Hs.192123 | ESTs | 1.1 |
| 333473 | EOS33404 | CH22_724FG_162_3_LINK_EM:AC005500.GENSCAN.42-10 | | | 1.1 |
| | | | | CH22_FGENES.162_3 | |
| 333949 | EOS33880 | CH22_1225FG_303_5_LINK_EM:AC005500.GENSCAN.162-9 | | | 1.1 |
| | | | | CH22_FGENES.303_5 | |
| 339256 | EOS39187 | CH22_8207FG_LINK_BA354I12.GENSCAN.7-11 | | | 1.1 |
| | | | | CH22_BA354I12.GENSCAN.7-11 | |
| 332884 | EOS32815 | CH22_104FG_33_5_LINK_C20H12.GENSCAN.22-7 | | | 1.1 |
| | | | | CH22_FGENES.33_5 | |
| 314660 | EOS14591 | AA436007 | Hs.188780 | ESTs | 1.1 |
| 333220 | EOS33151 | CH22_457FG_104_12_LINK_EM:AC000097.GENSCAN.108-11 | | | 1.1 |
| | | | | CH22_FGENES.104_12 | |
| 308106 | EOS08037 | AI476803 | | EST singleton (not in UniGene) with exon hit | 1.1 |
| 320709 | EOS20640 | AA456660 | Hs.154165 | ESTs | 1.1 |
| 307612 | EOS07543 | AI290787 | | EST singleton (not in UniGene) with exon hit | 1.1 |
| 330286 | EOS30217 | c_5_p2 gi\|6671913\|gb\|A gn 2 − 31050 31171 ex 2 7 CDSi 8.84 122 791 | | | 1.1 |
| | | | | CH.05_p2 gi\|6671913 | |
| 304495 | EOS04426 | AA446448 | | EST singleton (not in UniGene) with exon hit | 1.1 |
| 310583 | EOS10514 | AW205632 | Hs.211198 | ESTs | 1.1 |
| 332896 | EOS32827 | CH22_117FG_35_10_LINK_C20H12.GENSCAN.24-9 | | | 1.1 |
| | | | | CH22_FGENES.35_10 | |
| 337602 | EOS37533 | CH22_5895FG_LINK_C20H12.GENSCAN.15-1 | | | 1.1 |
| | | | | CH22_C20H12.GENSCAN.15-1 | |
| 307626 | EOS07557 | AI300035 | | EST singleton (not in UniGene) with exon hit | 1.1 |
| 334696 | EOS34627 | CH22_2006FG_421_5_LINK_EM:AC005500.GENSCAN.282-5 | | | 1.1 |
| | | | | CH22_FGENES.421_5 | |
| 318652 | EOS18583 | T53259 | | EST cluster (not in UniGene) | 1.1 |
| 337844 | EOS37775 | CH22_6229FG_LINK_EM:AC005500.GENSCAN.30-9 | | | 1.1 |
| | | | | CH22_EM:AC005500.GENSCAN.30-9 | |
| 334823 | EOS34754 | CH22_2137FG_437_5_LINK_EM:AC005500.GENSCAN.301-7 | | | 1.1 |
| | | | | CH22_FGENES.437_5 | |
| 333928 | EOS33859 | CH22_1201FG_299_2_LINK_EM:AC005500.GENSCAN.158-5 | | | 1.1 |
| | | | | CH22_FGENES.299_2 | |
| 337503 | EOS37434 | CH22_5738FG_{—} | | CH22_FGENES.803-1 | 1.1 |
| | | 803_1_{—} | | | |
| 323044 | EOS22975 | AA148725 | Hs.154190 | ESTs | 1.1 |
| 329164 | EOS29095 | c_x_hs gi\|5868691\|ref\|gn 1 + 62305 62517 ex 2 2 CDSl 17.51 213 1868 | | | 1.1 |
| | | | | CH.X_hs gi\|5868691 | |
| 335468 | EOS35399 | CH22_2819FG_567_4_LINK_EM:AC005500.GENSCAN.454-12 | | | 1.1 |
| | | | | CH22_FGENES.567_4 | |
| 338962 | EOS38893 | CH22_7838FG_LINK_DJ32I10.GENSCAN.23-39 | | | 1.1 |
| | | | | CH22_DJ32I10.GENSCAN.23-39 | |
| 323570 | EOS23501 | AL038623 | Hs.208752 | ESTs; Weakly similar to !!!! ALU SUBFAMILY SX WARNING ENTRY !!!! [H. sapiens] | 1.1 |
| 333568 | EOS33499 | CH22_826FG_185_1_LINK_EM:AC005500.GENSCAN.64-1 | | | 1.1 |
| | | | | CH22_FGENES.185_1 | |
| 331865 | EOS31796 | AA425756 | Hs.98445 | ESTs | 1.1 |
| 336246 | EOS36177 | CH22_3644FG_746_5_LINK_DA59H18.GENSCAN.48-4 | | | 1.1 |
| | | | | CH22_FGENES.746_5 | |
| 337238 | EOS37169 | CH22_5343FG_{—} | | CH22_FGENES.641-3 | 1.1 |
| | | 641_3_{—} | | | |
| 305089 | EOS05020 | AA642622 | | EST singleton (not in UniGene) with exon hit | 1.1 |
| 300097 | EOS00028 | AI916973 | Hs.213603 | ESTs | 1.1 |
| 313134 | EOS13065 | N63406 | Hs.258697 | ESTs | 1.1 |
| 337452 | EOS37383 | CH22_5665FG_{—} | | CH22_FGENES.775-1 | 1.1 |
| | | 775_1_{—} | | | |
| 325433 | EOS25364 | c12_hs gi\|5866936\|ref\|gn 4 − 480706 480826 ex 3 4 CDSi 1.99 121 818 | | | 1.1 |
| | | | | CH.12_hs gi\|5866936 | |
| 335999 | EOS35930 | CH22_3380FG_657_1_LINK_DJ246D7.GENSCAN.11-1 | | | 1.1 |
| | | | | CH22_FGENES.657_1 | |
| 333580 | EOS33511 | CH22_840FG_199_2_LINK_EM:AC005500.GENSCAN.71-2 | | | 1.1 |
| | | | | CH22_FGENES.199_2 | |
| 336836 | EOS36767 | CH22_4512FG_{—} | | CH22_FGENES.247-11 | 1.1 |
| | | 247_11_{—} | | | |
| 334677 | EOS34608 | CH22_1986FG_418_30_LINK_EM:AC005500.GENSCAN.279-31 | | | 1.1 |
| | | | | CH22_FGENES.418_30 | |
| 329062 | EOS28993 | c_x_hs gi\|5868590\|ref\|gn 3 − 58977 59094 ex 4 11 CDSi − 6.19 118 627 | | | 1.1 |
| | | | | CH.X_hs gi\|5868590 | |
| 333671 | EOS33602 | CH22_932FG_245_5_LINK_EM:AC005500.GENSCAN.100-12 | | | 1.1 |
| | | | | CH22_FGENES.245_5 | |
| 304941 | EOS04872 | AA612612 | | EST singleton (not in UniGene) with exon hit | 1.1 |
| 315772 | EOS15703 | AW515373 | Hs.158893 | ESTs | 1.1 |
| 301281 | EOS01212 | AA843986 | Hs.190586 | ESTs | 1.1 |
| 333520 | EOS33451 | CH22_777FG_174_3_LINK_EM:AC005500.GENSCAN.53-6 | | | 1.1 |
| | | | | CH22_FGENES.174_3 | |
| 315203 | EOS15134 | AI559820 | Hs.199438 | ESTs | 1.1 |
| 315927 | EOS15858 | AW025517 | Hs.133250 | ESTs | 1.1 |
| 317161 | EOS17092 | AA972165 | Hs.150308 | ESTs | 1.1 |
| 337692 | EOS37623 | CH22_6028FG_LINK_EM:AC000097.GENSCAN.78-12 | | | 1.1 |
| | | | | CH22_EM:AC000097.GENSCAN.78-12 | |
| 331472 | EOS31403 | N24830 | | yx70a02.s1 Soares melanocyte 2NbHM Homo sapiens cDNA clone | 1.1 |
| | | | | IMAGE:267050 3′ similar to gb\|M87912\|HUMALNE562 | |
| | | | | Human carcinoma cell-derived Alu RNA transcript, (rRNA); contains Alu | |
| | | | | repetitive element;, mRNA sequence. | |
| 336439 | EOS36370 | CH22_3859FG_827_4_LINK_DJ579N16.GENSCAN.1-3 | | | 1.1 |
| | | | | CH22_FGENES.827_4 | |
| 326882 | EOS26813 | c20_hs gi\|6682509\|ref\|gn 2 − 167988 168179 ex 4 4 CDSf 18.69 192 2238 | | | 1.1 |
| | | | | CH.20_hs gi\|6682509 | |
| 336977 | EOS36908 | CH22_4793FG_{—} | | CH22_FGENES.380-9 | 1.1 |
| | | 380_9_{—} | | | |
| 333983 | EOS33914 | CH22_1260FG_310_LINK_EM:AC005500.GENSCAN.167-5 | | | 1.1 |
| | | | | CH22_FGENES.310_7 | |
| 328878 | EOS28809 | c_7_hs gi\|6552423\|ref\|gn 1 + 105580 105774 ex 6 7 CDSi 2.91 195 6195 | | | 1.1 |
| | | | | CH.07_hs gi\|6552423 | |
| 330415 | EOS30346 | D83777 | Hs.75137 | KIAA0193 gene product | 1.1 |
| 324824 | EOS24755 | AI826999 | Hs.224624 | ESTs | 1.1 |
| 325815 | EOS25746 | c14_hs gi\|6682483\|ref\|gn 1 − 129273 130754 ex 1 1 CDSo 11.82 1482 2225 | | | 1.1 |
| | | | | CH.14_hs gi\|6682483 | |
| 300463 | EOS00394 | N52510 | Hs.186470 | ESTs | 1.1 |
| 335708 | EOS35639 | CH22_3069FG_599_8_LINK_EM:AC005500.GENSCAN.490-11 | | | 1.1 |
| | | | | CH22_FGENES.599_8 | |
| 324575 | EOS24506 | AW502257 | | EST cluster (not in UniGene) | 1.1 |
| 337951 | EOS37882 | CH22_6391FG_LINK_EM:AC005500.GENSCAN.94-1 | | | 1.1 |
| | | | | CH22_EM:AC005500.GENSCAN.94-1 | |
| 335935 | EOS35866 | CH22_3313FG_646_6_LINK_DJ246D7.GENSCAN.1-5 | | | 1.1 |
| | | | | CH22_FGENES.646_6 | |
| 334914 | EOS34845 | CH22_2233FG_457_3_LINK_EM:AC005500.GENSCAN.346-2 | | | 1.1 |
| | | | | CH22_FGENES.457_3 | |
| 309527 | EOS09458 | AW150648 | Hs.75621 | protease inhibitor 1 (anti-elastase); alpha-1-antitrypsin | 1.1 |
| 318901 | EOS18832 | AW368520 | Hs.24639 | ESTs | 1.1 |
| 320484 | EOS20415 | AA094436 | Hs.155712 | follistatin-like 1 | 1.1 |
| 333665 | EOS33596 | CH22_926FG_244_1_LINK_EM:AC005500.GENSCAN.99-1 | | | 1.1 |
| | | | | CH22_FGENES.244_1 | |
| 335860 | EOS35791 | CH22_3235FG_629_5_LINK_EM:AC005500.GENSCAN.519-4 | | | 1.1 |
| | | | | CH22_FGENES.629-5 | |
| 313339 | EOS13270 | AI682536 | Hs.163495 | ESTs | 1.1 |
| 300149 | EOS00080 | AW448916 | Hs.149018 | ESTs | 1.1 |
| 318112 | EOS18043 | AI028162 | Hs.132307 | ESTs | 1.1 |
| 337807 | EOS37738 | CH22_6178FG_LINK_EM:AC005500.GENSCAN.9-4 | | | 1.1 |
| | | | | CH22_EM:AC005500.GENSCAN.9-4 | |
| 336917 | EOS36848 | CH22_4688FG_{—} | | CH22_FGENES.346-4 | 1.1 |
| | | 346_4_{—} | | | |
| 337489 | EOS37420 | CH22_5722FG_{—} | | CH22_FGENES.799-2 | 1.1 |
| | | 799_2_{—} | | | |
| 320112 | EOS20043 | T92107 | Hs.188489 | ESTs | 1.1 |
| 332975 | EOS32906 | CH22_199FG_51_10_LINK_EM:AC000097.GENSCAN.4-12 | | | 1.1 |
| | | | | CH22_FGENES.51_10 | |
| 327805 | EOS27736 | c_5_hs gi\|5867968\|ref gn 2 + 19952 20019 ex 1 2 CDSf 9.47 68 988 | | | 1.1 |
| | | | | CH.05_hs gi\|5867968 | |
| 339215 | EOS39146 | CH22_8153FG_LINK_FF113D11.GENSCAN.6-10 | | | 1.1 |
| | | | | CH22_FF113D11.GENSCAN.6-10 | |
| 311965 | EOS11896 | T69279 | | EST clusler (not in UniGene) | 1.1 |
| 314043 | EOS13974 | AA827082 | | EST cluster (not in UniGene) | 1.1 |
| 333447 | EOS33378 | CH22_697FG_154_5_LINK_EM:AC005500.GENSCAN.35-6 | | | 1.1 |
| | | | | CH22_FGENES.154_5 | |
| 333242 | EOS33173 | CH22_481FG_111_6_LINK_EM:AC000097.GENSCAN.120-5 | | | 1.1 |
| | | | | CH22_FGENES.111_6 | |
| 338596 | EOS38527 | CH22_7343FG_LINK_EM:AC005500.GENSCAN.437-2 | | | 1.1 |
| | | | | CH22_EM:AC005500.GENSCAN.437-2 | |
| 329989 | EOS29920 | c16_p2 gi\|4567166\|gb\|Agn 2 + 72861 73052 ex 1 3 CDSf 18.02 192 590 | | | 1.1 |
| | | | | CH.16_p2 gi\|4567166 | |
| 315675 | EOS15606 | AA652272 | Hs.197320 | ESTs | 1.1 |
| 336722 | EOS36653 | CH22_4245FG_{—} | | CH22_FGENES.84-2 | |
| | | 84_2_{—} | | | |
| 334220 | EOS34151 | CH22_1503FG_359_4_LINK_EM:AC005500.GENSCAN.217-7 | | | 1.1 |
| | | | | CH22_FGENES.359_4 | |
| 336703 | EOS36634 | CH22_4201FG_{—} | | CH22_FGENES.56-3 | 1.1 |
| | | 56_3_{—} | | | |
| 336397 | EOS36328 | CH22_3812FG_823_12_LINK_BA232E17.GENSCAN.6-11 | | | 1.1 |
| | | | | CH22_FGENES.823_12 | |
| 316105 | EOS16036 | AW295687 | Hs.254420 | ESTs | 1.1 |
| 334661 | EOS34592 | CH22_1969FG_418_9_LINK_EM:AC005500.GENSCAN.279-13 | | | 1.1 |
| | | | | CH22_FGENES.418_9 | |
| 307783 | EOS07714 | AI347274 | | EST singleton (not in UniGene) with exon hit | 1.1 |
| 333997 | EOS33928 | CH22_1275FG_310_22_LINK_EM:AC005500.GENSCAN.167-21 | | | 1.1 |
| | | | | CH22_FGENES.310_22 | |
| 331903 | EOS31834 | AA436673 | Hs.29417 | Homo sapiens mRNA; cDNA DKFZp586B0323 (from clone DKFZp586B0323) | 1.1 |
| 328249 | EOS28180 | c_6_hs gi\|6381891\|ref\|gn2 − 96352 96527 ex 2 3 CDSi 6.19 176 4550 | | | 1.1 |
| | | | | CH_06_hs gi\|6381891 | |
| 338251 | EOS38182 | CH22_6849FG_LINK_EM:AC005500.GENSCAN.270-1 | | | 1.1 |
| | | | | CH22_EM:AC005500.GENSCAN.270-1 | |
| 323561 | EOS23492 | AA825426 | Hs.238832 | ESTs; Weakly similar to !!!! ALU SUBFAMILY J WARNING ENTRY !!!! [H. sapiens] | 1.1 |
| 301464 | EOS01395 | AA991519 | Hs.253324 | ESTs | 1.1 |
| 335916 | EOS35847 | CH22_3293FG_636_12_LINK_EM:AC005500.GENSCAN.526-12 | | | 1.1 |
| | | | | CH22_FGENES.636_12 | |
| 321828 | EOS21759 | X56197 | | EST cluster (not in UniGene) | 1.1 |
| 327413 | EOS27344 | c_2_hs gi\|5867750\|ref\|gn 3 + 101410 101508 ex 4 5 CDSl 4.34 99 587 | | | 1.1 |
| | | | | CH.02_hs gi\|5867750 | |
| 334474 | EOS34405 | CH22_1773FG_394_5_LINK_EM:AC005500.GENSCAN.257-5 | | | 1.1 |
| | | | | CH22_FGENES.394_5 | |
| 336739 | EOS36670 | CH22_4291FG_{—} | | CH22_FGENES.117-3 | 1.1 |
| | | 117_3_{—} | | | |
| 316517 | EOS16448 | AI784315 | Hs.123163 | ESTs | 1.1 |
| 325519 | EOS25450 | c12_hs gi\|6017036\|ref\|gn 5 − 186804 186915 ex 1 3 CDSl 8.36 112 2508 | | | 1.1 |
| | | | | CH.12_hs gi\|6017036 | |
| 333875 | EOS33806 | CH22_1145FG_291_11_LINK_EM:AC005500.GENSCAN.149-6 | | | 1.1 |
| | | | | CH22_FGENES.291_11 | |
| 338221 | EOS38152 | CH22_6797FG_LINK_EM:AC005500.GENSCAN.246-10 | | | 1.1 |
| | | | | CH22_EM:AC005500.GENSCAN.246-10 | |
| 336878 | EOS36809 | CH22_4617FG_{—} | | CH22_FGENES.318-5 | 1.1 |
| | | 318_5_{—} | | | |
| 337919 | EOS37850 | CH22_6338FG_LINK_EM:AC005500.GENSCAN.66-5 | | | 1.1 |
| | | | | CH22_EM:AC005500.GENSCAN.66-5 | |
| 309828 | EOS09759 | AW293999 | | EST singleton (not in UniGene) with exon hit | 1.1 |
| 305259 | EOS05190 | AA679225 | | EST singleton (not in UniGene) with exon hit | 1.1 |
| 333922 | EOS33853 | CH22_1195FG_296_13_LINK_EM:AC005500.GENSCAN.155-16 | | | 1.1 |
| | | | | CH22_FGENES.296_13 | |
| 322092 | EOS22023 | AF085833 | | EST cluster (not in UniGene) | 1.1 |
| 313356 | EOS13287 | AI266254 | Hs.132929 | ESTs | 1.1 |
| 318847 | EOS18778 | Z42908 | Hs.12308 | ESTs | 1.1 |
| 337175 | EOS37106 | CH22_5195FG_{—} | | CH22_FGENES.567-1 | 1.1 |
| | | 567_1_{—} | | | |
| 336979 | EOS36910 | CH22_4802FG_{—} | | CH22_FGENES.385-4 | 1.1 |
| | | 385_A_{—} | | | |
| 312169 | EOS12100 | AI064824 | Hs.193385 | ESTs | 1.1 |
| 336198 | EOS36129 | CH22_3595FG_719_2_LINK_DA59H18.GENSCAN.21-2 | | | 1.1 |
| | | | | CH22_FGENES.719_2 | |
| 321948 | EOS21879 | AA309612 | Hs.118797 | ubiquitin-conjugating enzyme E2D 3 (homologous to yeast UBC4/5) | 1.1 |
| 324692 | EOS24623 | AA557952 | | EST cluster (not in UniGene) | 1.1 |
| 330395 | EOS30326 | D10923 | Hs.137555 | putative chemokine receptor; GTP-binding protein | 1.1 |
| 333119 | EOS33050 | CH22_347FG_80_4_LINK_EM:AC000097.GENSCAN.65-4 | | | 1.1 |
| | | | | CH22_FGENES.80_4 | |
| 316012 | EOS15943 | AA764950 | Hs.119898 | ESTs | 1.1 |
| 300142 | EOS00073 | AI743419 | Hs.205707 | ESTs | 1.1 |
| 317215 | EOS17146 | AW014242 | Hs.159998 | ESTs | 1.1 |
| 329526 | EOS29457 | c10_p2 gi\|3983506\|gb\|U gn 2 + 12251 12325 ex 3 3 CDSl 7.37 75 178 | | | 1.1 |
| | | | | CH.10_p2 gi\|3983506 | |
| 317409 | EOS17340 | AA764968 | Hs.4864 | KIAA0892 protein | 1.1 |
| 339230 | EOS39161 | CH22_8171FG_LINK_BA354I12.GENSCAN.1-6 | | | 1.1 |
| | | | | CH22_BA354I12.GENSCAN.1-6 | |
| 311598 | EOS11529 | AW023595 | Hs.232048 | ESTs | 1.1 |
| 339164 | EOS39095 | CH22_8091FG_LINK_DA59H18.GENSCAN.69-4 | | | 1.1 |
| | | | | CH22_DA59H18.GENSCAN.69-4 | |
| 326725 | EOS26656 | c20_hs gi\|6552456\|ref\|gn 2 − 223005 223125 ex 5 6 CDSi 6.10 121 1038 | | | 1.1 |
| | | | | CH.20_hs gi\|6552456 | |
| 330952 | EOS30883 | H02855 | Hs.29567 | ESTs | 1.1 |
| 334621 | EOS34552 | CH22_1928FG_412_4_LINK_EM:AC005500.GENSCAN.275-4 | | | 1.1 |
| | | | | CH22_FGENES.412_4 | |
| 301685 | EOS01616 | W67730 | | EST cluster (not in UniGene) with exon hit | 1.1 |
| 308781 | EOS08712 | AI811707 | | EST singleton (not in UniGene) with exon hit | 1.1 |
| 323413 | EOS23344 | AA248828 | Hs.225676 | ESTs | 1.1 |
| 306723 | EOS06654 | AI026151 | | EST singleton (not in UniGene) with exon hit | 1.1 |
| 331258 | EOS31189 | Z41777 | Hs.27413 | ESTs | 1.1 |
| 313028 | EOS12959 | AI355433 | Hs.190858 | ESTs | 1.1 |
| 333002 | EOS32933 | CH22_226FG_59_3_LINK_EM:AC000097.GENSCAN.21-3 | | | 1.1 |
| | | | | CH22_FGENES.59_3 | |
| 303011 | EOS02942 | AF090405 | | EST cluster (not in UniGene) with exon hit | 1.1 |
| 317687 | EOS17618 | AA972990 | Hs.127904 | ESTs | 1.1 |
| 328779 | EOS28710 | c_7_hs gi\|5868309\|ref\|gn 4 + 41570 41639 ex 1 5 CDSf 2.65 70 5365 | | | 1.1 |
| | | | | CH.07_hs gi\|5868309 | |
| 338707 | EOS38638 | CH22_7487FG_LINK_EM:AC005500.GENSCAN.482-2 | | | 1.1 |
| | | | | CH22_EM:AC005500.GENSCAN.482-2 | |
| 337974 | EOS37905 | CH22_6427FG_LINK_EM:AC005500.GENSCAN.106-3 | | | 1.1 |
| | | | | CH22_EM:AC005500.GENSCAN.106-3 | |
| 332854 | EOS32785 | CH22_71FG_22_1_LINK_C20H12.GENSCAN.15-2 | | | 1.1 |
| | | | | CH22_FGENES.22_1 | |
| 311225 | EOS11156 | AW451982 | Hs.248613 | ESTs | 1.1 |
| 337094 | EOS37025 | CH22_5018FG_{—} | | CH22_FGENES.465-19 | 1.1 |
| | | 465_19_{—} | | | |
| 319357 | EOS19288 | F13425 | Hs.26229 | ESTs | 1.1 |
| 332958 | EOS32889 | CH22_182FG_48_15_LINK_EM:AC000097.GENSCAN.2-11 | | | 1.1 |
| | | | | CH22_FGENES.48_15 | |
| 309634 | EOS09565 | AW193825 | | EST singleton (not in UniGene) with exon hit | 1.1 |
| 321171 | EOS21102 | AI769410 | Hs.221461 | ESTs | 1.1 |
| 316440 | EOS16371 | AI954795 | Hs.156135 | ESTs | 1.1 |
| 311665 | EOS11596 | AW294254 | Hs.223742 | ESTs | 1.1 |
| 327548 | EOS27479 | c_3_hs gi\|5867797\|ref\|gn 2 − 81067 81130 ex 3 7 CDSi 6.42 64 12 | | | 1.1 |
| | | | | CH.03_hs gi\|5867797 | |
| 314940 | EOS14871 | AW452768 | Hs.162045 | ESTs | 1.1 |
| 326401 | EOS26332 | c19_hs gi\|5867355\|ref\|gn 1 + 35165 35332 ex 9 11 CDSi 0.41 168 768 | | | 1.1 |
| | | | | CH.19_hs gi\|5867355 | |
| 336347 | EOS36278 | CH22_3759FG_815_3_LINK_BA232E17.GENSCAN.1-24 | | | 1.1 |
| | | | | CH22_FGENES.815_3 | |
| 322297 | EOS22228 | W76548 | Hs.136026 | ESTs; Moderately similar to !!!! ALU SUBFAMILY SC WARNING ENTRY !!!! | 1.1 |
| | | | | [H. sapiens] | |
| 309977 | EOS09908 | AW451663 | | EST singleton (not in UniGene) with exon hit | 1.1 |
| 333466 | EOS33397 | CH22_717FG_161_2_LINK_EM:AC005500.GENSCAN.42-2 | | | 1.1 |
| | | | | CH22_FGENES.161_2 | |
| 329170 | EOS29101 | c_x_hs gi\|5868693\|ref\|gn 2 + 67924 68019 ex 6 8 CDSi 3.30 96 1882 | | | 1.1 |
| | | | | CH.X_hs gi\|5868693 | |
| 329479 | EOS29410 | c10_p2 gi\|3983526\|gb\|A gn 3 − 7425 7561 ex 1 3 CDSl 4.33 137 22 | | | 1.1 |
| | | | | CH.10_p2 gi\|3983526 | |
| 326668 | EOS26599 | c20_hs gi\|6552455\|ref\|gn 1 + 146726 146838 ex 11 11 CDSl 1.84 113 767 | | | 1.1 |
| | | | | CH.20_hs gi\|6552455 | |
| 319364 | EOS19295 | H06538 | Hs.12270 | ESTs | 1.1 |
| 302988 | EOS02919 | W23986 | Hs.34578 | alpha2;3-sialyltransferase | 1.1 |
| 327687 | EOS27618 | c_4_hs gi\|5867847\|ref\|gn 1 − 169293 169362 ex 2 3 CDSi -0.28 70 782 | | | 1.1 |
| | | | | CH.04_hs gi\|5867847 | |
| 339413 | EOS39344 | CH22_8405FG_LINK_DJ579N16.GENSCAN.5-8 | | | 1.1 |
| | | | | CH22_DJ579N16.GENSCAN.5-8 | |
| 306156 | EOS06087 | AA918274 | Hs.76067 | heat shock 27 kD protein 1 | 1.1 |
| 320858 | EOS20789 | D59968 | | EST cluster (not in UniGene) | 1.1 |
| 325447 | EOS25378 | c12_hs gi\|5866941\|ref\|gn 3 − 372480 372621 ex 2 3 CDSi 9.16 142 1026 | | | 1.1 |
| | | | | CH.12_hs gi\|5866941 | |
| 322696 | EOS22627 | AI064724 | Hs.228468 | ESTs | 1.1 |
| 329959 | EOS29890 | c16_p2 gi\|5103803\|gb\|A gn 3 + 188050 188193 ex 8 8 CDSl 2.01 144 361 | | | 1.1 |
| | | | | CH.16_p2 gi\|5103803 | |
| 312628 | EOS12559 | AA632817 | Hs.190316 | ESTs | 1.1 |
| 339305 | EOS39236 | CH22_8262FG_LINK_BA354I12.GENSCAN.21-3 | | | 1.1 |
| | | | | CH22_BA354I12.GENSCAN.21-3 | |
| 311829 | EOS11760 | AI078483 | Hs.134549 | ESTs | 1.1 |
| 303270 | EOS03201 | AL120518 | Hs.105352 | ESTs | 1.1 |
| 321226 | EOS21157 | AA311443 | Hs.251416 | Homo sapiens mRNA; cDNA DKFZp586E2317 (from clone DKFZp586E2317) | 1.1 |
| 335827 | EOS35758 | CH22_3200FG_620_1_LINK_EM:AC005500.GENSCAN.512-1 | | | 1.1 |
| | | | | CH22_FGENES.620_1 | |
| 336677 | EOS36608 | CH22_4155FG_{—} | | CH22_FGENES.43-5 | 1.1 |
| | | 43_5_{—} | | | |
| 330081 | EOS30012 | c19_p2 gi\|6015314\|gb\|A gn 1 − 5768 5835 ex 4 9 CDSi 2.88 68 162 | | | 1.1 |
| | | | | CH.19_p2 gi\|6015314 | |
| 339313 | EOS39244 | CH22_8272FG_LINK_BA354I12.GENSCAN.22-11 | | | 1.1 |
| | | | | CH22_BA354I12.GENSCAN.22-11 | |
| 319936 | EOS19867 | W22152 | | EST cluster (not in UniGene) | 1.1 |
| 332858 | EOS32789 | CH22_76FG_24_1_LINK_C20H12.GENSCAN.16-6 | | | 1.1 |
| | | | | CH22_FGENES.24_1 | |
| 315630 | EOS15561 | AA648355 | Hs.185155 | ESTs; Weakly similar to echinoderm microtubule-associated protein-like | 1.1 |
| | | | | EMAP2 [H. sapiens] | |
| 332995 | EOS32926 | CH22_219FG_58_2_LINK_EM:AC000097.GENSCAN.19-2 | | | 1.1 |
| | | | | CH22_FGENES.58_2 | |
| 333441 | EOS33372 | CH22_691FG_151_5_LINK_EM:AC005500.GENSCAN.32-5 | | | 1.1 |
| | | | | CH22_FGENES.151_5 | |
| 333496 | EOS33427 | CH22_748FG_168_6_LINK_EM:AC005500.GENSCAN.47-5 | | | 1.1 |
| | | | | CH22_FGENES.168_6 | |
| 339188 | EOS39119 | CH22_8123FG_LINK_DA59H18.GENSCAN.72-16 | | | 1.1 |
| | | | | CH22_DA59H18.GENSCAN.72-16 | |
| 336981 | EOS36912 | CH22_4818FG_{—} | | CH22_FGENES.397-7 | 1.1 |
| | | 397_7_{—} | | | |
| 312142 | EOS12073 | AW298359 | Hs.221069 | ESTs | 1.1 |
| 315779 | EOS15710 | AW015736 | Hs.211378 | ESTs | 1.1 |
| 318596 | EOS18527 | AI470235 | Hs.172698 | EST | 1.1 |
| 335701 | EOS35632 | CH22_3062FG_599_1_LINK_EM:AC005500.GENSCAN.490-2 | | | 1.1 |
| | | | | CH22_FGENES.599_1 | |
| 319395 | EOS19326 | AW062570 | Hs.13809 | ESTs | 1.1 |
| 304236 | EOS04167 | W93278 | | EST singleton (not in UniGene) with exon hit | 1.1 |
| 307264 | EOS07195 | AI202211 | | EST singleton (not in UniGene) with exon hit | 1.1 |
| 334066 | EOS33997 | CH22_1344FG_327_21_LINK_EM:AC005500.GENSCAN.181-23 | | | 1.1 |
| | | | | CH22_FGENES.327_21 | |
| 327042 | EOS26973 | c21_hs gi\|6531965\|ref\|gn 18 − 1380806 1381443 ex 1 5 CDSl 30.85 638 943 | | | 1.1 |
| | | | | CH.21_hs gi\|6531965 | |
| 326025 | EOS25956 | c17_hs gi\|5867176\|ref\|gn 1 + 70854 70915 ex 6 8 CDSi -1.46 62 127 | | | 1.1 |
| | | | | CH.17_hs gi\|5867176 | |
| 325609 | EOS25540 | c14_hs gi\|5866996\|ref\|gn 28 − 981751 981849 ex 1 10 CDSl 1.46 99 101 | | | 1.1 |
| | | | | CH.14_hs gi\|5866996 | |
| 319983 | EOS19914 | T81429 | | EST cluster (not in UniGene) | 1.1 |
| 334298 | EOS34229 | CH22_1589FG_372_4_LINK_EM:AC005500.GENSCAN.232-5 | | | 1.1 |
| | | | | CH22_FGENES.372_4 | |
| 323203 | EOS23134 | AA203135 | Hs.130186 | ESTs | 1.1 |
| 305700 | EOS05631 | AA815428 | | EST singleton (not in UniGene) with exon hit | 1.1 |
| 313304 | EOS13235 | AI334078 | Hs.152438 | ESTs | 1.1 |
| 310716 | EOS10647 | AI589618 | Hs.192413 | ESTs | 1.1 |
| 327049 | EOS26980 | c21_hs gi\|6531965\|ref\|gn 24 − 1924026 1924110 ex 2 6 CDSi 9.43 85 1012 | | | 1.1 |
| | | | | CH.21_hs gi\|6531965 | |
| 313749 | EOS13680 | AW450376 | Hs.130803 | ESTs | 1.1 |
| 307041 | EOS06972 | AI144243 | | EST singleton (not in UniGene) with exon hit | 1.1 |
| 322394 | EOS22325 | AF077208 | | EST cluster (not in UniGene) | 1.1 |
| 326416 | EOS26347 | c19_hs gi\|5867362\|ref\|gn 3 − 45283 45375 ex 3 3 CDSf 5.65 93 923 | | | 1.1 |
| | | | | CH.19_hs gi\|5867362 | |
| 333947 | EOS33878 | CH22_1221FG_303_1_LINK_EM:AC005500.GENSCAN.162-5 | | | 1.1 |
| | | | | CH22_FGENES.303_1 | |
| 324609 | EOS24540 | AW299534 | | EST cluster (not in UniGene) | 1.1 |
| 330057 | EOS29988 | c17_p2 gi\|6478962\|gb\|A gn 3 + 75145 75287 ex 3 3 CDSl -2.56 143 150 | | | 1.1 |
| | | | | CH.17_p2 gi\|6478962 | |
| 337603 | EOS37534 | CH22_5896FG_LINK_C20H12.GENSCAN.16-2 | | | 1.1 |
| | | | | CH22_C20H12.GENSCAN.16-2 | |
| 332913 | EOS32844 | CH22_134FG_36_18_LINK_C20H12.GENSCAN.28-17 | | | 1.1 |
| | | | | CH22_FGENES.36_18 | |
| 310026 | EOS09957 | T24895 | Hs.100691 | ESTs | 1.1 |
| 330153 | EOS30084 | c21_p2 gi\|4325335\|gb\|A gn 2 + 146951 147475 ex 2 2 CDSl 25.45 525 233 | | | 1.1 |
| | | | | CH.21_p2 gi\|4325335 | |
| 334118 | EOS34049 | CH22_1396FG_330_19_LINK_EM:AC005500.GENSCAN.185-20 | | | 1.1 |
| | | | | CH22_FGENES.330_19 | |
| 324795 | EOS24726 | AI494481 | Hs.141579 | ESTs | 1.1 |
| 332530 | EOS32461 | M31682 | Hs.1735 | inhibin; beta B (activin AB beta polypeptide) | 1.1 |
| 332048 | EOS31979 | AA496019 | Hs.201591 | ESTs | 1.1 |
| 334532 | EOS34463 | CH22_1834FG_402_13_LINK_EM:AC005500.GENSCAN.266-13 | | | 1.1 |
| | | | | CH22_FGENES.402_13 | |
| 329762 | EOS29693 | c14_p2 gi\|6048280\|emb\|gn 3 + 127744 127878 ex 2 4 CDSi 11.66 135 1054 | | | 1.1 |
| | | | | CH.14_p2 gi\|6048280 | |
| 332909 | EOS32840 | CH22_130FG_36_13_LINK_C20H12.GENSCAN.28-10 | | | 1.1 |
| | | | | CH22_FGENES.36_13 | |
| 321253 | EOS21184 | AI699484 | | EST cluster (not in UniGene) | 1.1 |
| 336572 | EOS36503 | CH22_4007FG_843_12_LINK_DJ579N16.GENSCAN.15-13 | | | 1.1 |
| | | | | CH22_FGENES.843_12 | |
| 328768 | EOS28699 | c_7_hs gi\|6017031\|ref\|gn 5 − 223741 224238 ex 1 1 CDSo 30.00 498 5285 | | | 1.1 |
| | | | | CH.07_hs gi\|6017031 | |
| 334335 | EOS34266 | CH22_1627FG_375_12_LINK_EM:AC005500.GENSCAN.235-12 | | | 1.1 |
| | | | | CH22_FGENES.375_12 | |
| 334063 | EOS33994 | CH22_1341FG_327_17_LINK_EM:AC005500.GENSCAN.181-20 | | | 1.1 |
| | | | | CH22_FGENES.327_17 | |
| 333011 | EOS32942 | CH22_235FG_61_3_LINK_EM:AC000097.GENSCAN.23-3 | | | 1.1 |
| | | | | CH22_FGENES.61_3 | |
| 304677 | EOS04608 | AA548071 | | EST singleton (not in UniGene) with exon hit | 1.1 |
| 313948 | EOS13879 | AW452823 | Hs.135268 | ESTs | 1.1 |
| 334358 | EOS34289 | CH22_1652FG_378_1_LINK_EM:AC005500.GENSCAN.239-1 | | | 1.1 |
| | | | | CH22_FGENES.378_1 | |
| 328479 | EOS28410 | c_7_hs gi\|5868449\|ref\|gn 1 − 331 560 ex 1 31 CDSi 18.51 230 2100 | | | 1.1 |
| | | | | CH.07_hs gi\|5868449 | |
| 335813 | EOS35744 | CH22_3185FG_618_1_LINK_EM:AC005500.GENSCAN.510-1 | | | 1.1 |
| | | | | CH22_FGENES.618_1 | |
| 312430 | EOS12361 | AW139117 | Hs.117494 | ESTs | 1.1 |
| 324783 | EOS24714 | AA640770 | | EST cluster (not in UniGene) | 1.1 |
| 337776 | EOS37707 | CH22_6132FG_LINK_EM:AC000097.GENSCAN.119-18 | | | 1.1 |
| | | | | CH22_EM:AC000097.GENSCAN.119-18 | |
| 327205 | EOS27136 | c_1_hs gi\|5867447\|ref\|gn 5 + 167335 167576 ex 9 9 CDSl 15.50 242 259 | | | 1.1 |
| | | | | CH.01_hs gi\|5867447 | |
| 315198 | EOS15129 | AI741506 | Hs.186753 | ESTs; Weakly similar to !!!! ALU SUBFAMILY J WARNING ENTRY !!!! [H. sapiens] | 1.1 |
| 336135 | EOS36066 | CH22_3525FG_704_3_LINK_DA59H18.GENSCAN.9-5 | | | 1.1 |
| | | | | CH22_FGENES.704_3 | |
| 318558 | EOS18489 | AW402677 | Hs.90372 | ESTs | 1.1 |
| 328152 | EOS28083 | c_6_hs gi\|5868060\|ref\|gn 1 − 73981 74203 ex 1 8 CDSl 31.69 223 3411 | | | 1.1 |
| | | | | CH.06_hs gi\|5868060 | |
| 330211 | EOS30142 | c_5_p2 gi\|6013592\|gb\|A gn 1 + 59158 59215 ex 2 4 CDSi 4.20 58 184 | | | 1.1 |
| | | | | CH.05_p2 gi\|6013592 | |
| 339280 | EOS39211 | CH22_8234FG_LINK_BA354I12.GENSCAN.14-12 | | | 1.1 |
| | | | | CH22_BA354I12.GENSCAN.14-12 | |
| 332045 | EOS31976 | AA491253 | Hs.155045 | bromodomain adjacent to zinc finger domain; 2A | 1.1 |
| 313597 | EOS13528 | AW162263 | Hs.249990 | ESTs | 1.1 |
| 329503 | EOS29434 | c10_p2 gi\|3983517\|gb\|U gn 2 − 1801 1937 ex 1 4 CDSl 4.33 137 101 | | | 1.1 |
| | | | | CH.10_p2 gi\|3983517 | |
| 333488 | EOS33419 | CH22_740FG_167_3_LINK_EM:AC005500.GENSCAN.46-10 | | | 1.1 |
| | | | | CH22_FGENES.167_3 | |
| 311960 | EOS11891 | AW440133 | Hs.189690 | ESTs | 1.1 |
| 320590 | EOS20521 | U67058 | Hs.168102 | Human proteinase activated receptor-2 mRNA; 3′UTR | 1.1 |
| 334047 | EOS33978 | CH22_1325FG_326_5_LINK_EM:AC005500.GENSCAN.175-5 | | | 1.1 |
| | | | | CH22_FGENES.326_5 | |
| 304782 | EOS04713 | AA582081 | | EST singleton (not in UniGene) with exon hit | 1.1 |
| 324231 | EOS24162 | W60827 | | EST cluster (not in UniGene) | 1.1 |
| 327212 | EOS27143 | c_1_hs gi\|5867463\|ref\|gn 1 − 42308 42424 ex 5 13 CDSi 6.58 117 325 | | | 1.1 |
| | | | | CH.01_hs gi\|5867463 | |
| 335857 | EOS35788 | CH22_3232FG_629_1_LINK_EM:AC005500.GENSCAN.519-1 | | | 1.1 |
| | | | | CH22_FGENES.629_1 | |
| 317775 | EOS17706 | AA974603 | Hs.181123 | ESTs | 1.1 |
| 331053 | EOS30984 | N70242 | Hs.183146 | ESTs | 1.1 |
| 335940 | EOS35871 | CH22_3318FG_646_13_LINK_DJ246D7.GENSCAN.1-12 | | | 1.1 |
| | | | | CH22_FGENES.646_13 | |
| 322568 | EOS22499 | W87342 | Hs.209652 | ESTs | 1.1 |
| 314091 | EOS14022 | AI253112 | Hs.133540 | ESTs | 1.1 |
| 313570 | EOS13501 | AA041455 | Hs.209312 | ESTs | 1.1 |
| 300967 | EOS00898 | AA565209 | Hs.190216 | ESTs | 1.1 |
| 314544 | EOS14475 | AA399018 | Hs.250835 | ESTs | 1.1 |
| 328321 | EOS28252 | c_7_hs gi\|5868373\|ref\|gn 7 − 1029614 1029673 ex 1 3 CDSl-2.40 60 448 | | | 1.1 |
| | | | | CH.07_hs gi\|5868373 | |
| 310979 | EOS10910 | AW445166 | Hs.170802 | ESTs | 1.1 |
| 310730 | EOS10661 | AI939421 | Hs.160900 | ESTs | 1.1 |
| 318471 | EOS18402 | AW137725 | Hs.146874 | ESTs | 1.1 |
| 315533 | EOS15464 | AW206191 | Hs.152774 | ESTs | 1.1 |
| 325751 | EOS25682 | c14_hs gi\|6682474\|ref\|gn 4 + 130437 130520 ex 6 7 CDSi 0.22 84 1666 | | | 1.1 |
| | | | | CH.14_hs gi\|6682474 | |
| 318780 | EOS18711 | R90906 | Hs.113307 | ESTs | 1.1 |
| 313271 | EOS13202 | AW444819 | Hs.144851 | ESTs; Weakly similar to C09F5.2 [C.elegans] | 1.1 |
| 304546 | EOS04477 | AA486074 | | EST singleton (not in UniGene) with exon hit | 1.1 |
| 330618 | EOS30549 | X55990 | Hs.73839 | ribonuclease; RNase A family; 3 (eosinophil cationic protein) | 1.1 |
| 332931 | EOS32862 | CH22_152FG_38_5_LINK_C20H12.GENSCAN.29-5 | | | 1.1 |
| | | | | CH22_FGENES.38_5 | |
| 336602 | EOS36533 | CH22_4047FG_372_4_LINK_EM:AC005500.GENSCAN.232-4 | | | 1.1 |
| | | | | CH22_FGENES.372_4 | |
| 311185 | EOS11116 | AI638294 | Hs.224665 | ESTs | 1.1 |
| 337585 | EOS37516 | CH22_5873FG_LINK_C20H12.GENSCAN.5-3 | | | 1.1 |
| | | | | CH22_C20H12.GENSCAN.5-3 | |
| 310249 | EOS10180 | AW071751 | HS.13179 | ESTs; Moderately similar to !!!! ALU SUBFAMILY SQ WARNING ENTRY !!!! | 1.1 |
| | | | | [H. sapiens] | |
| 314578 | EOS14509 | AA410183 | Hs.137475 | ESTs | 1.1 |
| 310750 | EOS10681 | AI373163 | Hs.170333 | ESTs | 1.1 |
| 333968 | EOS33899 | CH22_1245FG_307_4_LINK_EM:AC005500.GENSCAN.165-5 | | | 1.1 |
| | | | | CH22_FGENES.307_4 | |
| 316133 | EOS16064 | AI187742 | Hs.125562 | ESTs | 1.1 |
| 308337 | EOS08268 | AI608947 | | EST singleton (not in UniGene) with exon hit | 1.1 |
| 326160 | EOS26091 | c17_hs gi\|5867254\|ref\|gn 6 − 112000 112137 ex 2 4 CDSi 8.01 138 1952 | | | 1.1 |
| | | | | CH.17_hs gi\|5867254 | |
| 336023 | EOS35954 | CH22_3406FG_669_12_LINK_DJ32\|10.GENSCAN.9-17 | | | 1.1 |
| | | | | CH22_FGENES.669_12 | |
| 323479 | EOS23410 | AA278246 | | EST cluster (not in UniGene) | 1.1 |
| 336090 | EOS36021 | CH22_3477FG_689_2_LINK_DJ32\|10.GENSCAN.23-20 | | | 1.1 |
| | | | | CH22_FGENES.689_2 | |
| 311192 | EOS11123 | AW237220 | Hs.211130 | ESTs | 1.1 |
| 335081 | EOS35012 | CH22_2409FG_488_4_LINK_EM:AC005500.GENSCAN.384-6 | | | 1.1 |
| | | | | CH22_FGENES.488_4 | |
| 309519 | EOS09450 | AW148940 | Hs.248647 | EST | 1.1 |
| 321172 | EOS21103 | H49160 | Hs.133472 | ESTs | 1.1 |
| 301976 | EOS01907 | T97905 | | EST cluster (not in UniGene) with exon hit | 1.1 |
| 323012 | EOS22943 | AI832201 | Hs.211469 | ESTs | 1.1 |
| 319528 | EOS19459 | R08673 | Hs.177514 | ESTs | 1.1 |
| 329838 | EOS29769 | c14_p2 gi\|6672062\|emb\|gn 2 + 33990 34098 ex 3 4 CDSi 9.11 109 2222 | | | 1.1 |
| | | | | CH.14_p2 gi\|6672062 | |
| 302623 | EOS02554 | AB019571 | | EST cluster (not in UniGene) with exon hit | 1.1 |
| 334433 | EOS34364 | CH22_1731FG_385_8_LINK_EM:AC005500.GENSCAN.249-6 | | | 1.1 |
| | | | | CH22_FGENES.385_8 | |
| 304747 | EOS04678 | AA577816 | | EST singleton (not in UniGene) with exon hit | 1.1 |
| 333270 | EOS33201 | CH22_513FG_121_1_LINK_EM:AC005500.GENSCAN.4-11 | | | 1.1 |
| | | | | CH22_FGENES.121_1 | |
| 307054 | EOS06985 | AI148181 | Hs.176835 | EST | 1.1 |
| 320764 | EOS20695 | R73070 | Hs.246927 | ESTs | 1.1 |
| 321523 | EOS21454 | H78472 | Hs.191325 | ESTs; Weakly similar to cDNA EST yk414c9.3 comes from this gene [C.elegans] | 1.1 |
| 322114 | EOS22045 | AA643791 | Hs.191740 | ESTs | 1.1 |
| 303582 | EOS03513 | AA377444 | | EST cluster (not in UniGene) with exon hit | 1.1 |
| 322924 | EOS22855 | AA669253 | Hs.193971 | ESTs | 1.1 |
| 311179 | EOS11110 | AI880843 | Hs.223333 | ESTs | 1.1 |
| 318601 | EOS18532 | T39921 | | EST cluster (not in UniGene) | 1.1 |
| 309791 | EOS09722 | AW276176 | Hs.73742 | ribosomal protein; large; P0 | 1.1 |
| 333882 | EOS33813 | CH22_1153FG_292_4_LINK_EM:AC005500.GENSCAN.150-4 | | | 1.1 |
| | | | | CH22_FGENES.292_4 | |
| 337645 | EOS37576 | CH22_5960FG_LINK_EM:AC000097.GENSCAN.10-8 | | | 1.1 |
| | | | | CH22_EM:AC000097.GENSCAN.10-8 | |
| 335623 | EOS35554 | CH22_2983FG_584_2_LINK_EM:AC005500.GENSCAN.478-2 | | | 1.1 |
| | | | | CH22_FGENES.584_2 | |
| 314745 | EOS14676 | AA564489 | Hs.137526 | ESTs | 1.1 |
| 330790 | EOS30721 | T48536 | Hs.105807 | ESTs | 1.1 |
| 332071 | EOS32002 | AA598594 | Hs.112475 | ESTs | 1.1 |
| 312005 | EOS11936 | T78450 | Hs.13941 | ESTs | 1.1 |
| 330694 | EOS30625 | AA019806 | Hs.108447 | spinocerebellar ataxia 7 (olivopontocerebellar atrophy with retinal degeneration) | 1.1 |
| 330739 | EOS30670 | AA293477 | Hs.227591 | ESTs | 1.1 |
| 303042 | EOS02973 | AF129532 | | EST cluster (not in UniGene) with exon hit | 1.1 |
| 323091 | EOS23022 | AW014094 | Hs.210761 | ESTs | 1.1 |
| 328820 | EOS28751 | c_7_hs gi\|5868330\|ref\|gn 1 + 90446 90602 ex 3 4 CDSi 10.20 157 5634 | | | 1.1 |
| | | | | CH.07_hs gi\|5868330 | |
| 300472 | EOS00403 | T90622 | Hs.82609 | hydroxymethylbilane synthase | 1.1 |
| 310645 | EOS10576 | AI420742 | Hs.163502 | ESTs | 1.1 |
| 332238 | EOS32169 | N53480 | Hs.108622 | ESTs | 1.1 |
| 300966 | EOS00897 | AA564740 | Hs.258401 | ESTs | 1.1 |
| 330437 | EOS30368 | HG2730-HT2827 | | Fibrinogen, A Alpha Polypeptide, Alt. Splice 2, E | 1.1 |
| 302292 | EOS02223 | AF067797 | | EST cluster (not in UniGene) with exon hit | 1.1 |
| 330138 | EOS30069 | c21_p2 gi\|4210430\|emb\|gn 1 − 22334 22460 ex 3 3 CDSf 16.56 127 105 | | | 1.1 |
| | | | | CH.21_p2 gi\|4210430 | |
| 332952 | EOS32883 | CH22_176FG_48_8_LINK_EM:AC000097.GENSCAN.2-4 | | | 1.1 |
| | | | | CH22_FGENES.48_8 | |
| 319901 | EOS19832 | T77136 | Hs.8765 | RNA helicase-related protein | 1.1 |
| 321166 | EOS21097 | AA411263 | Hs.128783 | ESTs | 1.1 |
| 336227 | EOS36158 | CH22_3625FG_730_2_LINK_DA59H18.GENSCAN.36-2 | | | 1.1 |
| | | | | CH22_FGENES.730_2 | |
| 302332 | EOS02263 | AI833168 | Hs.184507 | Homo sapiens Chromosome 16 BAC clone CIT987SK-A-328A3 | 1.1 |
| 313800 | EOS13731 | AW296132 | Hs.166674 | ESTs | 1.1 |
| 339356 | EOS39287 | CH22_8326FG_LINK_BA354\|12.GENSCAN.31-1 | | | 1.1 |
| | | | | CH22_BA354\|12.GENSCAN.31-1 | |
| 324512 | EOS24443 | AW502125 | | EST cluster (not in UniGene) | 1.1 |
| 319235 | EOS19166 | F11330 | Hs.177633 | ESTs | 1.1 |
| 320352 | EOS20283 | Y13323 | Hs.145296 | disintegrin protease | 1.1 |
| 338316 | EOS38247 | CH22_6944FG_LINK_EM:AC005500.GENSCAN.304-2 | | | 1.1 |
| | | | | CH22_EM:AC005500.GENSCAN.304-2 | |
| 333964 | EOS33895 | CH22_1241FG_305_2_LINK_EM:AC005500.GENSCAN.164-2 | | | 1.1 |
| | | | | CH22_FGENES.305_2 | |
| 312758 | EOS12689 | AA721107 | Hs.202604 | ESTs | 1.1 |
| 338178 | EOS38109 | CH22_6726FG_LINK_EM:AC005500.GENSCAN.219-6 | | | 1.1 |
| | | | | CH22_EM:AC005500.GENSCAN.219-6 | |
| 315199 | EOS15130 | AA877996 | Hs.125376 | ESTs | 1.1 |
| 312321 | EOS12252 | R66210 | Hs.186937 | ESTs | 1.1 |
| 338765 | EOS38696 | CH22_7588FG_LINK_EM:AC005500.GENSCAN.518-1 | | | 1.1 |
| | | | | CH22_EM:AC005500.GENSCAN.518-1 | |
| 330547 | EOS30478 | U32989 | Hs.183671 | tryptophan 2;3-dioxygenase | 1.1 |
| 315368 | EOS15299 | AW291563 | Hs.152495 | ESTs | 1.1 |
| 328691 | EOS28622 | c_7_hs gi\|6588001\|ref\|gn 7 − 579598 579664 ex 2 3 CDSi 12.78 67 4326 | | | 1.1 |
| | | | | CH.07_hs gi\|6588001 | |
| 329179 | EOS29110 | c_x_hs gi\|5868704\|ref\|gn 2 + 181639 181815 ex 3 4 CDSi 0.32 177 1939 | | | 1.1 |
| | | | | CH.X_hs gi\|5868704 | |
| 327072 | EOS27003 | c21_hs gi\|6531965\|ref\|gn 55 − 3796429 3797197 ex 4 4 CDSf 9.33 769 1270 | | | 1.1 |
| | | | | CH.21_hs gi\|6531965 | |
| 312056 | EOS11987 | T83748 | Hs.189712 | ESTs | 1.1 |
| 339128 | EOS39059 | CH22_8046FG_LINK_DA59H18.GENSCAN.55-2 | | | 1.1 |
| | | | | CH22_DA59H18.GENSCAN.55-2 | |
| 307646 | EOS07577 | AI302236 | | EST singleton (not in UniGene) with exon hit | 1.1 |
| 319198 | EOS19129 | F07354 | | EST cluster (not in UniGene) | 1.1 |
| 338556 | EOS38487 | CH22_7283FG_LlNK_EM:AC005500.GENSCAN.417-8 | | | 1.1 |
| | | | | CH22_EM:AC005500.GENSCAN.417-8 | |
| 306143 | EOS06074 | AA916314 | | EST singleton (not in UniGene) with exon hit | 1.1 |
| 332384 | EOS32315 | M11433 | Hs.101850 | retinol-binding protein 1; cellular | 1.1 |
| 325100 | EOS25031 | T10265 | Hs.116122 | ESTs; Weakly similar to coded for by C. elegans cDNA yk30b3.5 [C. elegans] | 1.1 |
| 309839 | EOS09770 | AW298076 | | EST singleton (not in UniGene) with exon hit | 1.1 |
| 312180 | EOS12111 | AI248285 | Hs.118348 | ESTs | 1.1 |
| 330385 | EOS30316 | AA449749 | Hs.31386 | ESTs; Highly similar to secreted apoptosis related protein 1 [H. sapiens] | 1.1 |
| 315882 | EOS15813 | AI831297 | Hs.123310 | ESTs | 1.1 |
| 325843 | EOS25774 | c16_hs gi\|6552453\|ref\|gn 1 − 7126 7232 ex 1 3 CDSi 1.87 107 182 | | | 1.1 |
| | | | | CH.16_hs gi\|6552453 | |
| 330783 | EOS30714 | D60050 | Hs.34812 | ESTs | 1.1 |
| 317224 | EOS17155 | D56760 | Hs.8122 | ESTs | 1.1 |
| 316042 | EOS15973 | AW297979 | Hs.170698 | ESTs | 1.1 |
| 333524 | EOS33455 | CH22_781FG_175_10_LINK_EM:AC005500.GENSCAN.53-15 | | | 1.1 |
| | | | | CH22_FGENES.175_10 | |
| 302357 | EOS02288 | X03178 | Hs.198246 | group-specific component (vitamin D binding protein) | 1.1 |
| 309830 | EOS09761 | AW294725 | | EST singleton (not in UniGene) with exon hit | 1.1 |
| 321489 | EOS21420 | AW392474 | Hs.172759 | ESTs; Moderately similar to !!!! ALU SUBFAMILY SQ WARNING ENTRY !!!! | 1.1 |
| | | | | [H. sapiens] | |
| 312304 | EOS12235 | AA491949 | Hs.183359 | ESTs | 1.1 |
| 322026 | EOS21957 | AA233527 | Hs.213289 | low density lipoprotein receptor (familial hypercholesterolemia) | 1.1 |

PKey Primekey(unique probeset identifier)
Ex. Accn. Exemplar accession number
Probeset Eos Code number
Unigene# Unigene number

Table 2 provides the nucleic acid and protein sequence of the CBF9 gene as well as the Unigene and Exemplar accession numbers for CBF9.

TABLE 2

CBF9 DNA and Protein Sequences

CBF9 DNA sequence

| | |
|---|---|
| Gene name: | ESTs |
| Unigene number: | Hs.157601 |
| Probeset Accession #: | W07459 |
| Nucleic Acid Accession #: | AC005383 |
| Coding Sequence: | 328-2751 (underlined sequences correspond to start and |
| | stop codons) |

| | |
|---|---|
| 1 11 21 31 41 51 | |
| \| \| \| \| \| \| | |
| GACAGTGTTC GCGGCTGCAC CGCTCGGAGG CTGGGTGACC CGCGTAGAAG TGAAGTACTT | 60 |
| TTTTATTTGC AGACCTGGGC CGATGCCGCT TTAAAAAACG CGAGGGGCTC TATGCACCTC | 120 |
| CCTGGCGGTA GTTCCTCCGA CCTCAGCCGG GTCGGGTCGT GCCGCCCTCT CCCAGGAGAG | 180 |
| ACAAACAGGT GTCCCACGTG GCAGCCGCGC CCCGGGCGCC CCTCCTGTGA TCCCGTAGCG | 240 |
| CCCCCTGGCC CGAGCCGCGC CCGGGTCTGT GAGTAGAGCC GCCCGGGCAC CGAGCGCTGG | 300 |
| TCGCCGCTCT CCTTCCGTTA TATCAACATG CCCCCTTTCC TGTTGCTGGA GGCCGTCTGT | 360 |
| GTTTTCCTGT TTTCCAGAGT GCCCCCATCT CTCCCTCTCC AGGAAGTCCA TGTAAGCAAA | 420 |
| GAAACCATCG GGAAGATTTC AGCTGCCAGC AAAATGATGT GGTGCTCGGC TGCAGTGGAC | 480 |
| ATCATGTTTC TGTTAGATGG GTCTAACAGC GTCGGGAAAG GGAGCTTTGA AAGGTCCAAG | 540 |
| CACTTTGCCA TCACAGTCTG TGACGGTCTG GACATCAGCC CCGAGAGGGT CAGAGTGGGA | 600 |
| GCATTCCAGT TCAGTTCCAC TCCTCATCTG GAATTCCCCT TGGATTCATT TTCAACCCAA | 660 |
| CAGGAAGTGA AGGCAAGAAT CAAGAGGATG GTTTTCAAAG GAGGGCGCAC GGAGACGGAA | 720 |
| CTTGCTCTGA AATACCTTCT GCACAGAGGG TTGCCTGGAG GCAGAAATGC TTCTGTGCCC | 780 |
| CAGATCCTCA TCATCGTCAC TGATGGGAAG TCCCAGGGGG ATGTGGCACT GCCATCCAAG | 840 |
| CAGCTGAAGG AAAGGGGTGT CACTGTGTTT GCTGTGGGGG TCAGGTTTCC CAGGTGGGAG | 900 |
| GAGCTGCATG CACTGGCCAG CGAGCCTAGA GGGCAGCACG TGCTGTTGGC TGAGCAGGTG | 960 |
| GAGGATGCCA CCAACGGCCT CTTCAGCACC CTCAGCAGCT CGGCCATCTG CTCCAGCGCC | 1020 |
| ACGCCAGACT GCAGGGTCGA GGCTCACCCC TGTGAGCACA GGACGCTGGA GATGGTCCGG | 1080 |
| GAGTTCGCTG GCAATGCCCC ATGCTGGAGA GGATCGCGGC GGACCCTTGC GGTGCTGGCT | 1140 |
| GCACACTGTC CCTTCTACAG CTGGAAGAGA GTGTTCCTAA CCCACCCTGC CACCTGCTAC | 1200 |
| AGGACCACCT GCCCAGGCCC CTGTGACTCG CAGCCCTGCC AGAATGGAGG CACATGTGTT | 1260 |
| CCAGAAGGAC TGGACGGCTA CCAGTGCCTC TGCCCGCTGG CCTTTGGAGG GGAGGCTAAC | 1320 |
| TGTGCCCTGA AGCTGAGCCT GGAATGCAGG GTCGACCTCC TCTTCCTGCT GGACAGCTCT | 1380 |
| GCGGGCACCA CTCTGGACGG CTTCCTGCGG GCCAAAGTCT TCGTGAAGCG GTTTGTGCGG | 1440 |
| GCCGTGCTGA GCGAGGACTC TCGGGCCCGA GTGGGTGTGG CCACATACAG CAGGGAGCTG | 1500 |
| CTGGTGGCGG TGCCTGTGGG GGAGTACCAG GATGTGCCTG ACCTGGTCTG GAGCCTCGAT | 1560 |
| GGCATTCCCT TCCGTGGTGG CCCCACCCTG ACGGGCAGTG CCTTGCGGCA GGCGGCAGAG | 1620 |
| CGTGGCTTCG GGAGCGCCAC CAGGACAGGC CAGGACCGGC CACGTAGAGT GGTGGTTTTG | 1680 |
| CTCACTGAGT CACACTCCGA GGATGAGGTT GCGGGCCCAG CGCGTCACGC AAGGGCGCGA | 1740 |
| GAGCTGCTCC TGCTGGGTGT AGGCAGTGAG GCCGTGCGGG CAGAGCTGGA GGAGATCACA | 1800 |
| GGCAGCCCAA AGCATGTGAT GGTCTACTCG GATCCTCAGG ATCTGTTCAA CCAAATCCCT | 1860 |
| GAGCTGCAGG GGAAGCTGTG CAGCCGGCAG CGGCCAGGGT GCCGGACACA AGCCCTGGAC | 1920 |
| CTCGTCTTCA TGTTGGACAC CTCTGCCTCA GTACGGCCCG AGAATTTTGC TCAGATGCAG | 1980 |
| AGCTTTGTGA GAAGCTGTGC CCTCCAGTTT GAGGTGAACC CTGACGTGAC ACAGGTCGGC | 2040 |
| CTGGTGGTGT ATGGCAGCCA GGTGCAGACT GCCTTCGGGC TGGACACCAA ACCCACCCGG | 2100 |
| GCTGCGATGC TGCGGGCCAT TAGCCAGGCC CCCTACCTAG GTGGOGTGGG CTCAGCCGGC | 2160 |
| ACCGCCCTGC TGCACATCTA TGACAAAGTG ATGACCGTCC AGAGGGGTGC CCGGCCTGGT | 2220 |
| GTCCCCAAAG CTGTGGTGGT GCTCACAGGC GGGAGAGGCG CAGAGGATGC AGCCGTTCCT | 2280 |
| GCCCAGAAGC TGAGGAACAA TGGCATCTCT GTCTTGGTCG TGGGCGTGGG GCCTGTCCTA | 2340 |
| AGTGAGGGTC TGCGGAGGCT TGCAGGTCCC CGGGATTCCC TGATCCACGT GGCAGCTTAC | 2400 |
| GCCGACCTGC GGTACCACCA GGACGTGCTC ATTGAGTGGC TGTGTGGAGA AGCCAAGCAG | 2460 |
| CCAGTCAACC TCTGCAAACC CAGCCCGTGC ATGAATGAGG GCAGCTGCGT CCTGCAGAAT | 2520 |
| GGGAGCTACC GCTGCAAGTG TCGGGATGGC TGGGAGGGCC CCCACTGCGA GAACCGTGAG | 2580 |
| TGGAGCTCTT GCTCTGTATG TGTGAGCCAG GGATGGATTC TTGAGACGCC CCTGAGGCAC | 2640 |
| ATGGCTCCCG TGCAGGAGGG CAGCAGCCGT ACCCCTCCCA GCAACTACAG AGAAGGCCTG | 2700 |
| GGCACTGAAA TGGTGCCTAC CTTCTGGAAT GTCTGTGCCC CAGGTCCTTA GAATGTCTGC | 2760 |
| TTCCCGCCGT GGCCAGGACC ACTATTCTCA CTGAGGGAGG AGGATGTCCC AACTGCAGCC | 2820 |
| ATGCTGCTTA GAGACAAGAA AGCAGCTGAT GTCACCCACA AACGATGTTG TTGAAAAGTT | 2880 |
| TTGATGTGTA AGTAAATACC CACTTTCTGT ACCTGCTGTG CCTTGTTGAG GCTATGTCAT | 2940 |
| CTGCCACCTT TCCCTTGAGG ATAAACAAGG GGTCCTGAAG ACTTAAATTT AGCGGCCTGA | 3000 |
| CGTTCCTTTG CACACAATCA ATGCTCGCCA GAATGTTGTT GACACAGTAA TGCCCAGCAG | 3060 |
| AGGCCTTTAC TAGAGCATCC TTTGGACGGC GAAGGCCACG GCCTTTCAAG ATGGAAAGCA | 3120 |
| GCAGCTTTTC CACTTCCCCA GAGACATTCT GGATGCATTT GCATTGAGTC TGAAAGGGGG | 3180 |
| CTTGAGGGAC GTTTGTGACT TCTTGGCGAC TGCCTTTTGT GTGTGGAAGA GACTTGGAAA | 3240 |
| GGTCTCAGAC TGAATGTGAC CAATTAACCA GCTTGGTTGA TGATGGGGGA GGGGCTGAGT | 3300 |
| TGTGCATGGG CCCAGGTCTG GAGGGCCACG TAAAATCGTT CTGAGTCGTG AGCAGTGTCC | 3360 |
| ACCTTGAAGG TCTTC | |

CBF9 Protein sequence

| | |
|---|---|
| Gene name: | ESTs |
| Imigene number: | Hs.157601 |

Protein Accession #: none found

| | |
|---|---|
| Signal sequence: | 1-17 |
| Transmembrane domains: | none found |
| VGW domains: | 49-223; 341-518; 529-706 |
| EGF domains: | 298-333; 715-748 |
| Cellular Localization: | plasma membrane |

| | |
|---|---|
| 1 11 21 31 41 51 | |
| \| \| \| \| \| \| | |
| MPPFLLLEAV CVFLFSRVPP SLPLQEVHVS KETIGKISAA SKMMWCSAAV DIMFLLDGSN | 60 |
| SVGKGSFERS KHFAITVCDG LDISPERVRV GAFQFSSTPH LEFPLDSFST QQEVKARIKR | 120 |
| MVFKGGRTET ELALKYLLHR GLPGGRNASV PQILIIVTDG KSQGDVALPS KQLKERGVTV | 180 |
| FAVGVRFPRW EELHALASEP RGQHVLLAEQ VEDATNGLFS TLSSSAICSS ATPDCRVEAH | 240 |
| PCEHRTLEMV REFAGNAPCW RGSRRTLAVL AAHCPFYSWK RVFLTHPATC YRTTCPGPCD | 300 |
| SQPCQNGGTC VPEGLDGYQC LCPLAFGGEA NCALKLSLEC RVDLLFLLDS SAGTTLDGFL | 360 |
| RAKVFVKRFV RAVLSEDSRA RVGVATYSRE LLVAVPVGEY QDVPDLVWSL DGIPFRGGPT | 420 |
| LTGSALRQAA ERGFGSATRT GQDRPRRVVV LLTESHSEDE VAGPARHARA RELLLLGVGS | 480 |
| EAVRAELEEI TGSPKHVMVY SDPQDLFNQI PELQGKLCSR QRPGCRTQAL DLVFMLDTSA | 540 |
| SVGPENFAQM QSFVRSCALQ FEVNPDVTQV GLVVYGSQVQ TAFGLDTXPT RAAMLRAISQ | 600 |
| APYLGGVGSA GTALLHIYDK VMTVQRGARP GVPKAVVVLT GGRGAEDAAV PAQKLRNNGI | 660 |
| SVLVVGVGPV LSEGLRRLAG PRDSLIHVAA YADLRYHQDV LIEWLCGEAK QPVNLCKPSP | 720 |
| CMNEGSCVLQ NGSYRCKCRD GWEGPRCENR EWSSCSVCVS QGWILETPLR HMAPVQEGSS | 780 |
| RTPPSNYREG LGTEMVPTFW NVCAPGP | |

What is claimed is:

1. A method of screening drug candidates comprising:
a) providing a cell that expresses an expression profile gene selected from the group consisting of an expression profile gene set forth in Table 1 or Table 2 or fragment thereof;
b) adding a drug candidate to said cell; and
c) determining the effect of said drug candidate on the expression of said expression profile gene.

2. A method according to claim 1 wherein said determining comprises comparing the level of expression in the absence of said drug candidate to the level of expression in the presence of said drug candidate.

3. A method of screening for a bioactive agent capable of binding to a colorectal cancer modulator protein (colorectal cancer modulator protein), wherein said colorectal cancer modulator protein is encoded by a nucleic acid selected from the group consisting of a nucleic acid of Table 1 or Table 2 or a fragment thereof, said method comprising:
a) combining said colorectal cancer modulator protein and a candidate bioactive agent; and
b) determining the binding of said candidate agent to said colorectal cancer modulator protein.

4. A method for screening for a bioactive agent capable of modulating the activity of a colorectal cancer modulator protein, wherein said colorectal cancer modulator protein is encoded by a nucleic acid selected from the group consisting of a nucleic acid of Table 1 or Table 2 or a fragment thereof, said method comprising:
a) combining said colorectal cancer modulator protein and a candidate bioactive agent; and
b) determining the effect of said candidate agent on the bioactivity of said colorectal cancer modulator protein.

5. A method of evaluating the effect of a candidate colorectal cancer drug comprising:
a) administering said drug to a patient;
b) removing a cell sample from said patient; and
c) determining the expression of a gene selected from the group consisting of a nucleic acid of Table 1 or Table 2.

6. A method according to claim 5 further comprising comparing said expression profile to an expression profile of a healthy individual.

7. A method of diagnosing colorectal cancer comprising:
a) determining the expression of one or more genes selected from the group consisting of a nucleic acid of Table 1 or Table 2 or a fragment thereof or a polypeptide encoded thereby in a first tissue type of a first individual; and
b) comparing said expression of said gene(s) from a second normal tissue type from said first individual or a second unaffected individual;
wherein a difference in said expression indicates that the first individual has colorectal cancer.

8. A method for screening for a bioactive agent capable of interfering with the binding of a colorectal cancer modulator protein (colorectal cancer modulator protein) or a fragment thereof and an antibody which binds to said colorectal cancer modulator protein or fragment thereof, said method comprising:
a) combining a colorectal cancer modulator protein or fragment thereof, a candidate bioactive agent and an antibody which binds to said colorectal cancer modulator protein or fragment thereof; and
b) determining the binding of said colorectal cancer modulator protein or fragment thereof and said antibody.

9. A method for inhibiting the activity of a colorectal cancer modulator protein (colorectal cancer modulator protein), wherein said colorectal cancer modulator protein is encoded by a nucleic acid selected from the group consisting of a nucleic acid of Table 1 or Table 2 or a fragment thereof, said method comprising binding an inhibitor to said colorectal cancer modulator protein.

10. A method according to claim 9 wherein said inhibitor is an antibody.

11. A method of treating colorectal cancer comprising administering to a patient an inhibitor of a colorectal cancer modulator protein, wherein said colorectal cancer modulator protein is encoded by a nucleic acid selected from the group consisting of a nucleic acid of Table 1 or Table 2 or a fragment thereof.

12. A method according to claim 11 wherein said inhibitor is an antibody.

13. A method of neutralizing the effect of a colorectal cancer modulator protein, or a fragment thereof, comprising contacting an agent specific for said protein with said protein in an amount sufficient to effect neutralization.

14. A method for localizing a therapeutic moiety to colorectal cancer tissue comprising exposing said tissue to an antibody to a colorectal cancer modulator protein or fragment thereof conjugated to said therapeutic moiety.

15. The method of claim 14, wherein said therapeutic moiety is a cytotoxic agent.

16. The method of claim 14, wherein said therapeutic moiety is a radioisotope.

17. A method for inhibiting colorectal cancer in a cell, wherein said method comprises administering to a cell a composition comprising antisense molecules to a nucleic acid of Table 1 or Table 2.

18. An antibody which specifically binds to a protein encoded by a nucleic acid of Table 1 or Table 2 or a fragment thereof.

19. The antibody of claim 18, wherein said antibody is a monoclonal antibody.

20. The antibody of claim 18, wherein said antibody is a humanized antibody.

21. The antibody of claim 18, wherein said antibody is an antibody fragment.

22. A biochip comprising one or more nucleic acid segments selected from the group consisting of a nucleic acid of Table 1 or Table 2 or a fragment thereof, wherein said biochip comprises fewer than 1000 nucleic acid probes.

23. A nucleic acid having a sequence at least 95% homologous to a sequence of a nucleic acid of Table 1 or Table 2 or its complement.

24. A nucleic acid which hybridizes under high stringency to a nucleic acid of Table 1 or Table 2 or its complement.

25. A polypeptide encoded by the nucleic acid of claim 23 or 24.

26. A method of eliciting an immune response in an individual, said method comprising administering to said individual a composition comprising the polypeptide of claim 25 or a fragment thereof.

27. A method of eliciting an immune response in an individual, said method comprising administering to said individual a composition comprising a nucleic acid comprising a sequence of a nucleic acid of Table 1 or Table 2 or a fragment thereof.

28. A method of determining the prognosis of an individual with colorectal cancer comprising:
a) determining the expression of one or more genes selected from the group consisting of a nucleic acid of Table 1 or Table 2 or a fragment thereof in a first tissue type of a first individual; and
b) comparing said expression of said gene(s) from a second normal tissue type from said first individual or a second unaffected individual;
wherein a substantial difference in said expression indicates a poor prognosis.

29. A method of treating colorectal cancer comprising administering to an individual having colorectal cancer an antibody to a colorectal cancer modulator protein or fragment thereof conjugated to a therapeutic moiety.

30. The method of claim 29, wherein said therapeutic moiety is a cytotoxic agent.

31. The method of claim 29, wherein said therapeutic moiety is a radioisotope.
